(19) United States
(12) Patent Application Publication
(10) Pub. No.: US 2026/0356783 A1
(43) Pub. Date: Nov. 12, 2026

(54) SYNTHESIS OF ALPHA,BETA-UNSATURATED CARBONYLS FROM ALKENES VIA SULFONIUM INTERMEDIATES AND THEIR APPLICATION IN THE SYNTHESIS OF CONJUGATED DIENE PHEROMONES, KAIROMONES, AND RELATED COMPOUNDS

(71) Applicants: HUN-REN TERMÉSZETTUDOMÁNYI KUTATÓKÖZPONT, Budapest (HU); EÖTVÖS LORÁND TUDOMÁNYEGYETEM, Budapest (HU)

(72) Inventors: Péter ANGYAL, Nyíregyháza (HU); András Miklós KOTSCHY, Törökbálint (HU); Ádám DUDÁS, Budapest (HU); Tibor SOÓS, Budapest (HU); Szilárd VARGA, Szentgotthárd (HU)

(21) Appl. No.: 19/131,046

(22) PCT Filed: Nov. 17, 2023
(86) PCT No.: PCT/HU2023/050081

§ 371 Date: May 19, 2025

(30) Foreign Application Priority Data
Nov. 17, 2022 (HU) P2200454

(51) Int. Cl. C07C 45/56; C07C 51/377; C07C 67/297; C07C 253/30; C07D 209/44

(52) U.S. Cl. CPC C07C 45/567; C07C 51/377; C07C 67/297; C07C 253/30; C07D 209/44

(57) ABSTRACT

α,β-Unsaturated aldehydes are useful products as such in agrochemistry, but also potential intermediates for the preparation of important insect pheromones and kairomones. Until now, primarily the thermodynamically more stable (E)-α,β-unsaturated aldehydes have been identified as useful intermediates in the production of conjugated diene pheromone compounds, however, their practical and cost-effective synthesis on an industrial scale remains challenging. The problem to be solved by the present invention is to provide a method for selective oxidation of alkenes to α,β-unsaturated carbonyls which can be further transformed into pheromones and kairomones preferably without isolation and significant loss of the existing stereochemical purity.

### FIELD OF THE INVENTION

The present invention(s) relates to a method for the preparation and further transformation of (α,β-unsaturated carbonyls wherein said method comprises the following steps: i) an alkene is reacted to obtain an alkenyl sulfonium salt or synthetic equivalent; ii) the obtained sulfonium salt is oxidized with an amine-oxide or sulfoxide to form an α,β-unsaturated carbonyl; iii) optionally, the resulting α,β-unsaturated carbonyl is separated and purified or directly transformed into a conjugated diene pheromone or a kairomone/related compound.

### BACKGROUND OF THE INVENTION

α,β-unsaturated aldehydes are useful products as such in agrochemistry, but also potential intermediates for the preparation of important insect pheromones and kairomones. Until now, primarily the thermodynamically more stable (E)-α,β-unsaturated aldehydes have been identified as useful intermediates in the production of conjugated diene pheromone compounds, however, their practical and cost-effective synthesis on an industrial scale remains challenging. In addition, as control and preservation of olefin geometry require eluding the thermodynamic driving force, synthesis, and further transformation of (Z)-α,β-unsaturated aldehydes prove to be even more scarce.

As terminal alkenes are easily available and relatively cheap starting materials, their selective transformations are one of the fundamental reactions in organic chemistry which are used in many large-scale chemical processes and academic laboratories. Nonetheless, transforming olefins into α,β-unsaturated aldehydes on an industrial scale has been mostly limited to cross-metathesis reactions with α,β-unsaturated aldehydes (e.g. acrolein) (Ref i) or their masked equivalents (e.g. acrolein diethyl acetal) (Ref ii). These transformations furnish prolonged chain (one carbon atom longer) products with good selectivity altered by a properly chosen metathesis catalyst (e.g. ruthenium- and molybdenum-based catalysts) to achieve (E) or (Z) selectivity. Unfortunately, the more widespread use of these methodologies is hindered by the necessity of expensive catalyst systems and the use of difficult-to-handle short-chain α,β-unsaturated aldehydes.

A fundamentally different approach would be the oxidative transformation of terminal alkenes to α,β-unsaturated aldehydes, which process does not involve the incorporation of carbon atoms into the chain. However, only a few publications describe such oxidations. Of these limited cases, most produce the α,β-unsaturated aldehydes in low yield and poor selectivity over other oxidation products, and/or require activated allylic CH bonds (i.e. allylbenzenes) or directing groups (i.e. lactams) in the presence of a transition metal catalyst (e.g. palladium- and iron-based catalysts). (Ref iii) Importantly, there is no precedent for the regioselective transformation of unbiased alpha-olefins (non-activated, no directing group; e.g. fatty olefin derivatives) with such methodologies, and no attainable (Z)-selectivity among these procedures has been reported to date.

The recently discovered chemistry of different reactive thianthrenium salts derived from unbiased alkenes seems to create a new opportunity in olefin chemistry. (Ref iv) This activation principle relies on the synthesis of covalent sulfonium species, which highly reactive molecules might be converted into more valuable compounds with or without their isolation. As seminal works illuminated the possibility of producing and transforming these species, their narrow scope indicated that there is room for further improvements. (Ref v) The introduction of thianthrene into this chemistry by Ritter's group enabled the synthesis of alkenyl thianthrenium salts from abroad range of olefins in a stereo- and regioselective manner and these salts were further transformed via cross-coupling reactions. (Ref vi) As outlined in Chen's doctoral dissertation, (Ref vii) these alkenyl thianthrenium species were also found to engage in reactions consisting of direct nucleophilic replacements. These seminal experiences could be explained by three types of thianthrenium reactivity, more specifically: Type I: reaction as a vinyl cation equivalent giving a β-carbon substituted product (e.g. reaction with cyanide); Type II: reaction as a dication equivalent to afford cyclic or disubstituted products (e.g. reaction with primary amines). Type III: reaction as an allylic cation with the double bond shifted (e.g. reaction with carboxylic acids).

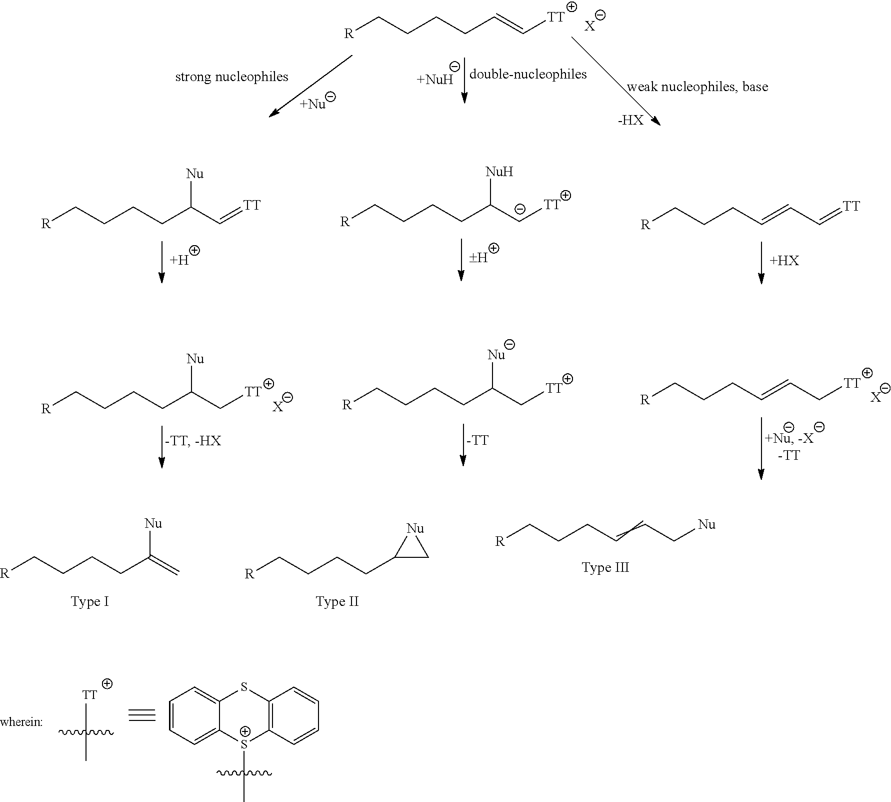

Then, Wickens demonstrated that dicationic and related metastable bis-thianthrenium adducts are readily generated employing electrochemistry and their reactivities can be directed to converge and provide similar reactivities as above, including the formation of vinyl nitriles (Type I), aziridines, diamines, dihalogenides (Type II) or allylic amines (Type III). (Ref viii) At the same time, Shu reported analogous allylic functionalizations via alkenyl thianthrenium salts to attain allylic amination, esterification, etherification, and arylation (Type III). (Ref ix)

Summarizing the prior knowledge regarding Type III reactivity to date, these non-typical transformations are all enabled by the oxidative activation of olefins via sulfonium species followed by a redox-neutral, preferably (Z)-selective substitution event. More specifically, these sulfonium species may consist of alkenyl sulfonium salts, bridged dicationic, and bis-adducts, which can be treated as synthetic equivalents in general terms. These intermediates are obtained from the reaction of olefins and activated sulfonium reagents prepared by either means of chemical activation (e.g. reacting thianthrene S-oxide with trifluoromethanesulfonic anhydride) or electrochemical synthesis (e.g. anodic oxidation of thianthrene). The main challenge of this chemistry lies in controlling the reactivity of these species in the presence of nucleophiles, thus redounding the desired downstream transformation (i.e. Type III) and suppressing other possible reactivities (e.g. Type I). To the best of our knowledge, there are no reports describing the successful reaction of the said alkenyl sulfonium salts or synthetic equivalents with nucleophilic oxidizing agents (i.e. sulfoxides and amine-oxides), in particular including the Type III reactivity to furnish α,β-unsaturated carbonyls.

The oxidation of alkyl halides to the corresponding carbonyl compounds using dimethyl sulfoxide as a nucleophilic oxidant is known as the Komblum oxidation. As disclosed by Kornblum and co-workers in 1957, activated primary benzyl bromides and α-bromo aromatic ketones were readily oxidized to the corresponding aldehydes and phenylglyoxals by dissolving the substrates in dimethyl sulfoxide. (Ref x) However, this procedure gave good yields only in the case of activated halides, less reactive ones (e.g. aliphatic alkyl halides) remained intact. Hence, several improvements and variants of the transformation have been developed since, (Ref xi) including the use of amine-oxides as alternative nucleophilic oxidants (called the Ganem oxidation). (Ref xii) As sulfoxides and amine-oxides are generally weak nucleophiles, the scope of (pseudo)halogenides (in reactivity order: tosylate>iodide>bromide>chloride) that can be used directly in this reaction proved to be limited and the drawbacks of these reactions remain as stated: the common need for activation (e.g. as tosylates), harsh conditions (i.e. elevated temperatures), poor chemoselectivity, and moderate yields. Regarding the use of alkyl sulfoniums in such transformation, there is one single report describing that dissolving (9-Anthrylmethyl)dimethylsulfonium hydrogensulfate in DMSO-d₆ resulted in the formation of 9-anthraldehyde. (Ref xiii) This example however is a specific case due to the activation of the benzylic position, and gives no clue about the reactivity of alkenyl sulfonium salts or synthetic equivalents derived from alkenes and the possibility to synthesize α,β-unsaturated carbonyls. To the best of our knowledge, there are no reports describing the use of sulfonium salts as substrates in the Kornblum reaction to obtain α,β-unsaturated carbonyls.

### The Problem to be Solved by the Invention

The problem to be solved by the present invention is to provide a method for selective oxidation of alkenes to α,β-unsaturated carbonyls, where the use of said methodology has the following features:
a) capability to transform unbiased (non-activated, no directing group) alkenes, especially aliphatic terminal alkenes (e.g. fatty olefin derivatives) into α,β-unsaturated aldehydes,
b) controllable and high E/Z selectivity (if relevant) preferring either the selective formation of the (E)-α,β-unsaturated aldehyde or the (Z)-α,β-unsaturated aldehyde product, as needed,
c) compatibility to further transform the product (either the (E) or the (Z)-α,β-unsaturated aldehyde) obtained, preferably without isolation and significant loss of the existing stereochemical purity. This includes generally known transformations, especially reactions with phosphonium ylides to form (E,Z) or (Z,Z) or (Z,E) or (E,E) conjugated diene pheromones, kairomones, and related compounds,
d) high chemoselectivity, especially tolerating the presence of functional groups such as an ester/carboxylic acid/alcohol/aldehyde/halogen/pseudohalogen group,
e) excellent selectivity over other oxidation products,
f) mild operational conditions
g) practical work-up conditions to efficient removal of by- and side-products

### The Discovery According to the Present Invention

During our experiments, we found surprisingly that the above demands can be achieved by merging the Type III electrophilic reactivity of alkenyl sulfonium salts or synthetic equivalents with the nucleophilic reactivity of reagents known to be used in the Kornblum reaction (i.e. amine-oxides and sulfoxides). More specifically, a novel methodology has been developed based on this concept, consisting of the following steps: i) an alkene was reacted to obtain an alkenyl sulfonium salt or synthetic equivalent; ii) the obtained alkenyl sulfonium salt or synthetic equivalent was oxidized with an amine oxide or sulfoxide to form an α,β-unsaturated carbonyl. We also found that careful choice of the nucleophilic reagent, optional base, and conditions (especially regarding temperature) was enabling us to achieve the expected features of the methodology outlined above.

### BRIEF DESCRIPTION OF THE INVENTION

1. Provided herein are methods for preparing α,β-unsaturated carbonyls according to Formula I:

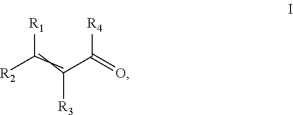

wherein
R1 is H; C1-C20 alkyl, preferably C1-C14 alkyl; C2-C20 alkenyl, preferably C2-C14 alkenyl; C2-C20 alkynyl, preferably C2-C14 alkynyl; —O—C1-C20 alkyl, preferably —O—C1-C14 alkyl; —O—C2-C20 alkenyl; —O—C2-C20 alkynyl; C3-C10 cycloalkyl; 4-10-membered heterocycloalkyl containing one or more heteroatoms independently selected from N, O and S; C6-C14 aryl, preferably phenyl; or 5-10-membered heteroaryl containing 1-3 heteroatoms independently selected from N, O and S;
   Wherein each alkyl, alkenyl, and alkynyl can be straight chained or branched, and each alkyl, alkenyl, alkynyl, cycloalkyl, heterocycloalkyl, heteroaryl and aryl can be substituted with 1-3 substituents independently selected from halogen, —CN, —OH, —OR25, —OTs, —OMs, —O—C(O)R21, ═O, —O—C₁-C₆ alkyl, —COOH, —C(O)—OR22, 1,3-dioxoisoindolin-2-yl, —NH—C(O)—C₁-C₆ alkyl, —N(C₁-C₆ alkyl)-C(O)—C₁-C₆ alkyl, —NHR23, —N(C₁-C₆ alkyl)R23, —C(O)—NH—C₁-C₆ alkyl, —C(O)—N(C₁-C₆ alkyl)₂, C3-C10 cycloalkyl, 4-10-membered heterocycloalkyl containing one or more groups independently selected from —NR23-, —O— or —S—, C6-C10 aryl, 5-10-membered heteroaryl containing 1-3 heteroatoms independently selected from N, O and S; or
R1 and R2 together with the carbon atom to which they are attached may form a saturated 5-6 membered cycloalkyl; or
R1 and R4 together with the carbon atoms to which they are attached may form a partially unsaturated 5-15-membered, preferably 5-8-membered cycloalkyl; or R1 and R4 may form a chain having 2-5 chain atoms and comprises CH2 units and/or 1-2 heteroatoms selected from N, O and S;
R2 is H, or together with R1, R2 may form cycloalkyl as described above;
R3 is H, C1-C4 alkyl, C6-C10 aryl, 4-methyltetrahydro-2H-pyran-2-yl;
with the proviso that R1 is other than H when all R2-R4 are H
R4 is H, C1-C4 alkyl, 4-methyltetrahydro-2H-pyran-2-yl; or together with R1, R4 may form cycloalkyl or chain as described above;
R21 is H; C1-C4 alkyl; or C6-C10 aryl, preferably phenyl;
R22 is C1-C4 alkyl, C2-C4 alkenyl or C2-C₄ alkynyl;
R23 is N-protecting group;
R25 is O-protecting group;
wherein the methods include the following step:
(i) reacting an alkenyl sulfonium salt of Formula III-1

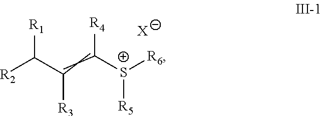

or a synthetic equivalent product thereof including
an allyl-sulfonium of Formula III-2

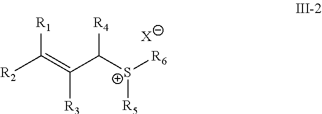

or a bis-adduct of Formula III-3

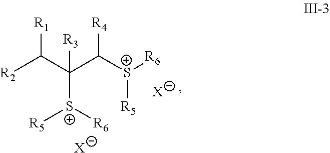

or a dicationic adduct of Formula III-4

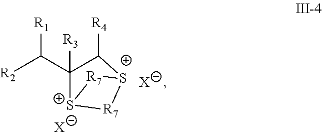

or a mixture thereof;
wherein R1-R4 has the meaning as described above,
R5 and R6 are selected from C1-C6 alkyl, C5-C7 cycloalkyl, C6-C10 aryl, wherein each of alkyl, cycloalkyl and aryl can be substituted with one or more substituents independently selected from halogen, C1-C2 alkyl, or a —(CH2)ₙ-heteroatom-(CH2)ₘ chain where n and m is independently 0 or 1 and the heteroatom is preferably selected from N, O and S, more preferably S or oxidized heteroatom (e.g. SO, SO2, more preferably SO2); preferably R5 and R6 are C6-C10 aryl, more preferably both aryl has at least one substituent in ortho position and said substituents of the aryls (R5 and R6) may form a direct bond between the aryls or a chain comprising CH2 units and/or a heteroatom (preferably selected from N, O and S, more preferably S) or oxidized heteroatom (e.g. SO, SO2, more preferably SO2),
more preferably R5 and R6 are both an optionally substituted phenyl
even more preferably the SR5R6 moiety is

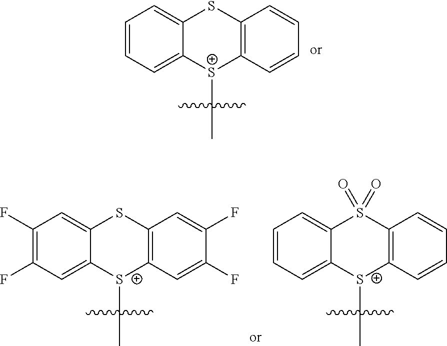 [SMILES: *[S+]1c2ccccc2Sc2ccccc21 ; *[S+]1c2cc(F)c(F)cc2Sc2cc(F)c(F)cc21 ; *[S+]1c2ccccc2S(=O)(=O)c2ccccc21]

X— is a suitable anion, preferably TfO⁻, MsO⁻, TsO⁻, TFA⁻, BF₄⁻, PF₆⁻, ClO₄⁻,
both R7 is ortho-phenylene which is optionally substituted as described above at R5 and R6;
with a nucleophilic oxidizing agent, preferably an amine-oxide or sulfoxide according to Formula IIIa or

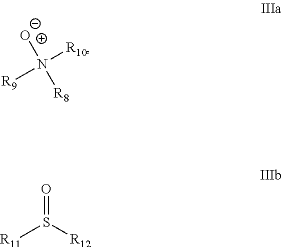

wherein R8, R9, and R10 are independently selected from C₁-C₄ alkyl, phenyl, or
R8 and R9 together with N atom to which they are attached, or R8-R10 together with N atom to which they are attached, may form a saturated, partially unsaturated or aromatic heterocyclic ring having 5-7 ring forming atoms, and comprising a N atom and may further comprise a heteroatom selected from N, O and S,
preferably, Formula IIIa is 4-Methylmorpholine N-oxide

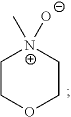

pyridine N-oxide or substituted pyridine N-oxide, for example 2,6-dimethylpyridine-N-oxide or 2-methylpyridine N-oxide; trimethylamine N-oxide, or N,N-diisopropylethylamine N-oxide;
R11 and R12 are independently selected form C₁-C₄ alkyl, or phenyl, preferably Formula IIIb is DMSO
optionally in the presence of an organic or inorganic base,
under conditions sufficient to form an unsaturated carbonyl product according to Formula I.

2. Method according to point 1, further comprising the steps of
(i) forming a reaction mixture comprising:
   an olefin according to Formula II:

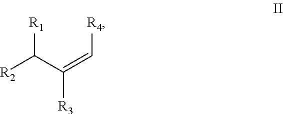

wherein R1-R4 have the meaning as described in point 1,
   a sulfoxide or sulfide according to Formula IIa or IIb:

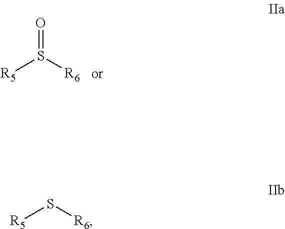

wherein R5 and R6 have the meaning as described in point 1, more preferably the compound of formula IIa is

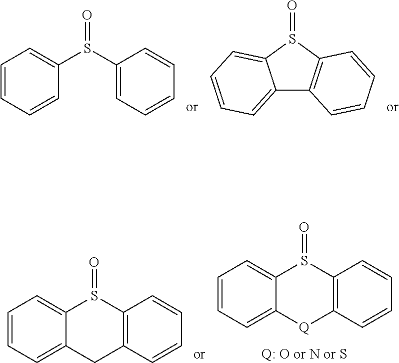

and even more preferably an optionally substituted

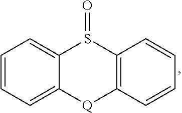

wherein Q is S (i.e. formula IIa is an optionally substituted TTO), and the optional substitution is a tetrafluoro substitution (i.e. formula IIa is

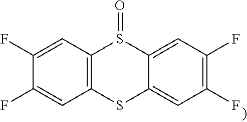 [SMILES: O=S1c2cc(F)c(F)cc2Sc2cc(F)c(F)cc21]

and the compound of formula IIb is

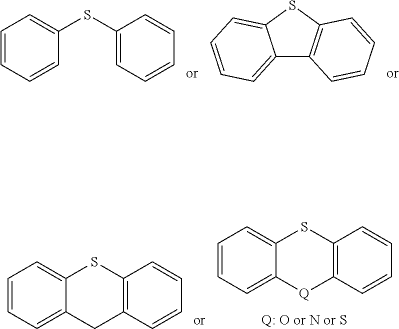

and even more preferably an optionally substituted

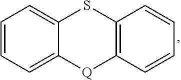

wherein Q is S (i.e. formula IIb is an optionally substituted TT), and the optional substitution is a tetrafluoro substitution (i.e. formula IIb is

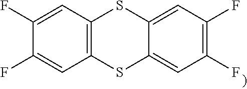 [SMILES: Fc1cc2c(cc1F)Sc1cc(F)c(F)cc1S2]

an activating agent or oxidizing agent, preferably carboxylic acid anhydride in the presence of an acid or 1-Chloromethyl-4-fluoro-1,4-diazoniabicyclo[2.2.2]octane bis(tetrafluoroborate, trifluoromethane sulfonic anhydride, or trifluoromethane sulfonic acid or methane sulfonic acid, optionally with trifluoroacetic anhydride or acetic anhydride, sodium tetrafluoroborate, tetra-n-butylammonium hexafluorophosphate, alkyl or aryl sulfonic acids, or combinations thereof, which might be replaced with an activating or oxidizing treatment (e.g. electrochemical oxidation), said agents and/or treatment provides X⁻ which is preferably TfO⁻, MsO⁻, TsO⁻, TFA⁻, BF₄⁻, PF₆⁻, ClO₄⁻,
(ii) maintaining the reaction mixture under conditions sufficient to form an alkenyl sulfonium salt of Formula III-1,

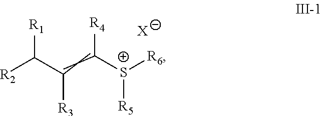

or a synthetic equivalent product including an allyl-sulfonium of Formula III-2

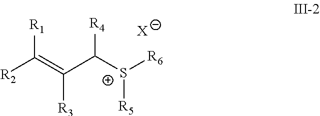

or a bis-adduct of Formula III-3

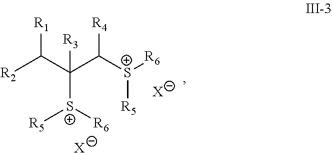

or a dicationic adduct of Formula III-4, if Formula IIa or IIb represent an optionally substituted TTO or TT

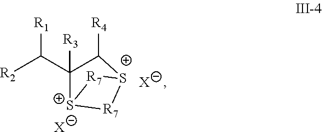

or a mixture thereof:
wherein R7 and X— have the meaning as described above.

3. Method according to point 1 or 2, wherein
R1 is H; C1-C14 alkyl; C2-C14 alkenyl; C2-C14 alkynyl; —O—C1-C14 alkyl; —O—C2-C14 alkenyl; —O—C2-C14 alkynyl; C3-C10 cycloalkyl; 4-10-membered heterocycloalkyl containing one or more heteroatoms independently selected from O and S; C6-C10 aryl, preferably phenyl; or 5-10-membered heteroaryl containing 1-3 heteroatoms independently selected from N, O and S;
   wherein each alkyl and alkenyl can be straight chain or branched, and each alkyl, alkenyl, alkynyl and aryl can be optionally substituted with 1-3 substituents independently selected from halogen, —CN, —OH, —OTs, —OMs, —O—C(O)R21, ═O, —COOH, —C(O)—OR22, 1,3-dioxoisoindolin-2-yl, —NH—C(O)—C1-C6 alkyl, —N(C1-C6 alkyl)-C(O)—C1-C6 alkyl, —NHR23, —N(C1-C6 alkyl)R23, —C(O)—NH—C1-C6 alkyl, —C(O)—N(C1-C6 alkyl)₂, C3-C7 cycloalkyl, 5-7-membered heterocycloalkyl containing one or more groups independently selected from —NR23-, —O— or —S—, C6-C10 aryl, 5-7-membered heteroaryl containing 1-3 heteroatoms independently selected from N, O and S, or —O—C1-C6 alkyl; or
R1 and R2 together with the carbon atom to which they are attached may form a saturated or partially unsaturated 5-6 membered cycloalkyl; or
R1 and R4 together with the carbon atoms to which they are attached may form a partially unsaturated 5-8-membered cycloalkyl; or R1 and R4 may form a chain having 2-3 chain atoms and comprises CH2 units and/or 1-2 heteroatoms selected from N, O and S;
R2 is H, or together with R1, R2 may form cycloalkyl as described above;
R3 is H, C1-C4 alkyl, phenyl, 4-methyltetrahydro-2H-pyran-2-yl;
with the proviso that RI is other than H when R2 and R3 are both H
R4 is H, C1-C4 alkyl, 4-methyltetrahydro-2H-pyran-2-yl; or together with R1, R4 may form cycloalkyl or chain as described above;
R21 is H; C1-C4 alkyl; or phenyl;
R22 is C1-C4 alkyl, C2-C4 alkenyl or C2-C4 alkynyl
R23 is N-protecting group.

4. Methods according to any of point 1-3, wherein R5 and R6 are C6-C10 aryl, which can be substituted with one or more substituents independently selected from halogen, C1-C2 alkyl, or a —(CH2)ₙ-heteroatom-(CH2)ₘH chain where n and m is independently 0 or 1 and n+m is 0 or 1, and the heteroatom is preferably selected from N, O and S, more preferably S or oxidized heteroatom (e.g. SO, SO2, more preferably SO2); preferably R5 and R6 are both phenyl, more preferably having at least one substituent in ortho position and said substituents of the phenyls (R5 and R6) may form a direct bond between the phenyls or a chain comprising CH2 units and/or a heteroatom (preferably selected from N, O and S, more preferably S) or oxidized heteroatom (e.g. SO, SO2, more preferably SO2), more preferably the compound of formula IIa is

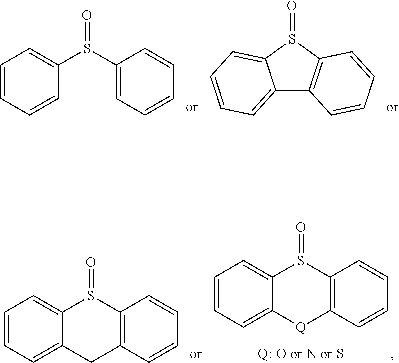

and even more preferably

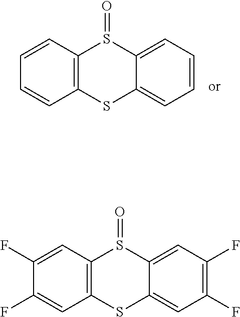 [SMILES: O=S1c2ccccc2Sc2ccccc21 ; O=S1c2cc(F)c(F)cc2Sc2cc(F)c(F)cc21]

and the compound of formula IIb is

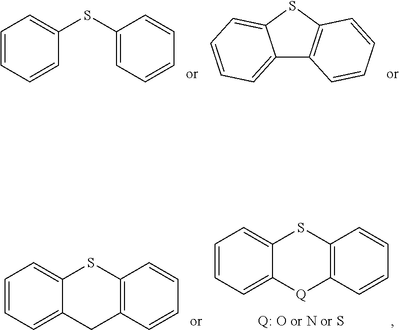

and even more preferably

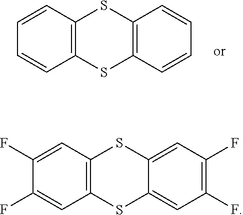 [SMILES: c1ccc2c(c1)Sc1ccccc1S2 ; "or" ; Fc1cc2c(cc1F)Sc1cc(F)c(F)cc1S2]

5. Methods according to any of points 1 to 4, wherein the activating agent or oxidizing agent is selected from 1-Chloromethyl-4-fluoro-1,4-diazoniabicyclo[2.2.2]octane bis(tetrafluoroborate, trifluoromethane sulfonic anhydride, trifluoromethane sulfonic acid or methane sulfonic acid optionally with trifluoroacetic anhydride or acetic anhydride, sodium tetrafluoroborate, tetra-n-butylammonium hexafluorophosphate or combinations thereof.

6. Methods according to any of points 1 to 5, wherein the nucleophilic oxidizing agent is selected from 4-Methylmorpholine N-oxide, pyridine N-oxide, 2-methylpyridine N-oxide, 2,6-dimethyl-pyridine-N-oxide, trimethylamine N-oxide, N,N-diisopropylethylamine N-oxide or DMSO, preferably 4-Methylmorpholine N-oxide or DMSO.

7. Methods according to any of points 1 to 6, wherein R1 is H; C1-C14 alkyl; C2-C6 alkenyl; C2-C6 alkynyl; —O—C1-C6 alkyl; or phenyl; wherein each alkyl, alkenyl and alkynyl can be straight chain or branched, and each alkyl, alkenyl, alkynyl and aryl can be substituted with 1-3 substituents independently selected from halogen, —CN, —OH, —OTs, —OMs, —O—C(O)R21, ═O, —O—C1-C4 alkyl, —COOH, —C(O)—OR22, 1,3-dioxoisoindolin-2-yl or phenyl; or
R1 and R2 together with the carbon atom to which they are attached may form a saturated or partially unsaturated 5-6 membered cycloalkyl; or
R1 and R4 together with the carbon atoms to which they are attached may form a partially unsaturated 5-8 membered cycloalkyl;
R2 is H, or together with R1, R2 may form cycloalkyl as described above;
R3 is H, C1-C4 alkyl, phenyl, 4-methyltetrahydro-2H-pyran-2-yl;
with the proviso that R1 is not H when R2 and R3 are both H
R4 is H, C1-C4 alkyl, 4-methyltetrahydro-2H-pyran-2-yl; or together with R1, R4 may form cycloalkyl as described above;
R21 is H; C1-C4 alkyl; or phenyl;
R22 is C1-C4 alkyl, or C2-C4 alkynyl.

8. Methods according to any of points 1 to 7, wherein R2 and R3 are H; or alternatively R3 and R4 are H; or alternatively R2, R3 and R4 are H.

9. Methods according to any of points 1 to 8, wherein R1 is C1-C14 alkyl; which is optionally substituted, preferably with 1-3 substituents independently selected from halogen, —CN, —OH, —OTs, —OMs, —O—C(O)R21, ═O, —O—C1-C4 alkyl, —COOH, —C(O)—OR22, 1,3-dioxoisoindolin-2-yl or phenyl.

10. Methods according to any of points 1 to 8, wherein the compound of Formula IIa or Formula IIb is

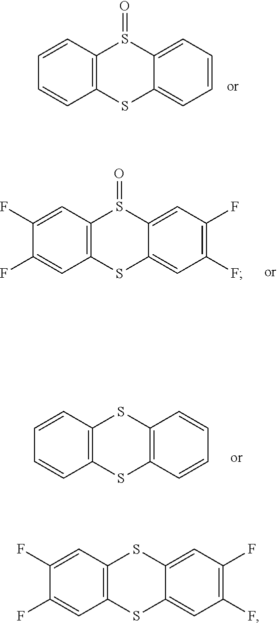 [SMILES: O=S1c2ccccc2Sc2ccccc21 ; O=S1c2cc(F)c(F)cc2Sc2cc(F)c(F)cc21 ; "or" ; c1ccc2c(c1)Sc1ccccc1S2 ; "or" ; Fc1cc2c(cc1F)Sc1cc(F)c(F)cc1S2]

respectively.

11. Methods according to any of points 1 to 10, wherein the α,β-unsaturated carbonyls according to Formula I is selected from:
(Z)-pent-2-enal; (Z)-octadec-2-enal; 2-phenylacrylaldehyde; 2-cyclohexylideneacetaldehyde; (Z)-3-(3-phenylpropoxy)acrylaldehyde; (Z)-7-oxohept-5-en-1-yl benzoate; (Z)-6-oxohex-4-enenitrile; (Z)-6-(1,3-dioxoisoindolin-2-yl)hex-2-enal; (Z)-11-oxoundec-9-enoic acid; (Z)-10-hydroxydec-2-enal; (Z)-dec-2-enedial; (Z)-octa-2,7-dienal; prop-2-yn-1-yl (Z)-11-oxoundec-9-enoate; cinnamaldehyde; (Z)-6-chlorohex-2-enal; (Z)-7-bromohept-2-enal; (Z)-10-oxodec-8-en-1-yl 4-methylbenzenesulfonate; cyclopent-2-en-1-one; cyclohex-2-en-1-one; cyclohept-2-en-1-one; cyclooct-2-en-1-one; (E)-hex-3-en-2-one; (E)-hex-4-en-3-one; 2-methyl-1-((2R,4S)-4-methyltetrahydro-2H-pyran-2-yl)prop-2-en-1-one; (E)-2-methyl-3-((2R,4S)-4-methyltetrahydro-2H-pyran-2-yl) acrylaldehyde; methyl (E)-10-oxodec-8-enoate; methyl (Z)-11-oxoundec-9-enoate; (E)-9-oxonon-7-en-1-yl acetate; (Z)-11-oxoundec-9-en-1-yl acetate; (Z)-oct-2-enal; (Z)-11-hydroxyundec-2-enal.

12. Methods according to any of points 1 to 11, wherein the reaction is performed in non-nucleophilic or poorly-nucleophilic solvents or solvent mixtures, preferably selected from ethers (e.g., glymes, tetrahydrofuran, methyltetrahydrofuran, diethyl ether, methyl tert-butyl ether, diisopropyl ether, dibutyl ether or 1,4-dioxane), aliphatic hydrocarbons (e.g. hexane, heptane, petroleum ether, octane, or cyclohexane), aromatic hydrocarbons (e.g. benzene, toluene, xylene or mesitylene), halocarbons (e.g. dichloromethane, chloroform, dichloroethane), carbonyls (e.g. acetone) and additional polar aprotic solvents (e.g. acetonitrile, dimethylformamide, dimethyl sulfoxide), or a mixture thereof.

13. Methods according to any of points 1 to 12, wherein the reaction is performed at a temperature of −40 °C to 80 °C, preferably at a temperature within the range of −20 to 50 °C

14. Methods according to any of points 1 to 13, wherein the reaction is performed in the presence of an organic or inorganic base, preferably selected from N,N-diisopropylethylamine, sodium bicarbonate, sodium carbonate, potassium carbonate.

15. A further object of this invention is to provide compatible methods to transform the above unsaturated carbonyl products according to Formula I, preferably without isolation and significant loss of the existing stereochemical purity, into conjugated diene pheromones, kairomones, and related compounds according to Formula V.

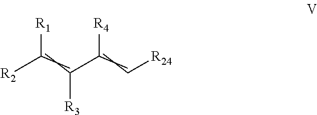

wherein R24 is H; C1-C20 alkyl, preferably C1-C14 alkyl; C2-C20 alkenyl, preferably C2-C14 alkenyl; C2-C20 alkynyl, preferably C2-C14 alkynyl; —O—C1-C20 alkyl, preferably —O—C1-C14 alkyl; or C6-C14 aryl, preferably phenyl; 5-10-membered heteroaryl containing 1-3 heteroatoms independently selected from N, O and S; wherein each alkyl and alkenyl can be straight chained or branched, and each alkyl, alkenyl and aryl can be substituted with 1-3 substituents independently selected from halogen, —CN, —OH, —OTs, OMs, —O—C(O)R21, ═O, —COOH, —C(O)—OR22, 1,3-dioxoisoindolin-2-yl, —NH—CO—C1-C6 alkyl, —N(C1-C6 alkyl)-CO—C1-C6 alkyl, —NHR23, —N(C1-C6 alkyl)R23, —CO—NH—C1-C6 alkyl, —CO—N(C1-C6 alkyl)₂, C6-C10 aryl, 5-10-membered heteroaryl containing 1-3 heteroatoms independently selected from N, O and S, or —O—C1-C6 alkyl,
wherein the methods include:
(i) forming a reaction mixture comprising:
an unsaturated aldehyde according to Formula I,
a phosphonium ylide according to Formula IVa:

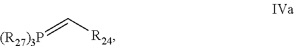

wherein R24 has the meaning as described above, and
R27 is aryl, preferably phenyl;
(ii) maintaining the reaction mixture under conditions sufficient to form a conjugated diene product according to Formula V.

The preparation of the phosphonium ylide according to Formula IVa is well known in the art, preferably generated in situ from an appropriate phosphonium salt in the presence of a base.

### DETAILED DESCRIPTION OF THE INVENTION

### Definitions

The product of the method according to the invention is α,β-unsaturated carbonyl, and in a preferred embodiment an α,β-unsaturated aldehyde.

The starting materials of the method are sulfonium salts; alkenyl sulfonium salts or synthetic equivalents thereof; in a preferred embodiment thianthrenium salts; alkenyl thianthrenium salts, (bridged) dicationic thianthrenium adducts, bi-thianthrenium adducts, covalent sulfonium species.

The following are used to prepare the above starting materials according to a method disclosed herein: alkenes, olefins, biased or unbiased alkenes, unactivated alkenes, (aliphatic) terminal alkenes, fatty olefin derivatives, alpha-olefins, disubstituted olefins, trisubstituted olefins.

As used herein, the term “halo” or “halogen” means fluorine, chlorine, bromine or iodine, preferably fluorine, chlorine or bromine, even more preferably fluorine or chlorine.

As used herein, the term “alkyl” alone or in combinations means a straight or branched-chain saturated hydrocarbon group containing from 1 to 20, preferably 1 to 14 carbon atom(s) (i.e. “C1-C20” or “C1-C14” alkyl groups), such as methyl, ethyl, propyl, isopropyl, butyl, sec-butyl, tert-butyl and pentyl, etc. In an embodiment, this phrase can relate to alkyl groups containing from 1 to 6, or 1 to 4, or 1 to 2 carbon atom(s) (i.e. “C1-C6” or “C1-C4” or “C1-C2” alkyl groups).

As used herein, the term “alkenyl” alone or in combinations means a straight or branched-chain hydrocarbon group containing from 2 to 20 carbon atom(s) or 2 to 14 carbon atom(s), one or more carbon-carbon double bonds, and no triple bonds (i.e. “C2-C20” or “C2-C14” alkenyl groups). In an embodiment, this phrase can relate to alkenyl groups containing from 2 to 6, or 2 to 4 (i.e. “C2-6” or “C2-4” alkenyl groups). Examples are ethenyl, 1-propenyl, 2-propenyl, 1-butenyl, 2-butenyl, butadienyl, pentenyl, pentadienyl, hexenyl, and the like. In an embodiment terminal double bond is preferred.

As used herein, the term “alkynyl” alone or in combinations means a straight or branched-chain hydrocarbon group containing from 2 to 20 carbon atom(s) or 2 to 14 carbon atom(s), one or more carbon-carbon triple bonds, and optionally one or more double bonds (i.e. “C2-C20” or “C2-C14” alkynyl groups). In an embodiment, this phrase can relate to alkynyl groups containing from 2 to 6, or 2 to 4 (i.e. “C2-C6” or “C2-C4” alkynyl groups). Examples are ethynyl, 1-propynyl, 2-propynyl, 1-butynyl, 2-butynyl, pentynyl, hexynyl, and the like. In an embodiment terminal triple bond is preferred.

As used herein the term “cycloalkyl”, alone or in combinations means a saturated or partially unsaturated cyclic hydrocarbon group having from 3 to 10 ring carbon atoms (“C3-C10 cycloalkyl”) and zero heteroatoms in the non-aromatic ring system. In special cases, this phrase can relate to cycloalkyl groups containing from 3 to 8, or 3 to 6 (i.e. “C3-8” or “C3-6” cycloalkyl groups). Non-limiting examples are cyclopropyl, cyclopropenyl, cyclobutyl, cyclobutenyl, cyclopentyl, cyclopentenyl, cyclohexyl, cyclohexenyl, cycloheptyl, cycloheptenyl, cyclooctyl, cyclooctenyl, bicyclo[2.2.1]heptanyl, bicyclo[2.2.2]octanyl, cyclononyl, cyclononenyl, cyclodecyl, cyclodecenyl, octahydro-1H-indenyl, decahydronaphthalenyl, and spiro[4.5]decanyl. The cycloalkyl group is either monocyclic or contains a fused, bridged, or spiro ring system such as a bicyclic system. “cycloalkyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Preferred cycloalkyl groups are cyclopropyl, cyclobutyl, cyclopentyl, cyclohexyl.

As used herein the term “heterocyclyl” or “heterocycloalkyl” alone or in combinations means a saturated or partially unsaturated non-aromatic ring system having 3 to 9 ring carbon atoms and 1 to 4 ring heteroatoms, wherein each heteroatom is independently selected from nitrogen, oxygen, sulfur, boron, phosphorus, and silicon (“4-10 membered heterocyclyl”). In special cases, this phrase can relate to heterocyclyl groups having 4-8 ring forming atoms (“4-8 membered heterocyclyl”) or 4-6 ring forming atoms (“4-6 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”). Non-limiting examples are azirdinyl, oxiranyl, thiorenyl, azetidinyl, oxetanyl, thietanyl, tetrahydrofuranyl, dihydrofuranyl, tetrahydrothiophenyl, dihydrothiophenyl, pyrrolidinyl, dihydropyrrolyl, pyrrolyl-2,5-dione, dioxolanyl, oxasulfuranyl, disulfuranyl, oxazolidin-2-one, triazolinyl, oxadiazolinyl, thiadiazolinyl, piperidinyl, tetrahydropyranyl, dihydropyridinyl, thianyl, piperazinyl, morpholinyl, dithianyl, dioxanyl, triazinanyl, azepanyl, oxepanyl, thiepanyl, azocanyl, oxecanyl, thiocanyl, indolinyl, isoindolinyl, dihydrobenzofuranyl, dihydrobenzothienyl, benzoxazolinonyl, tetrahydroquinolinyl, tetrahydroisoquinolinyl, and the like. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is either on the carbocyclic or heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system.

As used herein the term “aryl”, alone or in combinations means an aromatic monocyclic or polycyclic ring system comprising 6 to 10 carbon atoms. Nonlimiting examples of suitable aryl groups include phenyl, and naphthyl, where phenyl is a preferred embodiment.

The term “heteroaryl” means a group derived from a monocyclic or bicyclic aromatic ring system (condensed double ring systems) with 1 to 3 ring forming heteroatom(s) selected from the group of N, O and S and 3 to 9 ring forming carbon atoms (“5-10 membered heteroaryl”). In special cases, this phrase can relate to heteroaryl groups having 5-8 ring forming atoms (“5-8 membered heteroaryl”) or 5-6 ring forming atoms (“5-6 membered heteroaryl”). “Heteroaryl” includes ring systems wherein the heteroaryl ring, as defined above, is fused with one or more carbocyclic or heterocyclic groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Non-limiting examples are pyrrolyl, furanyl, thiophenyl, imidazolyl, pyrazolyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl, triazinyl tetrazinyl, azepinyl, oxepinyl, thiepinyl, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, purinyl, naphthyridinyl, pteridinyl, quinolinyl, isoquinolinyl, cinnolinyl, quinoxalinyl, phthalazinyl, and quinazolinyl. In heteroaryl groups that contain one or more heteroatoms, the point of attachment can be a carbon or heteroatom, as valency permits.

O- and N-protecting groups are well known in the art and include those described in detail in Protecting Groups in Organic Synthesis, T. W. Greene and P. G. M. Wuts, 3rd edition, John Wiley & Sons, 1999. Preferred examples are acetyl, benzoyl, benzyl, trityl, ethers, Boc, Fmoc, carbobenzyloxy carbamate and tosyl group. If any of R1-R4 of compound of formula I-III contains or represents a heterocycle or a heteroaromatic ring, which comprises nitrogen atom, said nitrogen atom is preferably protected.

Ts is tosyl group. Ms is mesyl group. Tf is triflate group.

### Methods

During our research, we investigated the feasibility of a novel methodology, more specifically, the transformation of alkenyl sulfonium salts or synthetic equivalents derived from alkenes into α,β-unsaturated carbonyls. The enabling concept behind such methodology would be merging the Type III electrophilic reactivity of alkenyl sulfonium salts or synthetic equivalents with the nucleophilic reactivity of reagents known to be used in the Kornblum reaction (i.e. amine-oxides and sulfoxides). As no precedent was known that indicated the plausibility of this concept, we decided to study a model reaction according to the following reaction scheme:

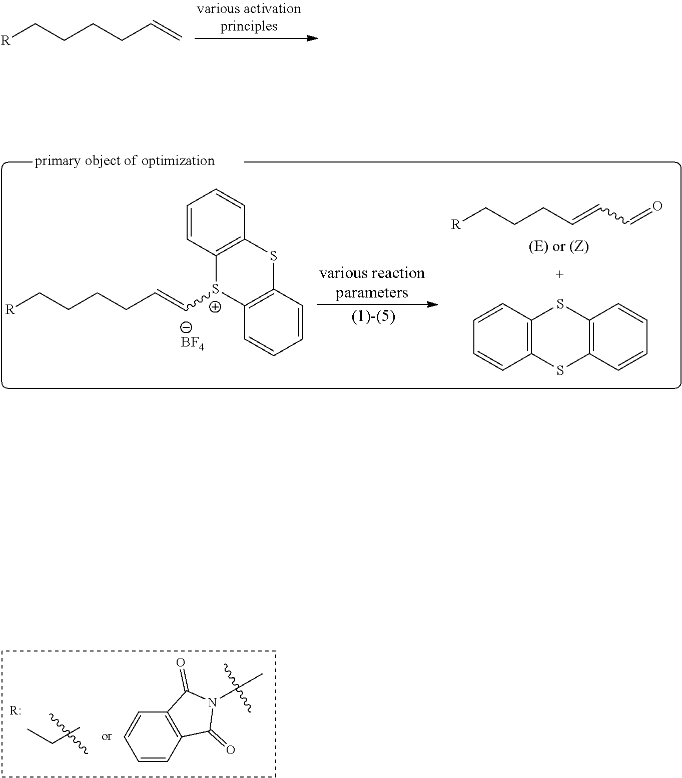

5-(6-(1,3-dioxoisoindolin-2-yl)hex-1-en-1-yl)-5H-thianthren-5-ium tetrafluoroborate and 5-(oct-1-en-1-yl)-5H-thianthren-5-ium tetrafluoroborate (both synthesized from the corresponding alkenes) were chosen as model compounds and several reaction parameters were probed including the use of different nucleophilic oxidizing agents (1), bases (2), conditions (3), execution and workup alternatives (4) and robustness (5) to achieve the desired transformation.

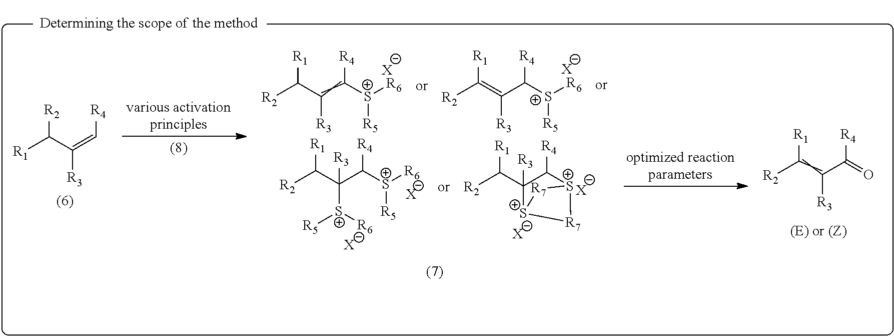

Then, according to the above scheme, the scope of the method was examined, including the range of suitable substrates (6) (e.g. type of alkenes, substituents, and functional groups present), acceptable sulfonium species (7), compatible activation processes (8) (e.g., activating/oxidizing agents or treatments to synthesize these species, one-pot modifications).

Finally, further transformations of the α,β-unsaturated carbonyl products prepared according to the present innovation into conjugated diene pheromones, kairomones, and related compounds have been accomplished and demonstrated by non-limiting examples (9) according to the following reaction scheme:

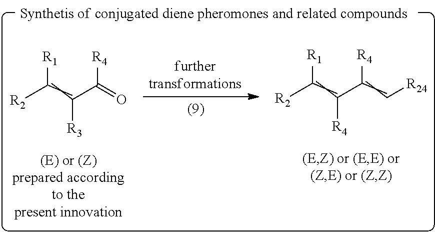

During our studies, a significant amount of experience has been gained that underpins our discovery and several problems have been solved to achieve the expected features of this novel methodology;

(1) Using 4-methylmorpholine N-oxide as a nucleophilic oxidizing agent is found to be a practical, industrially acceptable choice with fine-tuned reactivity. More specifically, excellent selectivity over other oxidation products and N-alkylated side products is observed. Aromatic N-oxides ((i.e. 2-methylpyridine N-oxide), sterically less hindered N-oxides (i.e. trimethylamine N-oxide) or sterically strongly crowded reagents (i.e. N,N-diisopropylethylamine N-oxide) give worse results, however, some reactivity is observed in several cases. As in the classical Kornblum reaction, this novel transformation is not restricted to the use of amine-oxides as nucleophilic oxidizing agents; the use of sulfoxides (i.e. dimethyl sulfoxide) as an oxygen source also results in product formation. Here we state that the optimal reagent is dictated by the substrate in question: i.e. 4-methylmorpholine N-oxide generally performs better in the case of alpha-, 1,1-disubstituted-, trisubstituted-olefins, and dimethyl sulfoxide proves to be higher-yielding for 1,2-disubstituted-olefins. In some embodiments, excess of the reagent (e.g. use of 4.0 equivalents of 4-methylmorpholine N-oxide) may result in higher yields and diminished side product formation. In some specific cases, using the reagent as a solvent secures beneficial results (i.e. use of dimethyl sulfoxide as a reagent and solvent).

(2) A wide range of organic (e.g. N,N-diisopropylethylamine) and inorganic bases (e.g. sodium bicarbonate, potassium carbonate) might be appropriate to promote the desired reaction. In some embodiments, different bases might have different advantages: i) inorganic bases are easily removed by an aqueous work-up aside from the desired α,β-unsaturated carbonyl products (prone to isomerization); ii) organic bases might enable homogenous reaction mixtures and wider temperature ranges due to better solubility. However, some basic limitations have been identified: the presence of nucleophilic external bases (i.e. 1,8-Diazabicyclo(5.4.0)undec-7-ene) or ones derived from amine-oxides as byproducts (i.e. trimethylamine derived from trimethylamine N-oxide) result in N-alkylated side product formation and diminished yields. Here we mention that the use of an external base might be optional, as autocatalysis may also deliver positive results (i.e. autocatalysis by 4-methylmorpholine derived from 4-methylmorpholine N-oxide).

(3a) The methods operate well in the presence of non-nucleophilic or poorly-nucleophilic solvents or solvent mixtures. The term “non-nucleophilic solvent’ is one known to those skilled in the art; in the present case, it refers to a solvent that is not a free primary alcohol or amine or an organic acid. “Poorly nucleophilic solvents' include those which may contain nucleophilic moieties, but which moieties are sterically or otherwise hindered. Non-nucleophilic or poorly nucleophilic solvents can include ethers (e.g., glymes, tetrahydrofuran, methyltetrahydrofuran, diethyl ether, methyl tert-butyl ether, diisopropyl ether, dibutyl ether or 1,4-dioxane), aliphatic hydrocarbons (e.g. hexane, heptane, petroleum ether, octane, or cyclohexane), aromatic hydrocarbons (e.g. benzene, toluene, xylene or mesitylene), halocarbons (e.g. dichloromethane, chloroform, dichloroethane), carbonyls (e.g. acetone) and additional polar aprotic solvents (e.g. acetonitrile, dimethylformamide, dimethyl sulfoxide), or a mixture thereof. Deuterated solvent can be also applied, like tetrahydrofuran-d₈. If only reactivity is considered, several solvents might be good alternatives, however, some might have practical advantages: e.g. acetonitrile has the advantage of not being miscible with aliphatic hydrocarbons (e.g. hexanes). This allows washing the reaction mixture to remove the by-product thianthrene, if necessary.

(3b) The reaction temperature can vary within a wide range of values and will in general be in the range of −40 °C to 80 °C The temperature and reaction time chosen will depend on the desired E/Z selectivity and the reactivity of the substrate, and can be adjusted accordingly without difficulty. Preferably, the reaction is conducted at a temperature within the range of −20 to 50 °C Importantly, temperature and reaction time substantially affect the geometric outcome of the reaction (i.e. the E:Z ratio of the α,β-unsaturated carbonyl product) if relevant, and reversal of this selectivity is generally achievable within this range. More specifically, selective formation of the (Z)-α,β-unsaturated aldehyde is preferably conducted at temperatures in the range of −20 to 0 °C and selective formation of the (E)-α,β-unsaturated aldehyde is preferably conducted at temperatures in the range of 25 to 50 °C

In general, elevated temperatures and prolonged reaction times promote the selective formation of the thermodynamically favoured E isomer and lower temperatures combined with minimal reaction times enable the selective formation of the elusive Z isomer.

(3c) The pressure applied in the reactions is atmospheric in general. However, elevated pressure (e.g. 2 to 10 atm) can be useful, especially if one of the components is a gas or a highly volatile compound (e.g. crotonaldehyde).

(4a) The order of the addition of the reactants is also interchangeable. Premixing either two of the components (sulfonium salt, oxidizing/activating reagent, and base) and dropwise addition of the third reactant is possible. Here we mention, that premixing the base and oxidizing reagent and slow addition of the sulfonium salt might be beneficial as more precise temperature control can be achieved.

(4b) Aqueous work-up of the reaction is not necessary; however, it is an effective and practical way to remove different side- and by-product components of the reaction mixture. This can be explained by the large difference in polarity between the desired product in general and these different components including inorganic bases, N-alkylated side products, and the excess amine-oxide reagents. In general, the obtained residue after work-up may be distilled, partitioned between two phases (e.g. hexanes/acetonitrile), chromatographed or steam distilled for further purification (carried out in line with the general knowledge of a skilled person), if needed.

(4c) Polar enough substrates (which are not well soluble in aliphatic hydrocarbons) can be isolated with good yield and purity utilizing only extractions. This modification results in minimal or no E/Z isomerization and comprises the following steps: i) conducting the reaction in a solvent not miscible with aliphatic hydrocarbons, preferably acetonitrile; ii) after completion of the reaction, washing the reaction mixture with an aliphatic hydrocarbon solvent (e.g. hexanes); iii) optionally diluting reaction mixture with an organic solvent (e.g. dichloromethane); iv) washing the mixture with an aqueous solution (e.g. saturated aqueous sodium bicarbonate solution).

(5) We consider the transformation of sulfonium salts into α,β-unsaturated carbonyls to be quite robust and not significantly sensitive to water and air. As is, usage of water containing reagents (i.e. 4-methylmorpholine N-oxide monohydrate) or technical grade solvents or open to air set-ups might be allowed. No problems during the scaling up of the methodology has been experienced (i.e. up to 30 mmol scale), and such operations should be achievable to those skilled in the art.

(6a) As laid out above, the reaction according to the invention is applicable to alkenes which may contain various substituent patterns and different functions, like unsaturated bonds, alkyl or aryl ethers, esters, carboxylic acids, nitriles, alcohols, aldehydes, halogens, pseudohalogens, amides, imides and group(s), which will not be affected by the reaction.

(6b) A great number of unbiased alpha-olefins (e.g. fatty olefin derivatives) can be used as starting materials in the process according to the present invention. Importantly, in these cases, not only the more thermodynamically stable (E)-α,β-unsaturated aldehydes can be obtained selectively, but it is possible to reverse the selectivity and the elusive (Z)-α,β-unsaturated products are also selectively obtained if needed. The surprising degree of control is enabled primarily by the proper setting of the temperature, reaction time, and choice of the base according to the invention and is less dependent on the substrate of such kind. These important features may be a cause of numerous facts including; i) Surprisingly, Type III reactivity of alkenyl sulfonium salts and synthetic equivalents derived from unbiased alpha-olefins matches the nucleophilic reactivity of amine-oxides (e.g. 4-methylmorpholine N-oxide) so well, that the reaction can be carried out at exceptionally low temperatures and thus the kinetic product (Z)-α,β-unsaturated aldehydes can be selectively obtained; ii) elevated temperatures promote the excess formation of the thermodynamically more stable (E)-α,β-unsaturated aldehydes; iii) (Z)-α,β-unsaturated aldehydes are known to have the ability to isomerize to the more stable (E)-α,β-unsaturated aldehydes over time, especially in the presence of an acid/base/light.

(6c) Biased alpha-olefins might also be starting materials in the process according to the present innovation. However, while there is no significant change in yields, an activated allylic CH bond in the appropriate position (i.e. allylbenzenes) or heteroatom connection (i.e. O-allyl ethers) results in minimal control to switch E/Z selectivity. This is not surprising as it is known that the possibility for conjugation, even more, stabilizes the (E)-α,β-unsaturated aldehydes. Thus, this observation is substrate-driven and characteristic of some biased olefins.

(6d) Disubstituted olefins (i.e. two of R1-R4 in formula II, e.g. R1 and R3 or R1 and R2 or R1 and R4 is other than H) consisting of 1,1-disubstituted olefins (e.g. prop-1-en-2-ylbenzene) and 1,2-disubstituted olefins (e.g. cyclohexene) might also be starting materials in the process according to the present innovation. Here we mention that in the case of 1,2-disubstituted olefin starting materials, better results are obtained with the use of sulfoxide reagents (i.e. dimethyl sulfoxide) instead of amine-oxides. We also discovered, that 1,2-disubstitution enables a previously unknown type of alkenyl sulfonium reactivity (Type IV): reaction as an allylic cation without the double bond shifted (e.g. transforming hex-3-ene into hex-3-en-2-one). The two types of products (via Type III and Type IV reactivity) are both possibly produced in these cases.

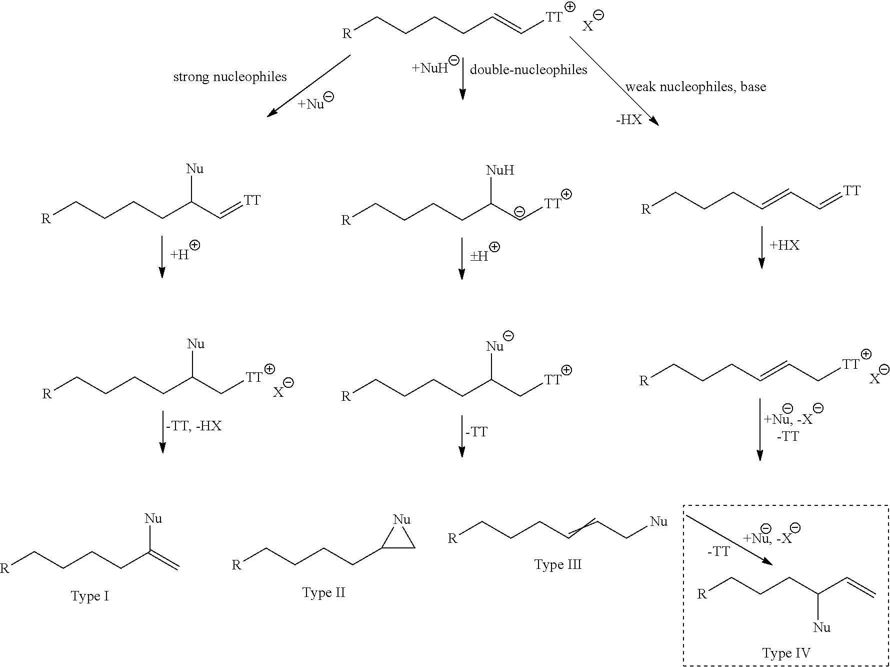

(6e) Trisubstituted olefins (e.g. rose oxide) (i.e. three of R1-R4 in formula II is other than H) might also be starting materials in the process according to the present innovation. Surprisingly, it is possible to significantly shift the selectivity between Type III and Type IV reactivities in this specific case. Namely, selective formation of a Type IV product (i.e. (E)-2-methyl-3-(4-methyltetrahydro-2H-pyran-2-yl)acrylaldehyde) is observed.

(7) According to the present innovation, different sulfonium species consisting of i) alkenyl sulfonium salts; ii) allyl sulfonium salts; iii) bridged dicationic sulfonium salts; and iv) bis-adducts or a mixture thereof all might be suitable intermediates as they provide similar reaction outcomes. In other words, sulfonium salts (i-iv) can be considered synthetically equivalent. This important feature may be a cause of numerous facts including: i) bridged dicationic sulfonium salts and bis-adducts are known to be able to be transformed into the corresponding alkenyl sulfonium salts upon exposure to basic conditions; ii) the conditions of the present method are able to appropriately facilitate these transformations; iii) alkenyl sulfonium salts and allyl sulfonium salts are both productive intermediates and are able to give rise to α,β-unsaturated carbonyl products. Here we mention, that alkenyl sulfonium salts might react via the intermediacy of allyl sulfonium salts, however, this unproven detail does not repeal the fact that both are productive intermediates. Here we mention, that within the salt several non-nucleophilic or poorly nucleophilic counterions known in the art (e.g. triflate, tetrafluoroborate) might be acceptable.

(8) Activation of an alkene, more specifically, the synthesis of alkenyl sulfonium species or synthetic equivalents may be achieved using various methods known in the art. Preferably, using an olefin according to Formula II, a sulfoxide or sulfide according to Formula IIa or IIb, and an activating agent (e.g. adding carboxylic acid anhydride in the presence of an acid) or oxidizing agent (e.g adding 1-Chloromethyl-4-fluoro-1,4-diazoniabicyclo[2.2.2]octane bis(tetrafluoroborate)), which might be replaced with an activating or oxidizing treatment (e.g. electrochemical oxidation) gives satisfactory results. In some embodiments, this activation (e.g. synthesis of an alkenyl sulfonium salt from an alkene) may be followed by additional functional group interconversions of other functional groups of the sulfonium salt obtained (while maintaining the sulfonium functionality intact) before the contact with the oxidizing mixture to yield α,β-unsaturated carbonyl products.

(9) Capability to further transform the product (either the (E) or the (Z)-α,β-unsaturated aldehyde) obtained according to the present innovation is generally true. A transformation of such kind could be chosen and carried out by a skilled person without difficulty. In some embodiments, this is possible without isolation and significant loss of the existing stereochemical purity. This is enabled by the fact, that only thianthrene is present as a significant component besides the desired product in a typical crude product, which molecule does not interfere with most known reactions. Included are generally known transformations, especially reactions with phosphonium ylides to form (E,Z) or (Z,Z) or (Z,E) or (E,E) conjugated diene pheromones, kairomones, and related compounds.

As described above, a number of conjugated diene derivatives obtained via the methods of the invention can be used as insect pheromones or pheromone precursor materials. The precursor materials and pheromone products include, for example, the compounds listed in Table I. The method can be used for synthesizing one or more of the pheromones listed in Table II.

### Table I

(E,E)-2,4-Decadienal; (E,Z)-2,4-Decadienal; (Z,Z)-2,4-Decadienal; (E,E)-3,5-Decadienyl acetate; (Z,E)-3,5-Decadienyl acetate; (E,E)-1,3-Hexadecadien-1-ol; (E,Z)-4,6-Hexadecadien-1-ol; (E,Z)-4,6-Hexadecadienylacetate; (E,Z)-4,6-Hexadecadienal; (Z,Z)-8,10-Hexadecadienyl acetate; (E,E)-9,11-Hexadecadienal; (E,Z)-9,11-Hexadecadienyl acetate; (E,Z)-9,11-Hexadecadienal; (Z,E)-9,11-Hexadecadienal; (E,E)-2,4-Tetradecadienal; (E,E)-3,5-Tetradecadienyl acetate; (E,Z)-3,5-Tetradecadienyl acetate; (Z,E)-3,5-Tetradecadienyl acetate; (E,E)-8,10-Tetradecadien-1-ol; (E,E)-8,10-Tetradecadienyl acetate; (E,E)-8,10-Tetradecadienal; (E,Z)-8,10-Tetradecadienyl acetate; (Z,Z)-9,11-Hexadecadienal; (E,E)-10,12-Hexadecadien-1-ol; (E,E)-10,12-Hexadecadienyl acetate; (E,E)-10,12-Hexadecadienal; (E,Z)-10,12-Hexadecadien-1-ol; (E,Z)-10,12-Hexadecadienyl acetate; (E,Z)-10,12-Hexadecadienal; (Z,E)-10,12-Hexadecadienyl acetate; (Z,E)-10,12-Hexadecadienal; (Z,Z)-10,12-Hexadecadienal; (E,E)-11,13-Hexadecadien-1-ol; (E,E)-11,13-Hexadecadienyl acetate; (E,E)-11,13-Hexadecadienal; (E,Z)-11,13-Hexadecadien-1-ol; (E,Z)-11,13-Hexadecadienyl acetate; (E,Z)-11,13-Hexadecadienal; (Z,E)-11,13-Hexadecadien-1-ol; (Z,E)-11,13-Hexadecadienyl acetate; (Z,E)-11,13-Hexadecadienal; (Z,Z)-11,13-Hexadecadien-1-ol; (Z,Z)-11,13-Hexadecadienyl acetate; (Z,Z)-11,13-Hexadecadienal; (E,Z)-3,5-Dodecadienyl acetate; (Z,E)-3,5-Dodecadienyl acetate; (E,E)-5,7-Dodecadien-1-ol; (E,E)-5,7-Dodecadienyl acetate; (E,Z)-5,7-Dodecadien-1-ol; (E,Z)-5,7-Dodecadienyl acetate; (E,Z)-5,7-Dodecadienal; (Z,E)-5,7-Dodecadien-1-ol; (Z,E)-5,7-Dodecadienyl acetate; (E,Z)-8,10-Tetradecadienal; (Z,E)-8,10-Tetradecadien-1-ol; (Z,E)-8,10-Tetradecadienyl acetate; (Z,Z)-8,10-Tetradecadienal; (E,E)-9,11-Tetradecadienyl acetate; (E,Z)-9,11-Tetradecadienyl acetate; (Z,E)-9,11-Tetradecadien-1-ol; (Z,E)-9,11-Tetradecadienyl acetate; (Z,E)-9,11-Tetradecadienal; (Z,Z)-9,11-Tetradecadien-1-ol; (Z,Z)-9,11-Tetradecadienyl acetate; (Z,Z)-9,11-Tetradecadienal; (E,E)-10,12-Tetradecadien-1-ol (E,E)-10,12-Tetradecadienyl acetate; (E,E)-10,12-Tetradecadienal; (E,Z)-10,12-Tetradecadienyl acetate; (Z,E)-10,12-Tetradecadienyl acetate; (Z,Z)-10,12-Tetradecadien-1-ol; (Z,Z)-8,10-Heptadecadien-1-ol; (Z,E)-5,7-Dodecadienal; (Z,Z)-5,7-Dodecadienyl acetate; (Z,Z)-5,7-Dodecadienal; (E,E)-7,9-Dodecadienyl acetate; (E,Z)-7,9-Dodecadien-1-ol; (E,Z)-7,9-Dodecadienyl acetate; (E,Z)-7,9-Dodecadienal; (Z,E)-7,9-Dodecadien-1-ol; (Z,E)-7,9-Dodecadienyl acetate; (Z,Z)-7,9-Dodecadien-1-ol; (Z,Z)-7,9-Dodecadienyl acetate; (E,E)-8,10-Dodecadien-1-ol; (E,E)-8,10-Dodecadienyl acetate; (E,E)-8,10-Dodecadienal; (E,Z)-8,10-Dodecadien-1-ol; (E,Z)-8,10-Dodecadienyl acetate; (E,Z)-8,10-Dodecadienal; (Z,E)-8,10-Dodecadien-1-ol; (Z,E)-8,10-Dodecadienyl acetate; (Z,E)-8,10-Dodecadienal; (Z,Z)-8,10-Dodecadien-1-ol; (Z,Z)-8,10-Dodecadienyl acetate; (E,E)-8,10-Pentadecadienyl acetate; (E,Z)-8,10-Pentadecadien-1-ol; (E,Z)-8,10-Pentadecadienyl acetate; (Z,E)-8,10-Pentadecadienyl acetate; (Z,Z)-8,10-Pentadecadienyl acetate; (E,Z)-9,11-Pentadecadienal; (Z,Z)-9,11-Pentadecadienal; (Z,Z)-11,13-Octadecadienal; (Z,Z)-13,15-Octadecadienal;

### Table II

(4E,6Z)-hexadeca-4,6-dien-1-ol; (4E,6Z)-hexadeca-4,6-dien-1-yl acetate; (4E,6Z)-hexadeca-4,6-dienal; (2E,4Z)-deca-2,4-dienal; (9E,11Z)-hexadeca-9,11-dien-1-yl acetate; (9E,11Z)-hexadeca-9,11-dienal; (10E,12Z)-hexadeca-10,12-dien-1-ol; (10E,12Z)-hexadeca-10,12-dien-1-yl acetate; (10E,12Z)-hexadeca-10,12-dienal; (3E,5Z)-tetradeca-3,5-dien-1-yl acetate; (11E,13Z)-hexadeca-11,13-dien-1-ol; (11E,13Z)-hexadeca-(11E,13Z)-dien-1-yl acetate; (8E,10Z)-tetradeca-8,10-dien-1-yl acetate; (8E,10Z)-tetradeca-8,10-dienal; (9E,11Z)-tetradeca-9,11-dien-1-yl acetate; (3E,5Z)-dodeca-3,5-dien-1-yl acetate; (5E,7Z)-dodeca-5,7-dien-1-ol; (5E,7Z)-dodeca-5,7-dien-1-yl acetate; (5E,7Z)-dodeca-5,7-dienal; (10E,12Z)-tetradeca-10,12-dien-1-yl acetate; (7E,9Z)-dodeca-7,9-dien-1-ol; (7E,9Z)-dodeca-7,9-dien-1-yl acetate; (7E,9Z)-dodeca-7,9-dienal; (8E,10Z)-dodeca-8,10-dien-1-ol; (8E,10Z)-dodeca-8,10-dien-1-yl acetate; (8E,10Z)-dodeca-8,10-dienal; (8E,10Z)-pentadeca-8,10-dien-1-ol; (8E,10Z)-pentadeca-8,10-dien-1-yl acetate; or (9E,11Z)-pentadeca-9,11-dienal.

### EXAMPLES

### Optimization Studies (Examples 1-6)

### Summarizing the Scope of Alkenes (Example 7)

### Summarizing the Synthesis of Sulfonium Species (Examples 8-13)

### Summarizing the Synthesis of Conjugated Diene Pheromones, Kairomones, and Related Compounds (Examples 14-20)

### Optimization Studies (Examples 1-6)

### Example 1

Optimization of the oxygen source
method A: general conditions

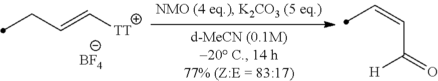

| | deviation:oxygen source | effect | NMR yield (%) (Z/E) |
|---|---|---|---|
| 1 | DIPEANO | complex mixture | 0 (n.d.) |
| 2 | PICNO | complex mixture | 4 (n.d.) |
| 3 | TMANO | enhanced byproduct formation | 39 (83:17) |
| 4 | NMO (1.0 eq.) | enhanced byproduct formation | 59 (88:12) |
| 5 | DMSO, 25° C. [method B] | complex mixture | 44 (68:32) |

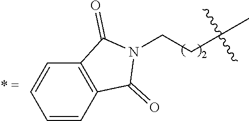

### Example 2

Optimization of the base
method A: general conditions

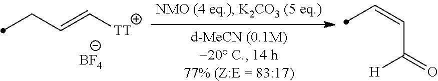

| | deviation:base | effect | NMR yield (%) (Z/E) |
|---|---|---|---|
| 1 | DBU | alkylation of the base | 0 |
| 2 | 2,6-Lutidine | moderatly enhanced B | 74 (82:18) |
| 3 | TMP | moderatly enhanced B | 70 (80:20) |
| 4 | DIPEA | moderatly enhanced B | 69 (87:13) |
| 5 | NaHCO₃ | not significant | 76 (86:14) |
| 6 | K₃PO₄ | not significant | 63 (80:20) |

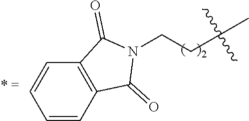

method B: general conditions

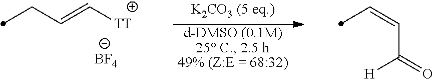

| | | | NMR yield |
|---|---|---|---|
| | Deviation:base | effect | (%) (Z/E) |
| 1 | Cs₂CO₃ | significant decomposition | 0 (n.d.) |
| 2 | DBU | only byproduct formation | 0 (n.d.) |
| 3 | TMP | significant byproduct formation | 26 (70:30) |
| 4 | DIPEA, 16 h | slower conversion | 46 (78:22) |
| 5 | NaHCO₃, 16 h | slower conversion | 52 (70:30) |
| 6 | 2,6-Lutidine | mostly intact | 0 (n.d.) |
| 7 | N,N-dimethylaniline | mostly intact | traces (n.d.) |

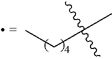

### Example 3

Optimization of the solvent
method A: general conditions

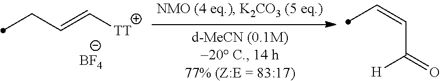

| | deviation:solvent | effect | NMR yield (%) (Z/E) |
|---|---|---|---|
| 1 | d-DCM | enhanced byproduct formation | 53 (72:28) |
| 2 | d-THF | not significant | 71 (81:21) |
| 3 | d-Acetone | not significant | 75 (82:18) |
| 4 | d-Toluene | some dissolution issues | 72 (76:24) |

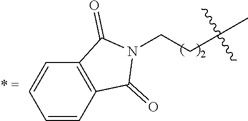

### Example 4

Optimization of temperature and reaction time; synthesis of the (E) isomer
method A: general conditions

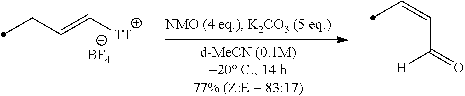

| | | | NMR yield |
|---|---|---|---|
| | deviation:temperature/time | effect | (%) (Z/E) |
| 1 | 0° C., 4 h | enhanced E formation | 77 (77:24) |
| 2 | 25° C., 90 min | enhanced E formation | 73 (49:51) |
| 3 | 25° C., 14 h | isomerization | 56 (2:98) |
| 4 | NaHCO₃, 50° C., 14 h | optimal for E formation | 79 (1:99) |

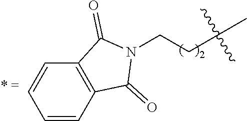

### Example 5

Work-up and isolation
method A: general conditions

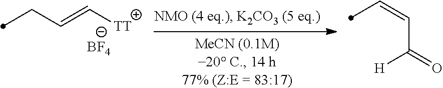

| | deviation:isolation | effect | isolated yield (%) (Z/E) |
|---|---|---|---|
| 1 | column chromatography | some isomerization | 72% (71:29) |
| 2 | extractions | no additional | 70% (81:19) |
| | | isomerization | [95% NMR pur.] |

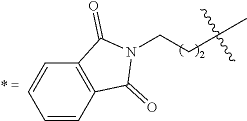

### Modified Work Up of Method A—Extractions (0.4 Mmol)

When analysis by TLC indicated full conversion of the reaction, the mixture was filtered, and the filter cake was washed with acetonitrile (25 mL). The washing solution was recombined with the filtrate. The filtrate was washed with hexanes (5×60 mL) and then diluted with dichloromethane (120 mL). This combined solution was washed with saturated aqueous sodium bicarbonate (2×60 mL) and brine (60 mL) and dried over sodium sulfate. The dried solution was filtered, and the filtrate was concentrated under reduced pressure to yield unsaturated aldehydes.

### Example 6

Testing robustness
method A: general conditions

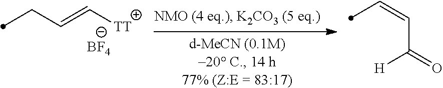

| | | | change in |
|---|---|---|---|
| | deviation | effect | NMR yield (%) |
| 1 | water added (10 equiv) | not significant | <5% |
| 2 | open to air | not significant | <5% |
| 3 | triflate counterion | not significant | <5% |
| 4 | K₂CO₃ neither well powdered, | not significant | <5% |
| | nor oven dried | | |

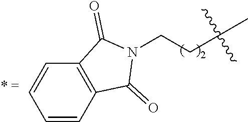

### Summarizing the Scope of Alkenes (Example 7)

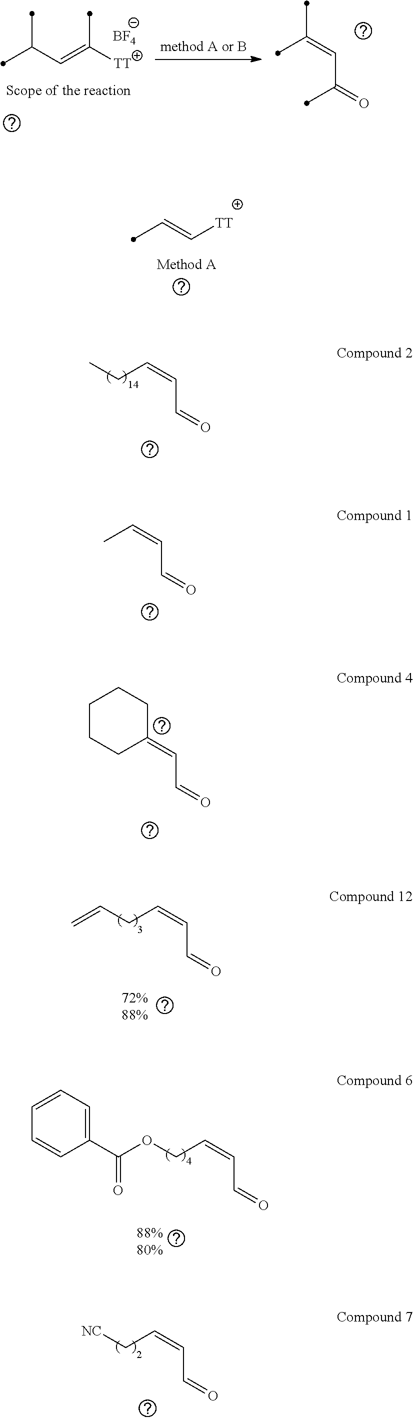

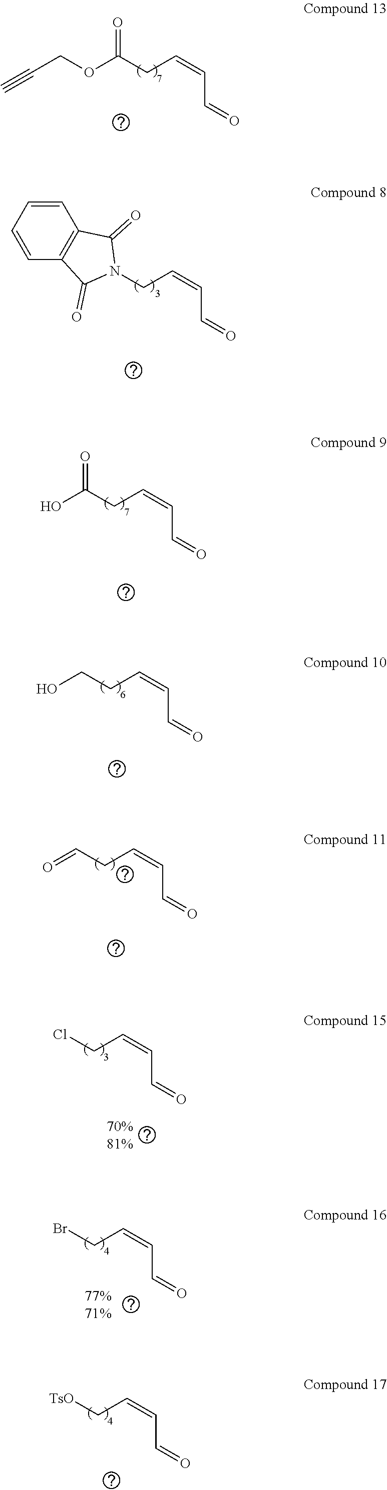

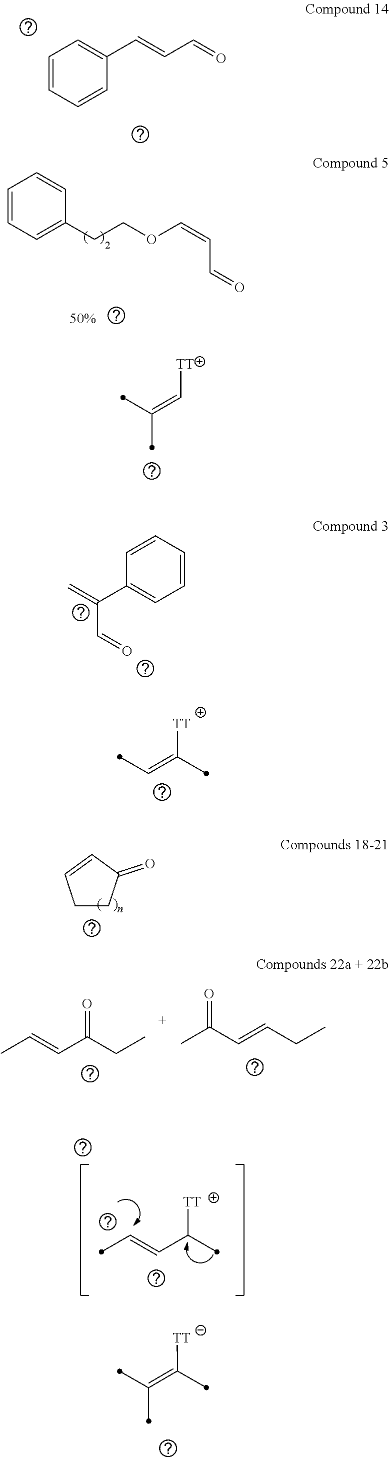

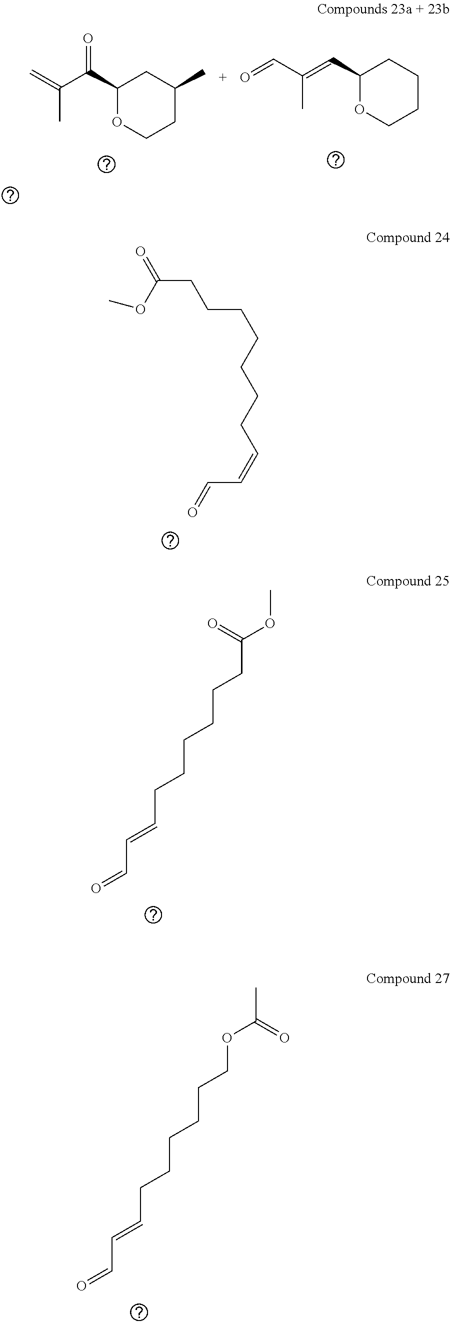

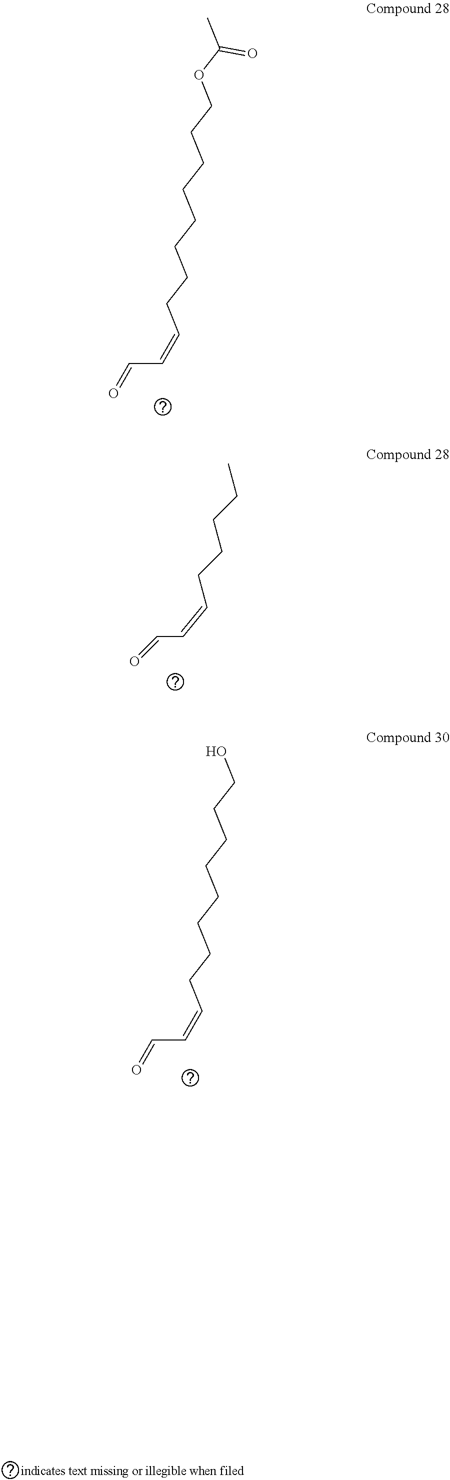

### Example 7

### Method A (Ganem-Like)

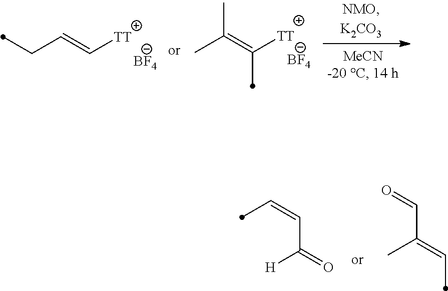

A suspension of N-methylmorpholine N-oxide (NMO, 187 mg, 1.60 mmol, 4.00 equiv) and well powdered, oven-dried potassium carbonate (276 mg, 2.00 mmol, 5.00 equiv) in anhydrous acetonitrile (3.0 mL) was cooled to −20 °C (±2 °C, internal temperature) and stirred at this temperature for 30 min. [Notes 1,2] Then, a solution of alkenyl thianthren-5-ium tetrafluoroborate (0.400 mmol, 1 equiv) in anhydrous acetonitrile (1.0 mL) was added dropwise while maintaining the internal temperature below −18 °C [Note 3] The reaction mixture was stirred at this temperature for 14 h when analysis by TLC indicated full conversion. [Notes 4,5] Then, saturated aqueous sodium bicarbonate solution (30 mL) and dichloromethane (30 mL) were added subsequently. The layers were separated, and the organic phase was dried over sodium sulfate. [Note 6] The dried solution was filtered, and the filtrate was concentrated under reduced pressure. [Note 7] The residue was purified by flash column chromatography on silica gel (gradient elution using ethyl acetate and hexanes) to yield the unsaturated aldehydes.

### Note 8

### Method B (Kornblum-Like)

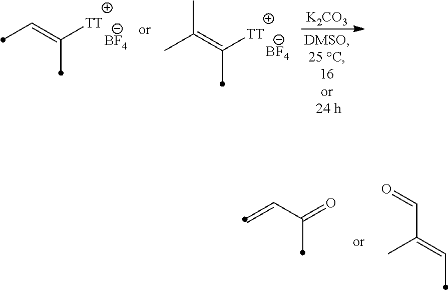

A suspension of well powdered, oven-dried potassium carbonate (276 mg, 2.00 mmol, 5.00 equiv) in anhydrous dimethyl sulfoxide (3.0 mL) was stirred at 25 °C for 5 min. Then, a solution of alkenyl thianthren-5-ium tetrafluoroborate (0.400 mmol, 1 equiv) in anhydrous dimethyl sulfoxide (1.0 mL) was added dropwise and the reaction mixture was stirred at this temperature for 14-24 h when analysis by TLC indicated full conversion. [Notes 3-5] Then, saturated aqueous sodium bicarbonate solution (30 mL) and diethyl ether (30 mL) were added subsequently. The layers were separated, and the aqueous phase was extracted with diethyl ether (2×30 mL). The combined organic layers were washed with brine (3×30 mL) and dried over sodium sulfate. The dried solution was filtered, and the filtrate was concentrated under reduced pressure. The residue was purified by flash column chromatography on silica gel (gradient elution using ethyl acetate and hexanes) to yield the unsaturated carbonyls.

### Note 8

[Note 1]: A low-temperature thermostat was used to control cooling bath temperature.
[Note 2]: This preliminary stirring ensures that the internal temperature stabilizes as initial overcooling to ca. −24 °C may occur due to the endothermic dissolution of K₂CO₃.
[Note 3]: Dissolution and addition was performed in two steps (0.75 mL+0.25 mL wash) to secure complete transfer.
[Note 4]: The reaction mixture has a characteristic appearance on TLC, as demonstrated below by a typical example.
[Note 5]: At this point, NMR yield was measured as follows: trifluorotoluene or ethylene carbonate was added as an internal standard to the reaction mixture and a sample of it (0.1 mL) was diluted with deuterated chloroform (0.8 mL) and filtered. The yield and Z:E ratio (if relevant) were determined by ¹H NMR spectroscopy using solvent suppression.
[Note 6]: This step eliminates most of the excess NMO and N-alkylated by-products. In case of purification by column chromatography, no more washings are required, however, if using the crude mixture, additional washing with saturated aqueous sodium bicarbonate solution (2×30 mL) is recommended.
[Note 7]: To avoid isomerization of (Z)-aldehydes, the water bath temperature was set to 30 °C
[Note 8]: Washing with pure hexanes until the by-product thianthrene elutes, then switching to gradient elution is a practical and effective procedure in most cases. Please see further details at specific substrates.

### Compound 1 ((Z)-pent-2-enal)

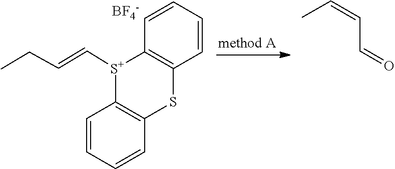

Following method A without any modifications. NMR yield: 52% (Z:E=88:12). No isolated yield was determined due to the volatility of the product. Spectral properties were in accordance with those reported in the literature. (ref xiv)

### Compound 2 ((Z)-octadec-2-enal)

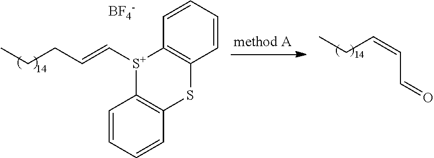

Following method A without any modifications. NMR yield: 70% (Z:E=84:16). Purification by flash column chromatography on flash silica gel (0% ethyl acetate in hexanes grading to 20% ethyl acetate in hexanes) afforded 71 mg (67%, Z:E=79:21) of the title compound 2.

Physical State: colorless oil.

### Z

¹H-NMR (499.64 MHz, CDCl₃): δ=10.07 (d, J=8.1 Hz, 1H), 6.62 (dt, J=11.1, 8.2 Hz, 1H), 5.95 (ddt, J=11.1, 8.1, 1.5 Hz, 1H), 2.60 (qd, J=7.6, 1.5 Hz, 2H), 1.50 (p, J=7.2 Hz, 2H), 1.39-1.17 (m, 24H), 0.87 ppm (t, J=6.9 Hz, 3H).

¹³C-NMR (125.65 MHz, CDCl₃): δ=190.8, 153.3, 130.2, 31.9, 29.67, 29.66, 29.64, 29.63, 29.62, 29.58, 29.5, 29.33, 29.32, 29.2, 29.1, 28.0, 22.7, 14.1 ppm.

### E

¹H-NMR (499.64 MHz, CDCl₃): δ=9.50 (d, J=7.9 Hz, 1H), 6.84 (dt, J=15.5, 6.8 Hz, 1H), 6.11 (ddt, J=15.6, 7.9, 1.5 Hz, 1H), 2.37-2.27 (m, 2H), 1.50 (p, J=7.2 Hz, 2H), 1.41-1.18 (m, 24H), 0.87 ppm (t, J=6.9 Hz, 3H).

¹³C-NMR (125.65 MHz, CDCl₃): δ=194.0, 158.9, 133.0, 32.7, 29 (10 C overlapped with major component) 29.1, 27.8, 22.7, 14.1 ppm.

HRMS (ESI): [M+H]⁺ calcd. for [C₁₈H₃₅O]⁺ 267.2682, found 267.2685.

TLC: Rf=0.50 (10% ethyl acetate in hexanes, CAM).

### Compound 3 (2-phenylacrylaldehyde)

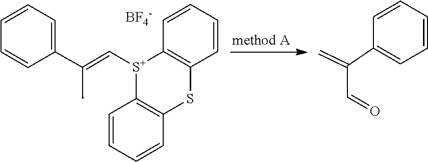

Following method A with the following modifications: starting from the 1:1 mixture of allyl and vinyl isomers of compound 3-TT; temperature at 0 °C; reaction time: 7 h. NMR yield: 58%. Purification by flash column chromatography on flash silica gel (0% ethyl acetate in hexanes grading to 20% ethyl acetate in hexanes) afforded 27 mg (51%) of the title compound 3.

Physical State: yellow oil.

TLC: Rf=0.40 (10% ethyl acetate in hexanes, CAM).

Spectral properties were in accordance with those reported in the literature. (ref xv)

### Compound 4 (2-cyclohexylideneacetaldehyde)

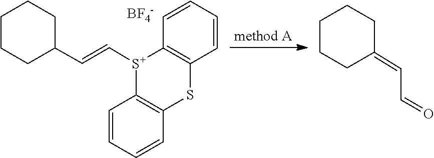

Following method A with the following modifications: temperature at 0 °C NMR yield: 77%. Purification by flash column chromatography on flash silica gel (0% ethyl acetate in hexanes grading to 20% ethyl acetate in hexanes) afforded 32 mg (64%) of the title compound 4.

Physical State: colorless oil.

TLC: Rf=0.30 (10% ethyl acetate in hexanes, CAM).

Spectral properties were in accordance with those reported in the literature. (ref xvi)

### Compound 5 ((Z)-3-(3-phenylpropoxy)acrylaldehyde)

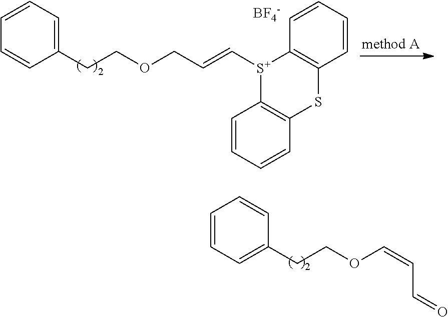

Following method A with the following modifications: starting from the mixture of regioisomeric thianthren-5-ium salts 5-TT. NMR yield: 50% (Z:E=44:56). Unfortunately, it was not possible to isolate the title compound in its pure form due to enhanced instability. Purification by flash column chromatography on flash silica gel (0% ethyl acetate in hexanes grading to 20% ethyl acetate in hexanes) afforded the title compound alongside with 3-phenylpropan-1-ol. Spectral properties were determined from this mixture.

Physical State: colorless oil.

### Z

¹H-NMR (499.64 MHz, CDCl₃): δ=10.06 (d, J=8.4 Hz, 1H), 7.33-7.27 (m, 2H), 7.24-7.16 (m, 3H), 6.93 (d, J=6.3 Hz, 1H), 5.11 (dd, J=8.3, 6.3 Hz, 1H), 4.02 (t, J=6.4 Hz, 2H), 2.78-2.74 (m, 2H), 2.07 ppm (qt, J=7.3, 6.4 Hz, 2H).

¹³C-NMR (125.65 MHz, CDCl₃): δ=189.2, 163.4, 140.5, 128.6, 128.4, 126.3, 108.7, 74.6, 31.6, 31.0 ppm.

### E

¹H-NMR (499.64 MHz, CDCl₃): δ=9.37 (d, J=7.9 Hz, 1H), 7.37 (d, J=12.8 Hz, 1H), 7.33-7.27 (m, 2H), 7.24-7.16 (m, 3H), 5.59 (dd, J=12.7, 7.9 Hz, 1H), 3.93 (t, J=6.3 Hz, 2H), 2.78-2.74 (m, 2H), 2.07 ppm (qt, J=7.3, 6.4 Hz, 2H).

¹³C-NMR (125.65 MHz, CDCl₃): δ=191.2, 170.4, 140.5, 128.6, 128.4, 126.3, 110.2, 70.7, 31.7, 30.2 ppm.

HRMS (ESI): [M+H]⁺ calcd. for [C₁₂H₁₅O]⁺ 191.1067, found 191.1067.

TLC: Rf=0.35 (33% ethyl acetate in hexanes, CAM).

### Compound 6 ((Z)-7-oxohept-5-en-1-yl benzoate)

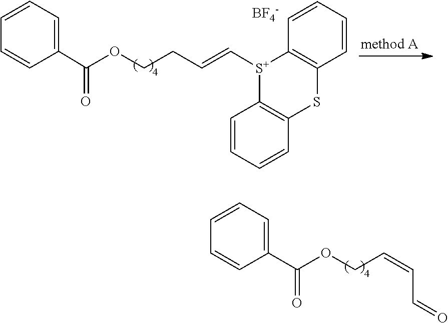

Following method A without any modifications. NMR yield: 65% (Z:E=83:17). Purification by flash column chromatography on flash silica gel (0% ethyl acetate in hexanes grading to 25% ethyl acetate in hexanes) afforded 56 mg (60%, Z:E=78:22) of the title compound 6.

Physical State: colorless oil.

### Z

¹H-NMR (499.64 MHz, CDCl₃): δ=10.08 (d, J=7.9 Hz, 1H), 8.03 (d, J=7.1 Hz, 2H), 7.55 (d, J=7.3 Hz, 1H), 7.44 (t, J=7.7 Hz, 2H), 6.61 (dt, J=11.2, 8.1 Hz, 1H), 5.99 (ddt, J=11.2, 7.9, 1.6 Hz, 1H), 4.36 (t, J=6.5 Hz, 2H), 2.70 (qd, J=7.6, 1.6 Hz, 2H), 1.84 (dq, J=15.2, 6.6 Hz, 2H), 1.69 ppm (tt, J=9.9, 6.5 Hz, 2H).

¹³C-NMR (125.65 MHz, CDCl₃): δ=190.6, 166.5, 152.1, 133.3, 132.9, 130.5, 129.5, 128.4, 64.3, 28.3, 27.6, 25.8 ppm.

### E

¹H-NMR (499.64 MHz, CDCl₃): δ=9.51 (d, J=7.8 Hz, 1H), 8.03 (dd, J=8.3, 1.4 Hz, 2H), 7.55 (d, J=7.3 Hz, 1H), 7.44 (t, J=7.7 Hz, 2H), 6.85 (dt, J=15.6, 6.7 Hz, 1H), 6.15 (ddt, J=15.7, 7.8, 1.5 Hz, 1H), 4.35 (t, J=6.5 Hz, 2H), 2.42 (qd, J=7.4, 1.4 Hz, 2H), 1.84 (dq, J=15.2, 6.6 Hz, 2H), 1.69 ppm (tt, J=9.9, 6.5 Hz, 2H).

¹³C-NMR (125.65 MHz, CDCl₃): δ=193.8, 166.5, 157.5, 133.3, 132.9, 130.2, 129.5, 128.4, 64.3, 32.2, 28.3, 24.4 ppm.

HRMS (ESI): [M+H]⁺ calcd. for [C₁₄H₁O₃]⁺ 233.1172, found 233.1173.

TLC: Rf=0.50 (25% ethyl acetate in hexanes, CAM).

### Compound 7 ((Z)-6-oxohex-4-enenitrile)

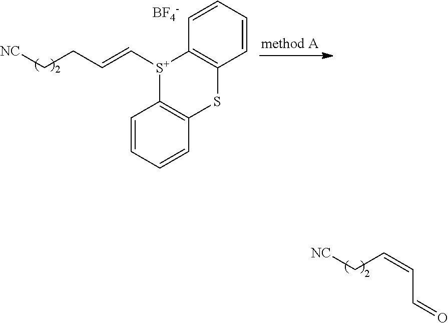

Following method A without any modifications. NMR yield: 55% (Z:E=52:48). Purification by flash column chromatography on flash silica gel (0% ethyl acetate in hexanes grading to 50% ethyl acetate in hexanes) afforded 22 mg (50%, Z:E=43:57) of the title compound 7.

Physical State: colorless oil.

### Z

¹H-NMR (499.64 MHz, CDCl₃): δ=10.01 (d, J=6.7 Hz, 1H), 6.51 (dt, J=11.1, 7.9 Hz, 1H), 6.15 (ddt, J=11.2, 6.7, 1.6 Hz, 1H), 2.96 (qd, J=7.2, 1.5 Hz, 2H), 2.61-2.53 ppm (m, 2H).

¹³C-NMR (125.65 MHz, CDCl₃): δ=189.8, 145.6, 131.7, 118.0, 24.0, 17.0 ppm.

### E

¹H-NMR (499.64 MHz, CDCl₃): δ=9.55 (d, J=7.6 Hz, 1H), 6.80 (dt, J=15.8, 6.5 Hz, 1H), 6.21 (ddt, J=15.8, 7.5, 1.6 Hz, 1H), 2.70 (qd, J=7.1, 1.4 Hz, 2H), 2.60-2.54 ppm (m, 2H).

¹³C-NMR (125.65 MHz, CDCl₃): δ=192.8, 151.3, 134.5, 118.0, 28.0, 16.0 ppm.

HRMS (ESI): [M+H]⁺ calcd. for [C₆H₈NO]⁺ 110.0600, found 110.0601.

TLC: Rf=0.35 (50% ethyl acetate in hexanes, CAM).

### Compound 8 ((Z)-6-(1,3-dioxoisoindolin-2-yl)hex-2-enal)

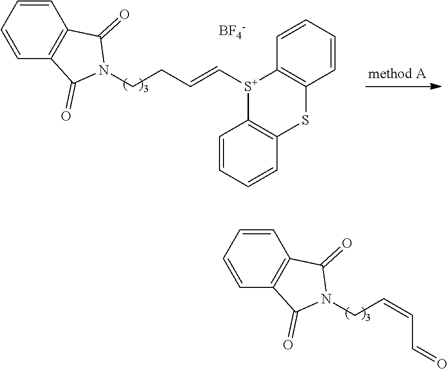

Following method A without any modifications. NMR yield: 75% (Z:E=83:17). Purification by flash column chromatography on flash silica gel (0% ethyl acetate in hexanes grading to 50% ethyl acetate in hexanes) afforded 70 mg (72%, Z:E=71:29) of the title compound 8.

Physical State: colorless oil.

### Z

¹H-NMR (499.64 MHz, CDCl₃): δ=10.04 (d, J=7.8 Hz, 1H), 7.84 (dd, J=5.4, 3.1 Hz, 2H), 7.72 (dd, J=5.5, 3.0 Hz, 3H), 6.59 (dt, J=11.2, 8.0 Hz, 1H), 5.96 (ddt, J=11.2, 7.9, 1.6 Hz, 1H), 3.75 (q, J=7.0 Hz, 2H), 2.68 (qd, J=7.8, 1.6 Hz, 2H), 1.92 ppm (p, J=7.3 Hz, 2H).

¹³C-NMR (125.65 MHz, CDCl₃): δ=190.4, 168.2, 150.9, 134.0, 131.9, 130.6, 123.2, 37.1, 28.1, 25.4 ppm.

### E

¹H-NMR (499.64 MHz, CDCl₃): δ=9.47 (d, J=7.8 Hz, 1H), 7.84 (dd, J=5.4, 3.1 Hz, 2H), 7.72 (dd, J=5.5, 3.0 Hz, 2H), 6.82 (dt, J=15.6, 6.7 Hz, 1H), 5.96 (ddd, J=15.7, 7.7, 1.5 Hz, 1H), 3.75 (q, J=7.0 Hz, 2H), 2.40 (q, J=7.7 Hz, 2H), 1.92 ppm (p, J=7.3 Hz, 2H).

¹³C-NMR (125.65 MHz, CDCl₃): δ=193.5, 168.2, 156.4, 133.4, 131.9, 130.6, 123.2, 37.1, 29.9, 26.7 ppm.

HRMS (ESI): calcd. for C1₄H₁₄NO₃⁺ [M+H]⁺ 244.0968, found 244.0967.

TLC: Rf=0.50 (50% ethyl acetate in hexanes, CAM).

### Compound 9 ((Z)-11-oxoundec-9-enoic acid)

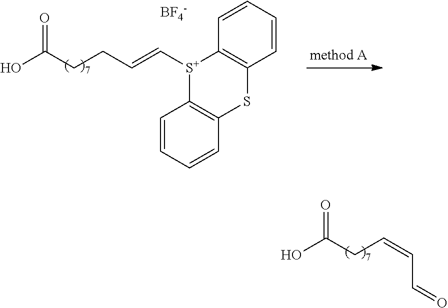

Following method A with the following modifications: aqueous citric acid solution (2 wt. %, 50 mL) was used instead of saturated aqueous sodium bicarbonate solution during the work-up. NMR yield was determined after this modified aqueous work-up from the crude mixture. NMR yield: 46% (Z:E=86:14). Purification by flash column chromatography on flash silica gel (isocratic elution using 10% ethyl acetate and 5% acetic acid in hexanes) afforded 29 mg (37%, Z:E=83:17) of the title compound 9.

Physical State: colorless oil.

### Z

¹H-NMR (499.64 MHz, CDCl₃): δ=10.54 (br s, 1H), 10.06 (d, J=8.1 Hz, 1H), 6.63 (dt, J=11.2, 8.2 Hz, 1H), 5.96 (ddt, J=11.2, 8.1, 1.5 Hz, 1H), 2.75-2.51 (m, 2H), 2.39-2.26 (m, 2H), 1.63 (p, J=7.2 Hz, 2H), 1.50 (p, J=7.3 Hz, 2H), 1.34 ppm (d, J=5.2 Hz, 6H).

¹³C-NMR (125.65 MHz, CDCl₃): δ=191.0, 179.8, 153.5, 130.1, 33.9, 29.0, 28.9, 28.8 (2C), 27.9, 24.5 ppm.

### E

¹H-NMR (499.64 MHz, CDCl₃): δ=10.54 (br s, 1H), 9.49 (d, J=7.9 Hz, 1H), 6.85 (dt, J=15.6, 6.8 Hz, 1H), 6.11 (ddt, J=15.6, 7.9, 1.5 Hz, 1H), 2.75-2.51 (m, 2H), 2.39-2.26 (m, 2H), 1.63 (p, J=7.2 Hz, 2H), 1.50 (p, J=7.3 Hz, 2H), 1.34 ppm (d, J=5.2 Hz, 6H).

¹³C-NMR (125.65 MHz, CDCl₃): δ=194.3, 179.8, 159.0, 132.9, 32.6, 29.0, 28.9, 28.8 (2C), 27.7, 24.5 ppm.

HRMS (ESI): calcd. for C₁H₁₉O₃⁺ [M+H]⁺ 199.1329, found 199.1330.

TLC: Rf=0.30 (10% ethyl acetate and 5% acetic acid in hexanes, CAM).

### Compound 10 ((Z)-10-hydroxydec-2-enal)

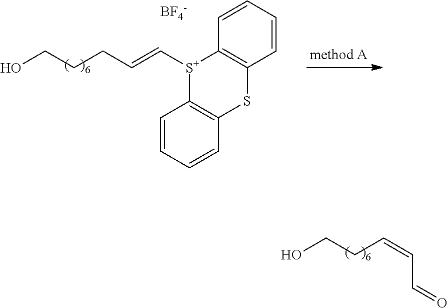

Following method A without any modifications. NMR yield: 71% (Z:E=87:13). Purification by flash column chromatography on flash silica gel (0% ethyl acetate in hexanes grading to 50% ethyl acetate in hexanes) afforded 65 mg (65%, Z:E=82:18) of the title compound 10.

Physical State: colorless oil.

### Z

¹H-NMR (499.64 MHz, CDCl₃): δ=10.06 (d, J=8.1 Hz, 1H), 6.61 (dt, J=11.2, 8.2 Hz, 1H), 6.01-5.86 (m, 1H), 3.63 (t, J=6.6 Hz, 2H), 2.59 (qd, J=7.7, 1.5 Hz, 2H), 1.96 (br s, 1H), 1.55 (t, J=6.8 Hz, 2H), 1.51 (t, J=7.1 Hz, 2H), 1.40-1.32 ppm (m, 6H).

¹³C-NMR (125.65 MHz, CDCl₃): δ=190.9, 153.3, 130.1, 62.8, 32.6, 29.1 (2C), 29.0, 27.9, 25.6 ppm.

### E

¹H-NMR (499.64 MHz, CDCl₃): δ=9.49 (d, J=7.9 Hz, 1H), 6.83 (dt, J=15.6, 6.8 Hz, 1H), 6.10 (ddt, J=15.6, 7.8, 1.5 Hz, 1H), 3.63 (t, J=6.6 Hz, 2H), 2.39-2.25 (qd, J=7.1, 1.0 Hz 2H), 1.55 (t, J=7.0 Hz, 2H), 1.51 (dd, J=11.6, 4.9 Hz, 2H), 1.38-1.31 ppm (m, 6H).

¹³C-NMR (125.65 MHz, CDCl₃): δ=194.1, 158.8, 133.0, 62.8, 32.6, 29.1 (2C), 29.0, 27.7, 25.6 ppm.

HRMS (ESI): calcd. for C₁₀H₁₉O₂⁺ [M+H]⁺ 171.1380, found 171.1378.

TLC: Rf=0.35 (50% ethyl acetate in hexanes, CAM)

### Compound 11 ((Z)-dec-2-enedial)

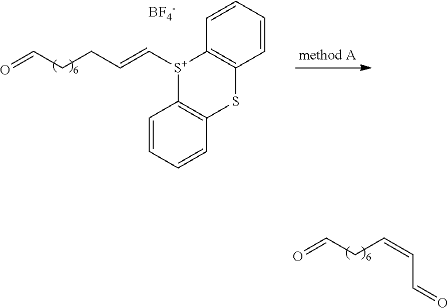

Following method A without any modifications. NMR yield: 73% (Z:E=84:16). Purification by flash column chromatography on flash silica gel (0% ethyl acetate in hexanes grading to 50% ethyl acetate in hexanes) afforded 42 mg (62%, Z:E=79:21) of the title compound 11.

Physical State: colorless oil.

### Z

¹H-NMR (499.64 MHz, CDCl₃): δ=10.05 (d, J=8.0 Hz, 1H), 9.74 (t, J=1.7 Hz, 1H), 6.59 (dt, J=11.2, 8.2 Hz, 1H), 5.94 (ddt, J=11.2, 8.0, 1.5 Hz, 1H), 2.59 (qd, J=7.6, 1.6 Hz, 2H), 2.41 (td, J=7.3, 1.7 Hz, 2H), 1.62 (p, J=7.3 Hz, 2H), 1.50 (p, J=6.9 Hz, 2H), 1.36 ppm (tt, J=8.1, 4.8 Hz, 4H).

¹³C-NMR (125.65 MHz, CDCl₃): δ=202.4, 190.7, 152.9, 130.2, 43.7, 28.9, 28.8 (2C), 27.8, 21.8 ppm.

### E

¹H-NMR (499.64 MHz, CDCl₃): δ=9.74 (t, J=1.7 Hz, 1H), 9.48 (d, J=7.9 Hz, 1H), 6.82 (dt, J=15.6, 6.8 Hz, 1H), 6.09 (ddt, J=15.6, 7.8, 1.5 Hz, 1H), 2.41 (td, J=7.3, 1.7 Hz, 2H), 2.32 ((td, J=7.4, 1.4 Hz, 2H), 1.62 (p, J=7.3 Hz, 2H), 1.50 (q, J=7.1 Hz, 2H), 1.36 ppm (tt, J=8.1, 4.8 Hz, 4H).

¹³C-NMR (125.65 MHz, CDCl₃): δ=202.4, 193.9, 158.4, 133.0, 43.7, 32.5, 28.8 (2C), 27.5, 21.8 ppm.

HRMS (ESI): calcd. for C₁₀H₇O₂⁺ [M+H]⁺ 169.1223, found 169.1221.

TLC: Rf=0.40 (25% ethyl acetate in hexanes, CAM).

### Compound 12 ((Z)-octa-2,7-dienal)

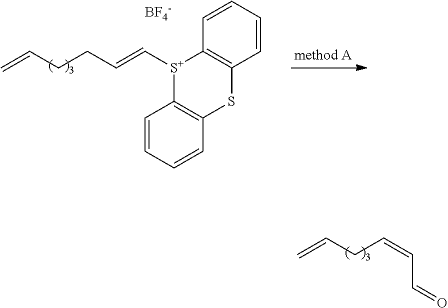

Following method A without any modifications. NMR yield: 72% (Z:E=81:19). Purification by flash column chromatography on flash silica gel (0% ethyl acetate in hexanes grading to 50% ethyl acetate in hexanes) afforded 34 mg (68%, Z:E=79:21) of the title compound 12.

Physical State: colorless oil.

### Z

¹H-NMR (499.64 MHz, CDCl₃): δ=10.07 (d, J=8.0 Hz, 1H), 6.61 (dt, J=11.2, 8.2 Hz, 1H), 5.97 (ddt, J=11.2, 8.1, 1.6 Hz, 1H), 5.79 (ddt, J=17.0, 10.2, 6.7 Hz, 1H), 5.07-5.01 (m, 2H), 2.62 (qd, J=7.8, 1.4 Hz, 2H), 2.13 (p, J=7.4 Hz, 2H), 1.62 ppm (p, J=7.5 Hz, 2H).

¹³C-NMR (125.65 MHz, CDCl₃): δ=190.8, 152.7, 137.6, 130.4, 115.5, 32.9, 28.2, 27.2 ppm.

### E

¹H-NMR (499.64 MHz, CDCl₃): δ=9.51 (d, J=7.9 Hz, 1H), 6.84 (dt, J=15.6, 6.8 Hz, 1H), 6.13 (ddt, J=15.7, 7.9, 1.5 Hz, 1H), 5.79 (ddt, J=17.0, 10.2, 6.7 Hz, 1H), 5.12-4.96 (m, 2H), 2.36 (dt, J=7.4, 1.4 Hz, 2H), 2.13 (p, J=7.4 Hz, 2H), 1.62 ppm (p, J=7.5 Hz, 2H).

¹³C-NMR (125.65 MHz, CDCl₃): δ=193.9, 158.3, 133.2, 130.4, 115.5, 33.0, 32.0, 27.0 ppm.

HRMS (ESI): calcd. for C₈H₁₃O⁺ [M+H]⁺ 125.0961, found 125.0962.

TLC: Rf=0.70 (25% ethyl acetate in hexanes, CAM).

### Compound 13 (prop-2-yn-1-yl (Z)-11-oxoundec-9-enoate)

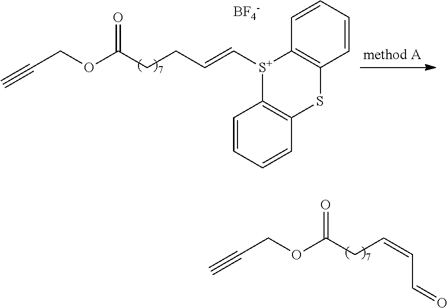

Following method A without any modifications. NMR yield: 67% (Z:E=85:15). Purification by flash column chromatography on flash silica gel (0% ethyl acetate in hexanes grading to 25% ethyl acetate in hexanes) afforded 60 mg (63%, Z:E=78:22) of the title compound 13.

Physical State: colorless oil.

### Z

¹H-NMR (499.64 MHz, CDCl₃): δ=10.06 (d, J=8.0 Hz, 1H), 6.60 (dt, J=11.2, 8.2 Hz, 1H), 5.94 (ddt, J=11.1, 8.0, 1.6 Hz, 1H), 4.66 (d, J=2.5 Hz, 2H), 2.59 (qd, J=7.6, 1.5 Hz, 2H), 2.45 (t, J=2.5 Hz, 1H), 2.34 (t, J=7.5 Hz, 2H), 1.63 (dq, J=9.7, 7.2 Hz, 2H), 1.50 (p, J=7.3 Hz, 2H), 1.33 ppm (pd, J=4.7, 2.8 Hz, 6H).

¹³C-NMR (125.65 MHz, CDCl₃): δ=190.8, 172.8, 153.1, 130.2, 77.8, 74.7, 51.7, 33.9, 29.1, 28.9, 28.8 (2C), 27.9, 24.7 ppm.

### E

¹H-NMR (499.64 MHz, CDCl₃): δ=9.49 (d, J=7.9 Hz, 1H), 6.83 (dt, J=15.5, 6.8 Hz, 1H), 6.10 (ddt, J=15.6, 7.9, 1.5 Hz, 1H), 4.66 (d, J=2.5 Hz, 2H), 2.45 (t, J=2.5 Hz, 1H), 2.35-2.30 (m, J=7.5 Hz, 4H), 1.63 (dq, J=9.7, 7.2 Hz, 2H), 1.50 (p, J=7.3 Hz, 2H), 1.33 ppm (pd, J=4.7, 2.8 Hz, 6H).

¹³C-NMR (125.65 MHz, CDCl₃): δ=194.0, 172.8, 158.6, 133.0, 77.8, 74.7, 51.7, 32.6, 29.1, 28.86, 28.85 (2C), 27.7, 24.7 ppm.

HRMS (ESI): calcd. for C₁₄H₂₁O₃⁺ [M+H]⁺ 237.1485, found 237.1486.

TLC: Rf=0.45 (25% ethyl acetate in hexanes, CAM).

### Compound 14 (Cinnamaldehyde)

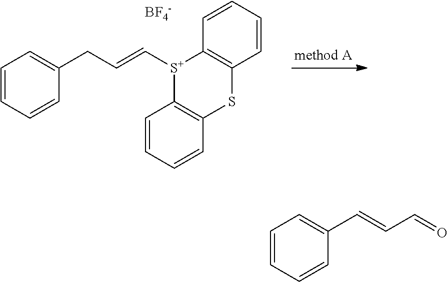 [SMILES: C(=C\[S+]1c2ccccc2Sc2ccccc21)/Cc1ccccc1 ; "BF4-" ; O=C/C=C/c1ccccc1]

Following method A without any modifications. NMR yield: 66% (Z:E=8:92). Purification by flash column chromatography on flash silica gel (0% ethyl acetate in hexanes grading to 25% ethyl acetate in hexanes) afforded 34 mg (64%, E isomer) of the title compound 14.

Physical State: yellow oil.

TLC: Rf=0.50 (25% ethyl acetate in hexanes, CAM).

Spectral properties were in accordance with those reported in the literature. (ref xvii)

### Compound 15 ((Z)-6-chlorohex-2-enal)

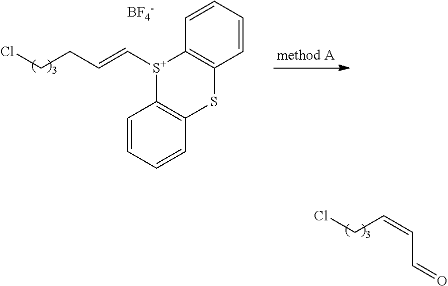

Following method A without any modifications. NMR yield: 70% (Z:E=82:18). Purification by flash column chromatography on flash silica gel (0% ethyl acetate in hexanes grading to 25% ethyl acetate in hexanes) afforded 27 mg (51%, Z:E=67:33) of the title compound 15. Significant yield loss and isomer ratio change were experienced probably due to enhanced and different volatility of the two isomers.

Physical State: colorless oil.

### Z

¹H-NMR (499.64 MHz, CDCl₃): δ=10.09 (d, J=7.8 Hz, 1H), 6.56 (dt, J=11.2, 8.1 Hz, 1H), 6.01 (ddt, J=11.1, 7.9, 1.6 Hz, 1H), 3.57 (td, J=6.4, 4.5 Hz, 2H), 2.79 (tdd, J=8.1, 6.8, 1.6 Hz, 2H), 1.99 ppm (dddd, J=12.8, 8.1, 6.4, 2.2 Hz, 3H).

¹³C-NMR (125.65 MHz, CDCl₃): δ=190.5, 150.3, 131.2, 43.7, 31.5 25.1 ppm.

### E

¹H-NMR (499.64 MHz, CDCl₃): δ=9.52 (d, J=7.8 Hz, 1H), 6.82 (dt, J=15.6, 6.7 Hz, 1H), 6.15 (ddt, J=15.6, 7.7, 1.6 Hz, 1H), 3.57 (td, J=6.3, 4.5 Hz, 2H), 2.52 (dt, J=7.2, 1.4 Hz, 2H), 1.99 (dddd, J=12.8, 8.1, 6.4, 2.2 Hz, 3H).

¹³C-NMR (125.65 MHz, CDCl₃): δ=193.6, 156.0, 133.7, 43.8, 30.5, 29.7 ppm.

HRMS (ESI): calcd. for C₆H₁₀C₁₀⁺ [M+H]⁺ 133.0415, found 133.0414.

TLC: Rf=0.40 (25% ethyl acetate in hexanes, CAM).

### Compound 16 ((Z)-7-bromohept-2-enal)

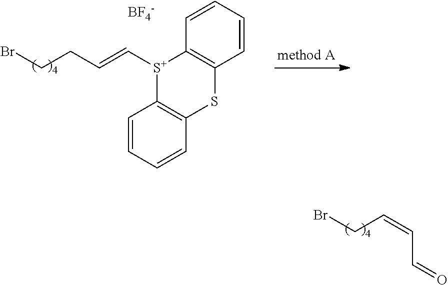

Following method A without any modifications. NMR yield: 77% (Z:E=88:12). Purification by flash column chromatography on flash silica gel (0% ethyl acetate in hexanes grading to 25% ethyl acetate in hexanes) afforded 54 mg (71%, Z:E=84:16) of the title compound 16.

Physical State: colorless oil.

### Z

¹H-NMR (499.64 MHz, CDCl₃): δ=10.06 (d, J=7.9 Hz, 1H), 6.59 (dt, J=11.2, 8.1 Hz, 1H), 5.99 (ddd, J=11.2, 7.8, 1.6 Hz, 1H), 3.42 (t, J=6.6 Hz, 2H), 2.65 (qd, J=7.7, 1.6 Hz, 2H), 1.92 (dt, J=15.1, 6.8 Hz, 2H), 1.68 ppm (p, J=7.5 Hz, 2H).

¹³C-NMR (125.65 MHz, CDCl₃): δ=190.5, 151.8, 130.5, 32.9, 31.9, 27.6, 27.1 ppm.

### E

¹H-NMR (499.64 MHz, CDCl₃): δ=9.51 (d, J=7.8 Hz, 1H), 6.82 (dt, J=15.6, 6.7 Hz, 1H), 6.13 (ddt, J=15.6, 7.8, 1.5 Hz, 1H), 3.42 (td, J=6.6, 2.8 Hz, 2H), 2.38 (qd, J=7.1, 1.5 Hz, 2H), 1.92 (dt, J=15.1, 6.8 Hz, 2H), 1.68 ppm (p, J=7.5 Hz, 2H).

¹³C-NMR (125.65 MHz, CDCl₃): δ=193.7, 157.3, 133.4, 32.0, 31.7, 31.9, 27.6, 26.3 ppm.

HRMS (ESI): calcd. for C7H₁₂BrO⁺ [M+H]⁺ 191.0066, found 191.0065.

TLC: Rf=0.50 (25% ethyl acetate in hexanes, CAM).

### Compound 17 ((Z)-10-oxodec-8-en-1-yl 4-methylbenzenesulfonate)

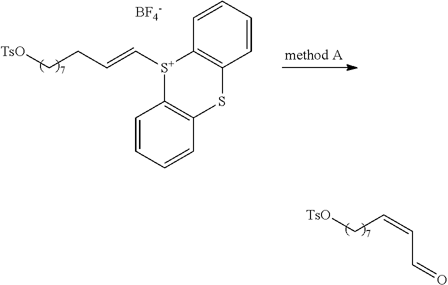

Following method A without any modifications. NMR yield: 84% (Z:E=81:19). Purification by flash column chromatography on flash silica gel (0% ethyl acetate in hexanes grading to 30% ethyl acetate in hexanes) afforded 100 mg (77%, Z:E=79:21) of the title compound 17.

Physical State: colorless oil.

### Z

¹H-NMR (499.64 MHz, CDCl₃): δ=10.05 (d, J=8.0 Hz, 1H), 7.77 (d, J=8.3 Hz, 2H), 7.33 (d, J=8.0 Hz, 2H), 6.59 (dt, J=11.2, 8.2 Hz, 1H), 5.94 (ddt, J=11.2, 8.0, 1.5 Hz, 1H), 4.01 (t, J=6.5 Hz, 2H), 2.57 (qd, J=7.6, 1.5 Hz, 2H), 2.44 (s, 3H), 1.63 (p, J=7.3 Hz, 2H), 1.47 (p, J=7.2 Hz, 2H), 1.36-1.22 ppm (m, 6H).

¹³C-NMR (125.65 MHz, CDCl₃): δ=190.8, 153.0, 144.6, 133.3, 130.2, 129.8, 127.8, 70.5, 29.0, 28.8, 28.7, 28.6, 27.9, 25.2, 21.6 ppm.

### E

¹H-NMR (499.64 MHz, CDCl₃): δ=9.49 (d, J=7.9 Hz, 1H), 7.77 (d, J=8.3 Hz, 2H), 7.33 (d, J=8.0 Hz, 2H), 6.82 (dt, J=15.6, 6.8 Hz, 1H), 6.09 (ddt, J=15.6, 7.9, 1.5 Hz, 1H), 4.01 (t, J=6.5 Hz, 2H), 2.44 (s, 3H), 2.36-2.26 (m, 2H), 1.63 (p, J=7.3 Hz, 2H), 1.47 (p, J=7.2 Hz, 2H), 1.37-1.22 ppm (m, 6H).

¹³C-NMR (125.65 MHz, CDCl₃): δ=193.9, 158.5, 144.6, 133.0, 130.2, 129.8, 127.8, 70.5, 32.5, 29.0, 28.8, 28.7, 28.6, 27.6, 25.2, 21.6 ppm.

HRMS (ESI): calcd. for C₁₇H₂₅O₄S₃⁺ [M+H]⁺ 325.1468, found 325.1464.

TLC: Rf=0.35 (25% ethyl acetate in hexanes, CAM).

### Compound 18 (cyclopent-2-en-1-one)

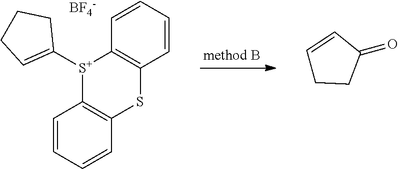

Following method B without any modifications. NMR yield: 33%. No isolated yield was determined due to the volatility of the product. Spectral properties were in accordance with those reported in the literature. (ref xviii)

### Compound 19 (cyclohex-2-en-1-one)

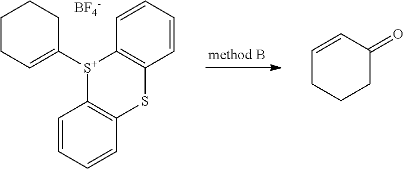

Following method B without any modifications. NMR yield: 61%. No isolated yield was determined due to the volatility of the product. Spectral properties were in accordance with those reported in the literature. (ref xviii)

### Compound 20 (cyclohept-2-en-1-one)

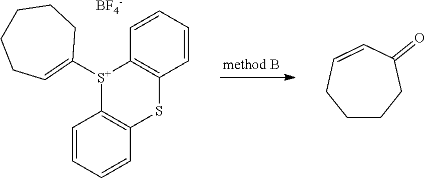 [SMILES: C1=C([S+]2c3ccccc3Sc3ccccc32)CCCCC1 ; "BF4-" ; O=C1C=CCCCC1]

Following method B without any modifications. NMR yield: 57%. No isolated yield was determined due to the volatility of the product. Spectral properties were in accordance with those reported in the literature. (ref xix)

### Compound 21 (cyclooct-2-en-1-one)

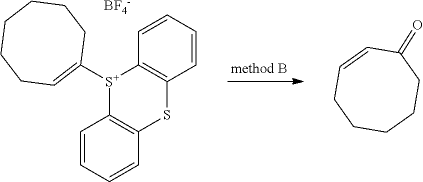 [SMILES: C1=C(/[S+]2c3ccccc3Sc3ccccc32)CCCCCC/1 ; "BF4-" ; O=C1/C=C\CCCCC1]

Following method B without any modifications. NMR yield: 61%. No isolated yield was determined due to the volatility of the product. Spectral properties were in accordance with those reported in the literature. (ref xix)

### Compounds 22a ((E)-hex-3-en-2-one) and 22b ((E)-hex-4-en-3-one)

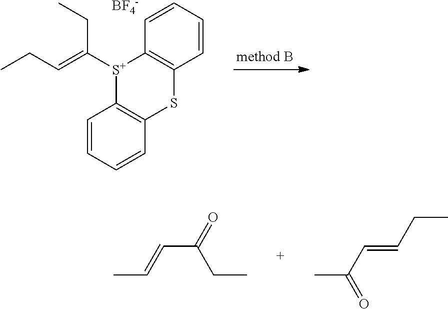

Following method B without any modifications. NMR yield: 41% 22a and 32% 22b. No isolated yield was determined due to the volatility of the products. Spectral properties were in accordance with those reported in the literature. (ref xx)

### Compounds 23a (2-methyl-1-((2R,4S)-4-methyltetrahydro-2H-pyran-2-yl)prop-2-en-1-one) and 23b ((E)-2-methyl-3-((2R,4S)-4-methyltetrahydro-2H-pyran-2-yl) acrylaldehyde)

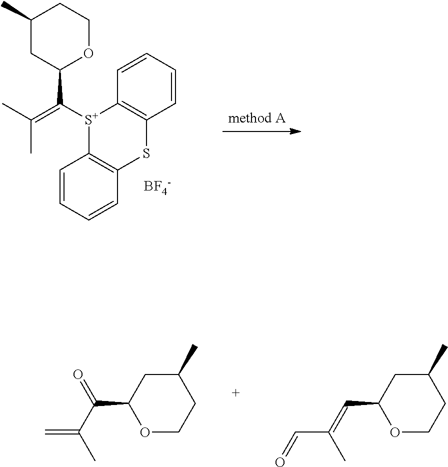

Following method A with the following modifications: temperature at 25 °C NMR yield: 3% 23a and 69% 23b. Purification by flash column chromatography on flash silica gel (0% ethyl acetate in hexanes grading to 15% ethyl acetate in hexanes) afforded 41 mg (61%) of the title compounds 23.

Following method B without any modifications. NMR yield: 4% 23a and 32% 23b.

Unfortunately, it was not possible to isolate adequate amounts of the low yielding 23a on this scale. To afford a small amount of clean analytical sample, the reaction was also carried out and purified on a 1.2 mmol scale using method B.

### Compound 23a

Physical State: colorless oil.

¹H-NMR (499.64 MHz, CDCl₃): δ=5.99 (q, J=0.9 Hz, 1H), 5.83 (dt, J=1.5, 0.7 Hz, 1H), 4.47 (dd, J=11.7, 2.2 Hz, 1H), 4.14 (ddd, J=11.4, 4.7, 1.5 Hz, 1H), 3.50 (ddd, J=12.3, 11.5, 2.2 Hz, 1H), 1.90 (t, J=1.2 Hz, 3H), 1.83 (ddd, J=13.3, 4.0, 2.0 Hz, 1H), 1.71 (tdd, J=9.3, 7.2, 4.5 Hz, 1H), 1.58 (dqd, J=13.4, 3.9, 2.7 Hz, 1H), 1.31 (qd, J=12.3, 4.7 Hz, 1H), 1.18 (dt, J=13.0, 11.7 Hz, 1H), 0.97 (d, J=6.5 Hz, 3H).

¹³C-NMR (125.65 MHz, CDCl₃): δ=199.6, 142.3, 125.4, 79.0, 68.3, 37.6, 34.0, 30.3, 22.2, 18.1 ppm.

HRMS (ESI): calcd. for C₁₀H₁₇O₂⁺ [M+H]⁺ 169.1223, found 169.1223.

TLC: Rf=0.35 (10% ethyl acetate in hexanes, CAM).

### Compound 23b

Physical State: colorless oil.

¹H-NMR (499.64 MHz, CDCl₃): δ=9.38 (s, 1H), 6.37 (dq, J=7.4, 1.3 Hz, 1H), 4.26 (ddd, J=11.4, 7.3, 2.3 Hz, 1H), 4.01 (ddd, J=11.6, 4.6, 1.6 Hz, 1H), 3.47 (ddd, J=12.4, 11.5, 2.2 Hz, 1H), 1.74 (d, J=1.5 Hz, 3H), 1.68 (tdd, J=10.2, 7.2, 5.1 Hz, 1H), 1.63-1.52 (m, 2H), 1.25 (dd, J=12.6, 4.6 Hz, 1H), 1.08 (dt, J=13.1, 11.6 Hz, 1H), 0.95 ppm (d, J=6.6 Hz, 3H).

¹³C-NMR (125.65 MHz, CDCl₃): δ=195.0, 152.9, 138.1, 74.7, 67.8, 38.7, 34.0, 29.9, 22.1, 9.6 ppm.

HRMS (ESI): calcd. for C₁₀H₁₇O₂⁺ [M+H]⁺ 169.1223, found 169.1223.

TLC: Rf=0.25 (10% ethyl acetate in hexanes, CAM).

### Summarizing the Synthesis of Sulfonium Species (Examples 8-13)

### Synthesis of Alkenyl Thianthrenium Salts

Alkenyl thianthren-5-ium salts shown below were reported by Ritter and Shu. (ref xxi) Please see these references for characterization as well as graphical supporting information.

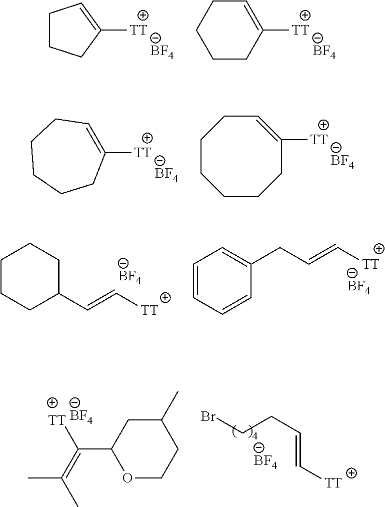

### 1. Figure Known Alkenyl Thianthren-5-Ium Salts

Additional alkenyl thianthren-5-ium salts were synthesized according to slight modifications of these literature known general procedures or specific methods as listed below.

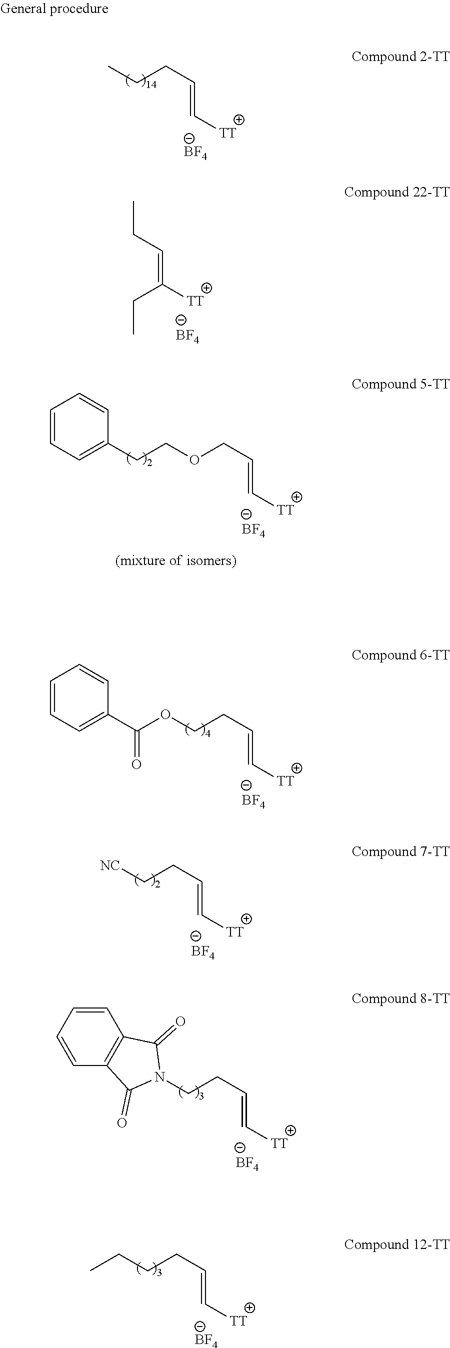

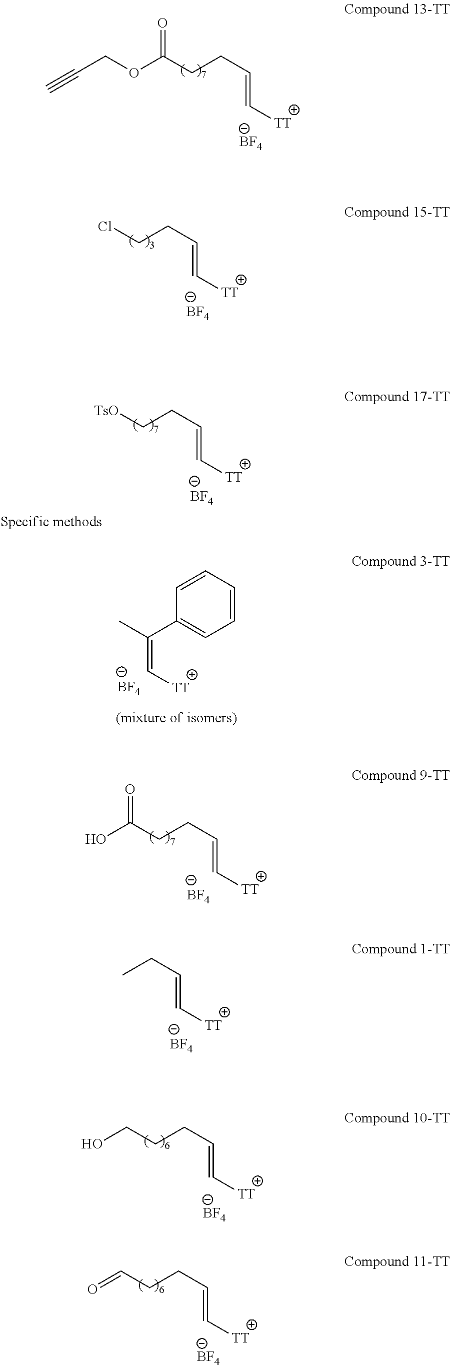

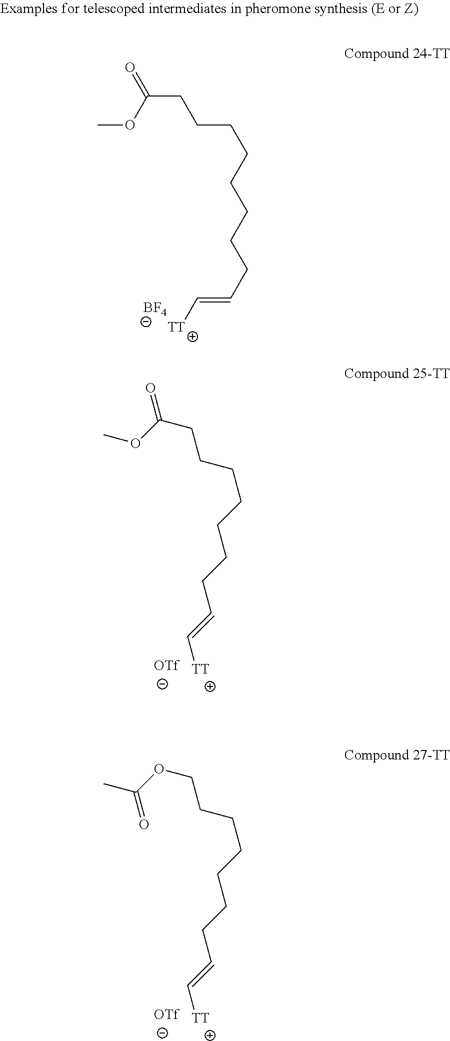

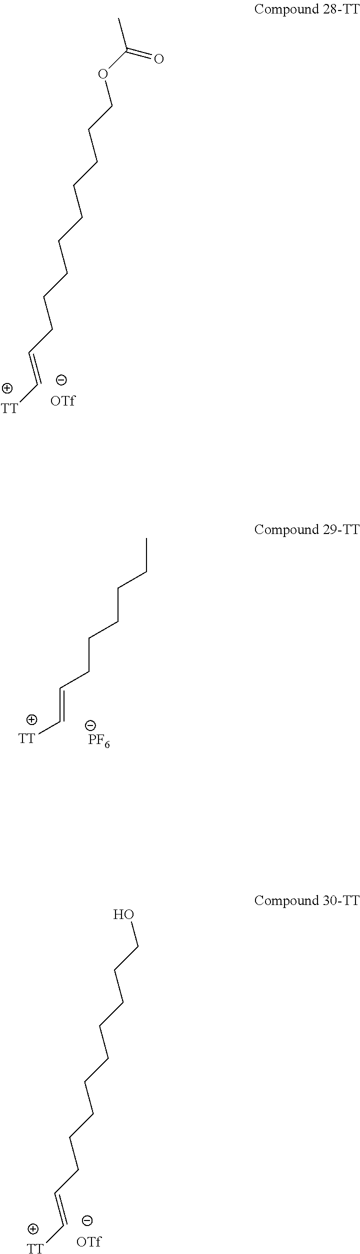

### 2. Figure Previously Unknown Alkenyl Thianthren-5-Ium Salts and Ones Prepared by Modified Procedures

### Example 8

### General Preparation of Alkenyl Thianthren-5-Ium Salts

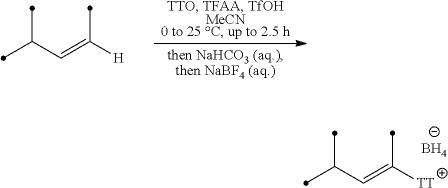

A suspension of alkene (3.15 mmol, 1 equiv) and thianthrene S-oxide (TTO, 754 mg, 3.24 mmol, 1.03 equiv) in anhydrous acetonitrile (10 mL, 0.3 M) was cooled to 0 °C under argon atmosphere. [Note 1] At this temperature, trifluoroacetic anhydride (TFAA, 1.33 mL, 9.45 mmol, 3.00 equiv) and trifluoromethanesulfonic acid (560 μL, 6.30 mmol, 2.00 equiv) were added dropwise consecutively. An immediate color change of the reaction mixture was observed at the beginning of the first addition step and the lilac/blue solution was stirred at 0 °C for a further 1 h. [Note 2] Then, the reaction mixture was warmed to 25 °C and stirred at this temperature for an additional 30 min-1.5 h until analysis by TLC indicated full conversion. [Note 3] At this point, the reaction mixture was concentrated under reduced pressure [Note 4] and subsequently dissolved in dichloromethane (30 mL). Saturated aqueous sodium bicarbonate solution (30 mL) was added, and the two-phase mixture was vigorously stirred at 25 °C for 15 min, whereupon a color change to yellow/brown was observed and the phases were separated. [Notes 5,6] This washing step was repeated one more time, after which aqueous sodium tetrafluoroborate solution (5 wt. %, 50 mL) was added to the organic phase and the mixture was stirred vigorously for 15 min and the phases were separated. This washing step was repeated as well one more time. [Note 7] The layers were separated, and the organic phase was dried over sodium sulfate. The dried solution was filtered, and the filtrate was concentrated under reduced pressure. The residue was purified by flash column chromatography on silica gel (gradient elution using isopropanol and dichloromethane) to yield alkenyl thiantren-5-ium tetrafluoroborate salts as a mixture of E/Z isomers. [Notes 8,9]
[Note 1]: An ice bath was used.
[Note 2]: In the presence of functional groups which are likely to be acetylated by trifluoroacetic anhydride (e.g., alcohols, carboxylic acids), this color change is not immediate but occurs during addition.
[Note 3]: Alkenyl thianthren-5-ium products have a characteristic appearance on TLC, as demonstrated below by a typical example.
[Note 4]: Water jet vacuum pumps were used with gentle heating by a 30 °C water bath.
[Note 5]: This step promotes the formation of alkenyl thianthren-5-ium salts from the precursor dicationic species.
[Note 6]: In some specific cases, this color change was observed before washing with saturated aqueous sodium bicarbonate solution, however, this did not affect the outcome of the reaction.
[Note 7]: This step completes the counterion exchange. Completeness can be verified by ¹⁹F-NMR.
[Note 8]: Isopropanol was preferred as the use of less hindered alcohols (i.e., methanol) as eluents may promote decomposition of the product.
[Note 9]: Drying under high vacuum is often required to completely get rid of solvent residues. We consider this to be particularly important as these residues might be reactive under basic conditions (i.e., isopropanol) and promote undesired reaction pathways in the upcoming step.

Prepared by General procedure as above:

### Compound 2-TT (5-(octadec-1-en-1-yl)-5H-thianthren-5-ium tetrafluoroborate)

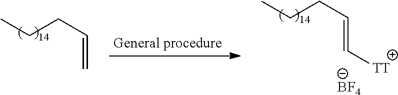

Following General procedure with the following modifications: carried out on a 4.5 mmol scale; 1 h reaction time at 25 °C Purification by flash column chromatography on flash silica gel (0% isopropanol in dichloromethane grading to 10% isopropanol in dichloromethane) afforded 1.51 g (61%, E:Z=93:7) of the title compound 2-TT.

Physical State: brown amorphous solid

¹H-NMR (499.64 MHz, DMSO-d⁶): δ=8.26 (dd, J=7.9, 1.4 Hz, 2H), 8.06 (dd, J=7.9, 1.3 Hz, 2H), 7.85 (td, J=7.7, 1.4 Hz, 2H), 7.77 (td, J=7.7, 1.3 Hz, 2H), 6.90-6.80 (m, 1H), 6.77 (d, J=14.8 Hz, 1H), 2.25 (q, J=7.0 Hz, 2H), 1.34 (q, J=7.1 Hz, 2H), 1.20 (d, J=29.8 Hz, 26H), 0.85 ppm (t, J=6.8 Hz, 3H).

¹³C-NMR (125.65 MHz, DMSO-d⁶): δ=154.3, 134.3, 134.1, 133.3, 129.9, 129.6, 121.0, 111.1, 32.0, 31.2, 28.97, 28.96 (2C), 28.95, 28.93 (2C), 28.9, 28.8, 28.6, 28.5, 28.2, 26.9, 22.0, 13.9 ppm.

¹⁹F-NMR (282.21 MHz, DMSO-d⁶): δ=−90.55 (s), —90.60 ppm (s)

HRMS (ESI): calcd. for C₃₀H₄₃S₂⁺ M⁺ 467.2801, found 467.2805.

TLC: Rf=0.60 (10% isopropanol in dichloromethane, CAM).

### Compound 22-TT (5-(hex-3-en-3-yl)-5H-thianthren-5-ium tetrafluoroborate)

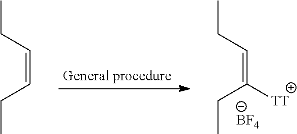

Following General procedure with the following modifications: starting from cis-hex-3-ene; 1 h reaction time at 25 °C Purification by flash column chromatography on flash silica gel (0% isopropanol in dichloromethane grading to 10% isopropanol in dichloromethane) afforded 820 mg (67%, E:Z=92:8) of the title compound 22-TT.

Physical State: brown solid.

¹H-NMR (499.64 MHz, DMSO-d⁶): S=8.42 (dd, J=8.0, 1.3 Hz, 2H), 8.07 (dd, J=8.0, 1.3 Hz, 2H), 7.91 (td, J=7.7, 1.4 Hz, 2H), 7.81 (td, J=7.7, 1.3 Hz, 2H), 5.51 (t, J=7.7 Hz, 1H), 2.28 (q, J=7.5 Hz, 2H), 2.16 (p, J=7.5 Hz, 2H), 0.87 (t, J=7.4 Hz, 3H), 0.72 ppm (t, J=7.5 Hz, 3H).

¹³C-NMR (125.65 MHz, DMSO-d⁶): S=142.9, 135.5, 135.2, 134.7, 129.9, 129.7, 124.2, 116.7, 22.1, 21.0, 12.8, 12.7 ppm.

¹⁹F-NMR (282.21 MHz, DMSO-d⁶): S=−90.55 (s), —90.60 ppm (s).

HRMS (ESI): calcd. for C₁₈H₁₉S₂⁺ M⁺ 299.0923, found 299.0924.

TLC: Rf=0.55 (10% isopropanol in dichloromethane, CAM).

### Compound 5-TT (5-(3-(3-phenylpropoxy)prop-1-en-1-yl)-5H-thianthren-5-ium tetrafluoroborate)

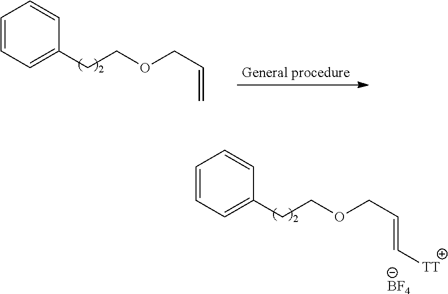

Following General procedure with the following modifications: 1 h reaction time at 25 °C Purification by flash column chromatography on flash silica gel (0% isopropanol in dichloromethane grading to 10% isopropanol in dichloromethane) afforded 1.01 g (67%, E:Z=34:66) of the title compound 5-TT.

Physical State: brown oil.

### E

¹H-NMR (499.64 MHz, DMSO-d⁶): S=8.30 (dd, J=7.9, 1.4 Hz, 2H), 8.07 (dd, J=8.0, 1.3 Hz, 2H), 7.87 (td, J=7.7, 1.4 Hz, 2H), 7.78 (td, J=7.7, 1.3 Hz, 2H), 7.32-7.29 (m, 2H), 7.25-7.19 (m, 3H), 6.74 (dt, J=14.7, 3.3 Hz, 1H), 6.69 (dt, J=14.6, 1.4 Hz, 1H), 4.11 (dd, J=3.4, 1.5 Hz, 2H), 3.36 (t, J=6.4 Hz, 2H), 2.54 (dd, J=8.6, 6.7 Hz, 2H), 1.80-1.70 ppm (m, 2H).

¹³C-NMR (125.65 MHz, DMSO-d⁶): S=148.9, 141.5, 134.7, 134.3, 134.2, 130.1, 129.6, 128.3, 128.2, 125.7, 120.0, 110.8, 69.7, 68.5, 31.6, 30.6 ppm

### Z

¹H-NMR (499.64 MHz, DMSO-d⁶): S=8.18 (dd, J=8.0, 1.3 Hz, 2H), 8.07 (d, J=1.3 Hz, 4H), 7.83 (td, J=7.7, 1.4 Hz, 3H), 7.74 (td, J=7.7, 1.3 Hz, 2H), 7.25-7.19 (m, 3H), 7.16-7.11 (m, 3H), 6.97 (dt, J=9.3, 3.9 Hz, 1H), 6.90 (dt, J=9.3, 2.0 Hz, 1H), 4.37 (dd, J=3.9, 2.0 Hz, 2H), 3.64 (t, J=6.6 Hz, 2H), 2.69 (dd, J=8.9, 6.7 Hz, 2H), 2.02-1.94 ppm (m, 2H).

¹³C-NMR (125.65 MHz, DMSO-d⁶): δ=149.0, 141.5, 134.5, 133.8, 133.1, 130.4, 129.4, 128.4 (2C), 125.9, 123.2, 113.8, 70.4, 68.0, 31.6, 30.5 ppm

¹⁹F-NMR (282.21 MHz, DMSO-d⁶): S=−90.55 (s), —90.60 ppm (s).

HRMS (ESI): calcd. for C₂₄H₁₃OS₂⁺ [M+H]⁺ 391.1185, found 391.1187.

TLC: Rf=0.50 (10% isopropanol in dichloromethane, CAM).

### Compound 6-TT (5-(7-(benzoyloxy)hept-1-en-1-yl)-5H-thianthren-5-ium tetrafluoroborate)

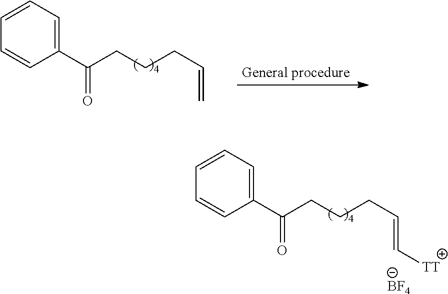

Following General procedure with the following modifications: carried out on a 1.25 mmol scale; 1 h reaction time at 25 °C Purification by flash column chromatography on flash silica gel (0% isopropanol in dichloromethane grading to 10% isopropanol in dichloromethane) afforded 554 mg (84%, E:Z=97:3) of the title compound 6-TT.

Physical State: colorless oil.

¹H-NMR (499.64 MHz, DMSO-d⁶): δ=8.25 (dd, J=7.9, 1.3 Hz, 2H), 8.04 (dd, J=7.9, 1.3 Hz, 2H), 7.94 (dd, J=8.4, 1.3 Hz, 2H), 7.84 (td, J=7.6, 1.4 Hz, 2H), 7.75 (td, J=7.7, 1.3 Hz, 2H), 7.66 (t, J=7.5 Hz, 1H), 7.53 (ddd, J=8.8, 7.4, 1.5 Hz, 2H), 6.91-6.76 (m, 2H), 4.21 (t, J=6.5 Hz, 2H), 2.30 (q, J=7.0 Hz, 2H), 1.66 (p, J=6.8 Hz, 2H), 1.44 (p, J=7.3 Hz, 2H), 1.34 ppm (p, J=7.0 Hz, 2H).

¹³C-NMR (125.65 MHz, DMSO-d⁶): δ=165.7, 154.2, 134.3, 134.1, 133.3, 133.2, 130.0, 129.8, 129.6, 129.0, 128.7, 121.0, 111.2, 64.4, 32.0, 27.7, 26.6, 24.8 ppm.

¹⁹F-NMR (282.21 MHz, DMSO-d⁶): δ=−90.55 (s), —90.60 ppm (s).

HRMS (ESI): calcd. for C₂₆H₂₅O₂S₂⁺ M⁺ 433.1290, found 433.1293.

TLC: Rf=0.45 (10% isopropanol in dichloromethane, CAM).

### Compound 7-TT (5-(5-cyanopent-1-en-1-yl)-5H-thianthren-5-ium tetrafluoroborate)

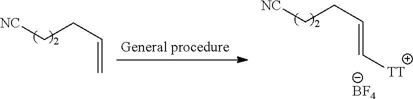

Following General procedure with the following modifications: 1.5 h reaction time at 25 °C Purification by flash column chromatography on flash silica gel (0% isopropanol in dichloromethane grading to 10% isopropanol in dichloromethane) afforded 525 mg (42%, E:Z=91:9) of the title compound 7-TT.

Physical State: brown wax.

¹H-NMR (499.64 MHz, DMSO-d⁶): δ=8.26 (dd, J=8.0, 1.4 Hz, 2H), 8.07 (dd, J=8.0, 1.3 Hz, 2H), 7.86 (td, J=7.7, 1.4 Hz, 2H), 7.77 (td, J=7.7, 1.3 Hz, 2H), 6.86-6.77 (m, 2H), 2.46 (t, J=7.2 Hz, 2H), 2.35 (td, J=7.4, 5.3 Hz, 2H), 1.69 ppm (p, J=7.3 Hz, 2H).

¹³C-NMR (125.65 MHz, DMSO-d⁶): δ=152.3, 134.4, 134.1, 133.4, 130.0, 129.6, 120.8, 120.1, 112.2, 31.0, 22.8, 15.5 ppm.

¹⁹F-NMR (282.21 MHz, DMSO-d⁶): δ=−90.55 (s), —90.60 ppm (s).

HRMS (ESI): calcd. for C₁₈H₁₆NS₂⁺ M⁺ 310.0719, found 310.0721.

TLC: Rf=0.50 (10% isopropanol in dichloromethane, CAM).

### Compound 8-TT (5-(6-(1,3-dioxoisoindolin-2-yl)hex-1-en-1-yl)-5H-thianthren-5-ium tetrafluoroborate)

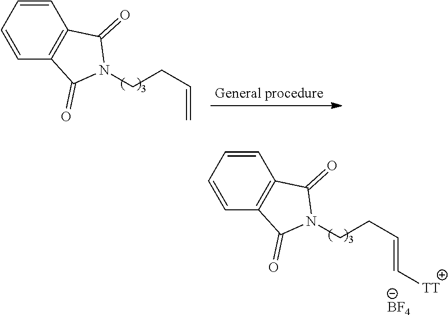

Following General procedure with the following modifications: carried out on a 10.0 mmol scale; 1 h reaction time at 25 °C Purification by flash column chromatography on flash silica gel (0% isopropanol in dichloromethane grading to 10% isopropanol in dichloromethane) afforded 3.10 g (58%, E:Z=97:3) of the title compound 8-TT.

Physical State: white solid.

¹H-NMR (499.64 MHz, DMSO-d⁶): δ=8.24 (d, J=7.9 Hz, 2H), 8.04 (d, J=7.9 Hz, 2H), 7.92-7.80 (m, 6H), 7.75 (t, J=7.7 Hz, 2H), 6.82-6.74 (m, 2H), 3.52 (t, J=6.9 Hz, 2H), 2.29 (q, J=6.7 Hz, 2H), 1.51 (p, J=7.3 Hz, 2H), 1.38 ppm (p, J=7.5 Hz, 2H).

¹³C-NMR (125.65 MHz, DMSO-d⁶): δ=167.9, 153.8, 134.43, 134.35, 134.1, 133.4, 131.6, 130.1, 129.6, 123.0, 121.0, 111.4, 36.9, 31.6, 27.3, 24.4 ppm.

¹⁹F-NMR (282.21 MHz, DMSO-d⁶): δ=−90.55 (s), —90.60 ppm (s).

HRMS (ESI): calcd. for C₂₆H₂₂NO₂S₂⁺ M⁺ 444.1068, found 444.1068.

TLC: Rf=0.50 (10% isopropanol in dichloromethane, CAM).

### Compound 12-TT (5-(octa-1,7-dien-1-yl)-5H-thianthren-5-ium tetrafluoroborate)

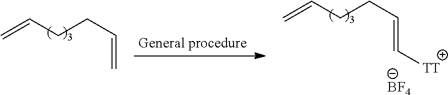

Following General procedure with the following modifications: 1 h reaction time at 25 °C Purification by flash column chromatography on flash silica gel (0% isopropanol in dichloromethane grading to 10% isopropanol in dichloromethane) afforded 760 mg (59%, E:Z=92:8) of the title compound 12-TT.

Physical State: brown oil.

¹H-NMR (499.64 MHz, DMSO-d⁶): δ=8.26 (dd, J=7.9, 1.4 Hz, 2H), 8.06 (dd, J=7.9, 1.4 Hz, 2H), 7.85 (td, J=7.7, 1.5 Hz, 2H), 7.77 (td, J=7.7, 1.3 Hz, 2H), 6.90-6.77 (m, 2H), 5.76-5.68 (m, 1H), 5.02-4.90 (m, 2H), 2.27 (q, J=6.9 Hz, 2H), 1.95 (q, J=7.0 Hz, 2H), 1.36 (p, J=7.3 Hz, 2H), 1.26 ppm (p, J=7.5 Hz, 2H).

¹³C-NMR (125.65 MHz, DMSO-d⁶): δ=154.2, 138.3, 134.3, 134.1, 133.4, 130.0, 129.6, 120.9, 114.8, 111.3, 32.5, 31.8, 27.3, 26.4 ppm

¹⁹F-NMR (282.21 MHz, DMSO-d⁶): δ=−90.55 (s), —90.60 ppm (s).

HRMS (ESI): calcd. for C₂₀H₂₁S₂⁺ M⁺ 325.1079, found 325.1083.

TLC: Rf=0.60 (10% isopropanol in dichloromethane, CAM).

### Compound 13-TT (5-(11-oxo-11-(prop-2-yn-1-yloxy)undec-1-en-1-yl)-5H-thianthren-5-ium tetrafluoroborate)

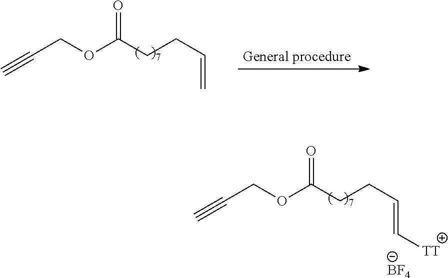

Following General procedure with the following modifications: 1.5 h reaction time at 25 °C Purification by flash column chromatography on flash silica gel (0% isopropanol in dichloromethane grading to 10% isopropanol in dichloromethane) afforded 1.28 g (78%, E:Z=96:4) of the title compound 13-TT.

Physical State: brown oil.

¹H-NMR (499.64 MHz, DMSO-d⁶): δ=8.26 (dd, J=8.0, 1.4 Hz, 2H), 8.06 (dd, J=7.9, 1.3 Hz, 2H), 7.85 (td, J=7.7, 1.4 Hz, 2H), 7.77 (td, J=7.7, 1.3 Hz, 2H), 6.87-6.76 (m, 2H), 4.67 (d, J=2.5 Hz, 2H), 3.52 (t, J=2.4 Hz, 1H), 2.31 (t, J=7.3 Hz, 2H), 2.25 (q, J=7.0 Hz, 2H), 1.49 (p, J=7.3 Hz, 2H), 1.35 (p, J=7.2 Hz, 2H), 1.23-1.14 ppm (m, 8H).

¹³C-NMR (125.65 MHz, DMSO-d⁶): δ=172.1, 154.3, 134.3, 134.1, 133.3, 129.9, 129.6, 120.9, 111.1, 78.5, 77.4, 51.4, 33.1, 32.0, 28.4, 28.3, 28.2, 28.1, 26.9, 24.2 ppm.

¹⁹F-NMR (282.21 MHz, DMSO-d⁶): δ=−90.55 (s), —90.60 ppm (s).

HRMS (ESI): calcd. for C₂₆H₂₉O₂S₂⁺ M⁺ 437.1603, found 437.1611.

TLC: Rf=0.55 (10% isopropanol in dichloromethane, CAM).

### Compound 15-TT (5-(6-chlorohex-1-en-1-yl)-5H-thianthren-5-ium tetrafluoroborate)

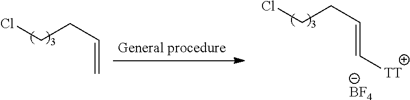

Following General procedure with the following modifications: 1 h reaction time at 25 °C Purification by flash column chromatography on flash silica gel (0% isopropanol in dichloromethane grading to 10% isopropanol in dichloromethane) afforded 1.02 g (77%, E:Z=94:6) of the title compound 15-TT.

Physical State: brown oil.

¹H-NMR (499.64 MHz, DMSO-d⁶): δ=8.27 (dd, J=8.0, 1.4 Hz, 2H), 8.07 (dd, J=8.0, 1.3 Hz, 2H), 7.86 (td, J=7.7, 1.4 Hz, 2H), 7.77 (td, J=7.7, 1.3 Hz, 2H), 6.84-6.77 (m, 2H), 3.58 (t, J=6.6 Hz, 2H), 2.29 (td, J=7.3, 5.4 Hz, 2H), 1.64 (p, J=6.9 Hz, 2H), 1.48 ppm (p, J=7.1 Hz, 2H).

¹³C-NMR (125.65 MHz, DMSO-d⁶): δ=153.6, 134.4, 134.1, 133.4, 130.0, 129.6, 120.8, 111.6, 44.8, 31.2, 31.1, 24.3 ppm.

¹⁹F-NMR (282.21 MHz, DMSO-d⁶): δ=−90.55 (s), —90.60 ppm (s).

HRMS (ESI): calcd. for C₁₈H₁₈ClS₂⁺ M⁺ 333.0533, found 333.0535.

TLC: Rf=0.50 (10% isopropanol in dichloromethane, CAM).

### Compound 17-TT (5-(10-(tosyloxy)dec-1-en-1-yl)-5H-thianthren-5-ium tetrafluoroborate)

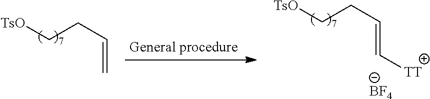

Following General procedure with the following modifications: carried out on a 4.5 mmol scale; 1 h reaction time at 25 °C Purification by flash column chromatography on flash silica gel (0% isopropanol in dichloromethane grading to 10% isopropanol in dichloromethane) afforded 1.40 g (51%, E:Z=98:2) of the title compound 17-TT.

Physical State: colorless oil.

¹H-NMR (499.64 MHz, DMSO-d⁶): δ=8.27 (dd, J=7.9, 1.3 Hz, 2H), 8.04 (dd, J=7.9, 1.2 Hz, 2H), 7.84 (dt, J=7.4, 1.0 Hz, 2H), 7.76 (dt, J=8.9, 3.6 Hz, 4H), 7.46 (d, J=8.0 Hz, 2H), 6.85 (dt, J=13.8, 6.8 Hz, 1H), 6.80-6.74 (m, 1H), 3.99 (t, J=6.3 Hz, 2H), 2.40 (s, 3H), 2.24 (q, J=7.2 Hz, 2H), 1.52 (p, J=6.6 Hz, 2H), 1.32 (t, J=7.1 Hz, 2H), 1.16 (t, J=7.2 Hz, 2H), 1.13-1.07 ppm (m, 6H).

¹³C-NMR (125.65 MHz, DMSO-d⁶): δ=154.3, 144.8, 134.3, 134.1, 133.3, 132.5, 130.1, 129.9, 129.6, 127.5, 120.9, 111.2, 70.8, 32.0, 28.2, 28.1, 28.0 (2C), 26.9, 24.6, 21.0 ppm.

¹⁹F-NMR (282.21 MHz, DMSO-d⁶): δ=−90.55 (s), —90.60 ppm (s).

HRMS (ESI): calcd. for C₂₉H₃₃O₃S₃⁺ M⁺ 525.1586, found 525.1588.

TLC: Rf=0.60 (10% isopropanol in dichloromethane, CAM).

### Example 9

### Preparation of Compound 3-TT vinyl (5-(2-phenylprop-1-en-1-yl)-5H-thianthren-5-ium tetrafluoroborate) and 3-TT allyl (5-(2-phenylallyl)-5H-thianthren-5-ium tetrafluoroborate) isomeric thianthren-5-ium salts

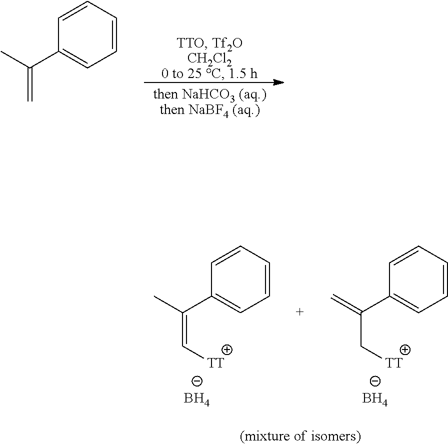

A solution of alkene (372 mg, 3.15 mmol, 1 equiv) and thianthrene S-oxide (TTO, 754 mg, 3.24 mmol, 1.03 equiv) in anhydrous dichloromethane (10 mL, 0.3 M) was cooled to 0 °C under argon atmosphere. [Note 1] At this temperature, trifluoromethane sulfonic anhydride (665 μL, 3.94 mmol, 1.25 equiv) was added dropwise. An immediate color change of the reaction mixture was observed upon addition and the blue solution was stirred at 0 °C for further 30 min. Then, the reaction mixture was warmed to 25 °C and stirred at this temperature for an additional 1 h until analysis by TLC indicated full conversion. At this point, saturated aqueous sodium bicarbonate solution (50 mL) was added, and the two-phase mixture was vigorously stirred at 25 °C for 15 min, whereupon a color change to brown was observed and the phases were separated. Then, aqueous sodium tetrafluoroborate solution (5 wt. %, 50 mL) was added to the organic phase and the mixture was stirred vigorously for 15 min and the phases were separated. This washing step using aqueous sodium tetrafluoroborate was repeated one more time. The layers were separated, and the organic phase was dried over sodium sulfate. The dried solution was filtered, and the filtrate was concentrated under reduced pressure. The residue was triturated with diethyl ether and dried under reduced pressure to yield the mixture of alkenyl and allylic thiantren-5-ium tetrafluoroborate salts 3-TT vinyl and 3-TT allyl (1.06 g, 80%, ratio 54:46). [Notes 2, 3]
[Note 1]: Dichloromethane was stabilized with amylene (not ethanol).
[Note 2]: Isopropanol readily reacts with the allylic isomer during column chromatography. [Note 3]: As both isomers are productive starting materials of the TT-Kornblum reaction, they were used as a mixture in the upcoming step.

Physical State: brown solid

### 3-TT Vinyl

¹H-NMR (499.64 MHz, CDCl₃): δ=8.45 (dd, J=7.9, 1.3 Hz, 2H), 7.85 (dd, J=7.9, 1.3 Hz, 2H), 7.71 (td, J=7.7, 1.4 Hz, 2H), 7.65 (td, J=7.7, 1.4 Hz, 2H), 7.41-7.36 (m, 1H), 7.34 (d, J=4.1 Hz, 4H), 6.76 (q, J=1.0 Hz, 1H), 2.78 ppm (d, J=1.1 Hz, 3H).

¹³C-NMR (125.65 MHz, CDCl₃): δ=161.4, 137.7, 135.6, 134.2, 133.7, 131.1, 130.3, 130.3, 129.0, 126.5, 121.6, 105.8, 19.4 ppm

### 3-TT Allyl

¹H-NMR (499.64 MHz, CDCl₃): δ=7.95 (dd, J=7.9, 1.3 Hz, 2H), 7.71 (ddd, J=7.8, 4.4, 1.7 Hz, 2H), 7.68-7.63 (m, 2H), 7.53 (td, J=7.7, 1.4 Hz, 2H), 7.31 (td, J=6.8, 3.1 Hz, 3H), 7.27 (dd, J=6.9, 2.5 Hz, 2H), 5.58 (d, J=11.6 Hz, 2H), 5.00 ppm (s, 2H).

¹³C-NMR (125.65 MHz, CDCl₃): δ=135.9, 135.0, 134.4, 129.7, 129.6, 129.2, 129.0, 128.7, 127.7, 126.2, 124.7, 116.6, 45.4 ppm

¹⁹F-NMR (282.21 MHz, DMSO-d⁶): 6=−90.55 (s), —90.60 ppm (s).

HRMS (ESI): calcd. for C₂₁H₁₇S₂⁺ [M+H]⁺ 333.0766, found 333.0770.

### Example 10

### Preparation of 9-TT (5-(10-carboxydec-1-en-1-yl)-5H-thianthren-5-ium tetrafluoroborate)

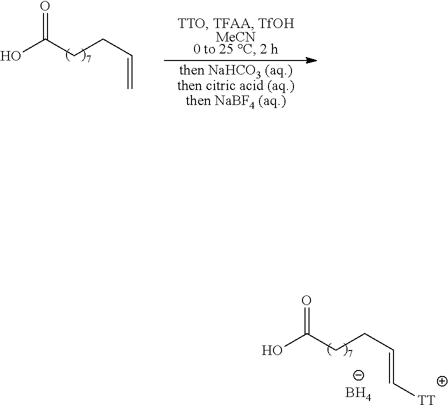

A suspension of alkene (580 mg, 3.15 mmol, 1 equiv) and thianthrene S-oxide (TTO, 754 mg, 3.24 mmol, 1.03 equiv) in anhydrous acetonitrile (10 mL, 0.3 M) was cooled to 0 °C under argon atmosphere. At this temperature, trifluoroacetic anhydride (TFAA, 1.33 mL, 9.45 mmol, 3.00 equiv) and trifluoromethanesulfonic acid (560 μL, 6.30 mmol, 2.00 equiv) were added dropwise consecutively. A delayed color change of the reaction mixture was observed upon addition and the blue solution was stirred at 0 °C for a further 1 h. [Note 1] Then, the reaction mixture was warmed to 25 °C and stirred at this temperature for an additional 1 h until analysis by TLC indicated full conversion. At this point, the reaction mixture was concentrated under reduced pressure [Note 2] and subsequently dissolved in dichloromethane (30 mL). Saturated aqueous sodium bicarbonate solution (50 mL) was added, and the two-phase mixture was vigorously stirred at 25 °C for 15 min, whereupon a color change to brown was observed. Aqueous citric acid solution (10 wt. %, approx. 50 mL) was added until pH=6 and the phases were separated. [Note 3] Then, aqueous sodium tetrafluoroborate solution (5 wt. %, 50 mL) was added to the organic phase and the mixture was stirred vigorously for 15 min and the phases were separated. This washing step was repeated one more time. The layers were separated, and the organic phase was dried over sodium sulfate. The dried solution was filtered, and the filtrate was concentrated under reduced pressure. The residue was triturated with diethyl ether and dried under reduced pressure to yield compound 9-TT alkenyl thiantren-5-ium tetrafluoroborate salt (1.24 g, 81%) [Note 4]
[Note 1]: In the presence of functional groups which are likely to be acetylated by trifluoroacetic anhydride (i.e., carboxylic acids), the color change is not immediate but occurs during addition.
[Note 2] Water jet vacuum pumps were used with gentle heating by a 30 °C water bath.
[Note 3] This step is required to ensure the protonation of the carboxylic acid.
[Note 4] No column chromatography was performed due to the particularly high polarity and acidity of the product.

Physical State: brown oil

¹H-NMR (499.64 MHz, DMSO-d⁶): δ=11.99 (br s, 1H), 8.26 (dd, J=8.0, 1.4 Hz, 2H), 8.06 (dd, J=7.9, 1.4 Hz, 2H), 7.85 (td, J=7.7, 1.4 Hz, 2H), 7.77 (td, J=7.7, 1.3 Hz, 2H), 6.91-6.75 (m, 2H), 2.51-2.46 (m, 1H), 2.25 (q, J=7.0 Hz, 2H), 2.17 (t, J=7.4 Hz, 1H), 1.48 (dt, J=24.7, 7.2 Hz, 2H), 1.35 (t, J=7.1 Hz, 2H), 1.26-1.13 ppm (m, 8H).

¹³C-NMR (125.65 MHz, DMSO-d⁶): δ=174.5, 154.4, 134.4, 134.1, 133.4, 130.0, 129.6, 121.0, 111.2, 33.6, 32.1, 28.6, 28.5, 28.4, 28.2, 27.0, 24.5 ppm.

¹⁹F-NMR (282.21 MHz, DMSO-d⁶): δ=−90.55 (s), —90.60 ppm (s).

HRMS (ESI): calcd. for C₂₃H₂₇O₂S₂⁺ M⁺ 399.1447, found 399.1448.

TLC: Rf=0.35 (10% isopropanol in dichloromethane, CAM).

### Example 11

### Preparation of 1-TT (5-(but-1-en-1-yl)-5H-thianthren-5-ium tetrafluoroborate)

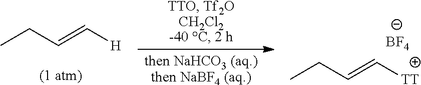

A round-bottom flask (100 mL) capped with a rubber septum and equipped with a stirring bar was charged with the solution of thianthrene S-oxide (TTO, 1.00 g, 4.30 mmol, 1 equiv) in anhydrous dichloromethane (35.0 mL, 0.125 M) and the mixture was cooled to −40 °C (internal temperature). Then, 1-butene gas was bubbled through the solution for 15 minutes, after which balloons filled with 1-butene were connected to the flask to maintain the 1-butene atmosphere throughout the reaction. Trifluoromethane sulfonic anhydride (873 μL, 5.17 mmol, 1.20 equiv) was added dropwise to the reaction, whereupon a dark purple suspension was progressively formed. After stirring at this temperature for further 30 min, the cooling bath was removed, and the mixture was stirred at 25 °C for 1.5 h until analysis by LC-MS indicated full conversion. At this point, the balloons and the rubber septum were removed, and saturated aqueous sodium bicarbonate solution (35 mL) was added carefully. The two-phase system was vigorously shaken in a separation funnel, phases were separated, and the aqueous layer was extracted with dichloromethane (2×20 mL). All organic phases were then combined and partially concentrated (to around 25 mL). Then, this organic phase was thoroughly washed with aqueous sodium tetrafluoroborate solution (5 wt. %, 5×30 mL) and dried over sodium sulfate. [Note 1] The dried solution was filtered, and the filtrate was concentrated under reduced pressure. The residue was purified by flash column chromatography on silica gel (5% isopropanol in dichloromethane grading to 10% isopropanol in dichloromethane) to yield alkenyl 1-TT thiantren-5-ium tetrafluoroborate salt (1.04 g, 2.90 mmol, 68%, E:Z=92:8) as a brown oil. [Notes 2,3]
[Note 1]: This step promotes counterion exchange; completeness may be practically verified by ¹⁹F-NMR.
[Note 2]: Isopropanol was preferred as the use of less hindered alcohols (i.e., methanol) as eluents may promote decomposition of the product.
[Note 3]: Drying under high vacuum is often required to completely get rid of solvent residues. This is also important as these residues might be reactive under basic conditions (i.e., isopropanol) and promote undesired reaction pathways in the upcoming step.

Physical State: brown oil

¹H-NMR (499.64 MHz, DMSO-d⁶): δ=8.26 (dd, J=8.0, 1.4 Hz, 2H), 8.06 (dd, J=7.9, 1.3 Hz, 2H), 7.85 (td, J=7.7, 1.4 Hz, 2H), 7.77 (td, J=7.7, 1.3 Hz, 2H), 6.97-6.86 (m, 1H), 6.76 (dt, J=14.6, 1.6 Hz, 1H), 2.29 (tt, J=7.4, 5.9 Hz, 2H), 0.97 ppm (t, J=7.4 Hz, 3H).

¹³C-NMR (125.65 MHz, DMSO-d⁶): δ=156.0, 134.3, 134.1, 133.3, 130.0, 129.6, 121.0, 110.3, 25.7, 11.6 ppm.

¹⁹F-NMR (282.21 MHz, DMSO-d⁶): δ=−90.55 (s), —90.60 ppm (s)

HRMS (ESI): calcd. for C₂₉H₃₃O₃S₃⁺ M⁺ 271.0610, found 271.0611.

TLC: Rf=0.45 (10% isopropanol in dichloromethane, CAM).

### Example 12

### Compound 10-TT (5-(10-hydroxydec-1-en-1-yl)-5H-thianthren-5-ium tetrafluoroborate)

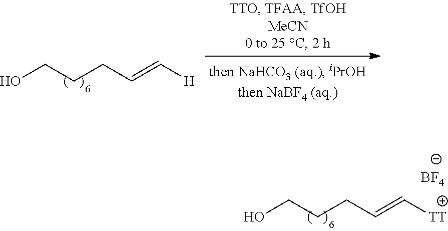

A suspension of alkene (492 mg, 3.15 mmol, 1 equiv) and thianthrene S-oxide (TTO, 754 mg, 3.24 mmol, 1.03 equiv) in anhydrous acetonitrile (10 mL, 0.3 M) was cooled to 0 °C under argon atmosphere. [Note 1] At this temperature, trifluoroacetic anhydride (1.78 mL, 12.6 mmol, 4.00 equiv) and trifluoromethanesulfonic acid (560 μL, 6.30 mmol, 2.00 equiv) were added dropwise consecutively. A delayed color change of the reaction mixture was observed upon addition and the lilac/blue solution was stirred at 0 °C for a further 1 h. [Note 2] Then, the reaction mixture was warmed to 25 °C and stirred at this temperature for an additional 1 h until analysis by TLC indicated full conversion. [Note 3] At this point, the reaction mixture was concentrated under reduced pressure [Note 4] and subsequently dissolved in isopropanol (20 mL). Saturated aqueous sodium bicarbonate solution (40 mL) was added, and the two-phase mixture was vigorously stirred at 25 °C for 150 min until analysis by TLC indicated full conversion. [Notes 5,6] Then, dichloromethane (40 mL) was added and the phases were separated. Aqueous sodium tetrafluoroborate solution (5 wt. %, 50 mL) was added to the organic phase and the mixture was stirred vigorously for 15 min and the phases were separated. This washing step was repeated one more time. [Note 7] The layers were separated, and the organic phase was dried over sodium sulfate. The dried solution was filtered, and the filtrate was concentrated under reduced pressure. The residue was purified by flash column chromatography on silica gel (0-10% isopropanol in dichloromethane) to yield compound 10-TT alkenyl thiantren-5-ium tetrafluoroborate salt (1.05 g, 2.29 mmol, 73%) as a brown oil. [Notes 8,9]
[Note 1]: An ice bath was used.
[Note 2]: In the presence of functional groups which are likely to be acetylated by trifluoroacetic anhydride (i.e., alcohols), the color change is not immediate but occurs during addition.
[Note 3]: In this specific case, two thianthren-5-ium salts appeared on TLC: one with a trifluoroacetylated hydroxyl group and one with a free hydroxyl group.
[Note 4]: Water jet vacuum pumps were used with gentle heating by a 30 °C water bath.
[Note 5]: This step promotes the formation of alkenyl thianthren-5-ium salts from the precursor dicationic species and the hydrolyzation of the trifluoroacetyl esters.
[Note 6]: Full conversion leaves only one spot on the TLC out of the two characteristic spots of alkenyl thianthren-5-ium salts.
[Note 7]: This step completes the counterion exchange.
[Note 8]: Isopropanol was preferred as the use of less hindered alcohols (i.e., methanol) as eluents may promote decomposition of the product.
[Note 9]: Drying under high vacuum is often required to completely get rid of solvent residues. This is also important as these residues might be reactive under basic conditions (i.e., isopropanol) and promote undesired reaction pathways in the upcoming step.

Physical State: brown oil

TLC: Rf=0.55 (10% isopropanol in dichloromethane, CAM).

Spectral properties were in accordance with those reported in the literature. (ref xxii)

### Example 13

### Compound 11-TT (5-(10-oxodec-1-en-1-yl)-5H-thianthren-5-ium tetrafluoroborate)

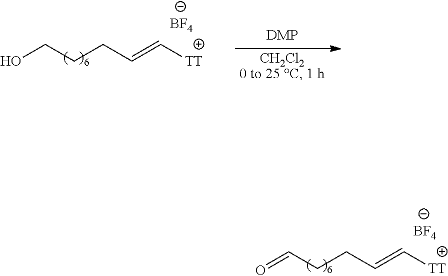

AA solution of alcohol (756 mg, 1.65 mmol, 1 equiv) in dichloromethane (8.0 mL, 0.2 M) was cooled to 0 °C [Note 1] To this solution was added Dess-Martin periodinane (DMP, 805 mg, 1.90 mmol, 1.15 equiv) in one portion. The mixture was allowed to warm to 25 °C and stirred at this temperature for 1 h when analysis by TLC indicated full conversion. At this point, saturated aqueous sodium bicarbonate solution (25 mL) and dichloromethane (20 mL) were added, and the phases were separated. Then, the organic layer was washed with aqueous sodium tetrafluoroborate solution (5 wt. %, 50 mL) and dried over sodium sulfate. [Note 2] The dried inhomogeneous solution was filtered through a plug of Celite and the clear filtrate was concentrated under reduced pressure. [Note 3] The residue was purified by flash column chromatography on silica gel (0-10% isopropanol in dichloromethane) to yield compound 11-TT aldehyde (545 mg, 1.19 mmol, 72%, E:Z 96:4) as a yellow amorphous solid.
[Note 1] Dichloromethane was stabilized with amylene (not ethanol).
[Note 2] This step ensures persistent counterion composition.
[Note 3] A white-colored byproduct precipitate derived from the DMP reagent was removed by the filtration.

Physical State: yellow amorphous solid

¹H-NMR (499.64 MHz, DMSO-d⁶): δ=9.65 (t, J=1.7 Hz, 1H), 8.26 (dd, J=7.9, 1.4 Hz, 2H), 8.06 (dd, J=7.9, 1.2 Hz, 2H), 7.85 (td, J=7.7, 1.4 Hz, 2H), 7.77 (td, J=7.7, 1.3 Hz, 2H), 6.89-6.72 (m, 2H), 2.39 (td, J=7.2, 1.7 Hz, 2H), 2.26 (q, J=7.0 Hz, 2H), 1.46 (p, J=7.2 Hz, 2H), 1.35 (p, J=7.2 Hz, 2H), 1.19 ppm (q, J=3.8 Hz, 6H).

¹³C-NMR (125.65 MHz, DMSO-d⁶): δ=203.4, 154.3, 134.3, 134.1, 133.3, 129.9, 129.6, 120.9, 111.2, 42.9, 32.0, 28.3, 28.2, 27.9, 26.8, 21.3 ppm.

¹⁹F-NMR (282.21 MHz, DMSO-d⁶): δ=−90.55 (s), —90.60 ppm (s)

HRMS (ESI): calcd. for C₂₂H₂₅OS₂⁺ M⁺ 369.1341, found 369.1344.

TLC: Rf=0.40 (10% isopropanol in dichloromethane, CAM).

### Summarizing the Synthesis of Conjugated Diene Pheromones, Kairomones, and Related Compounds (Examples 14-XX)

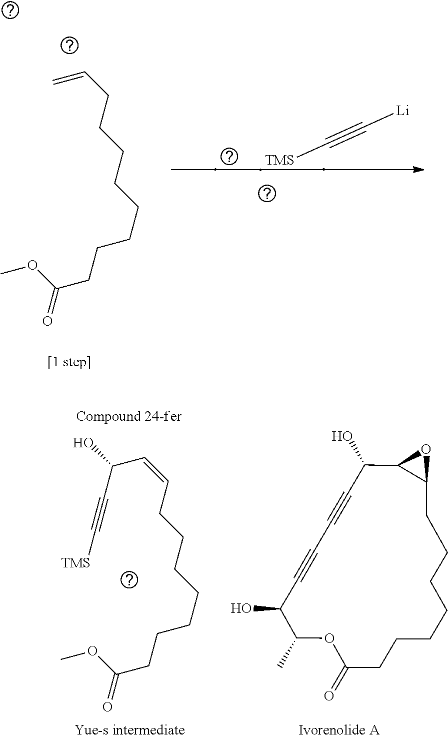

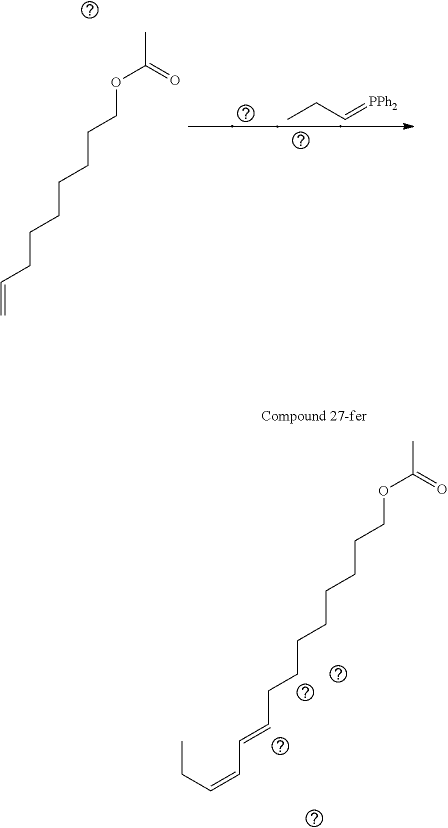

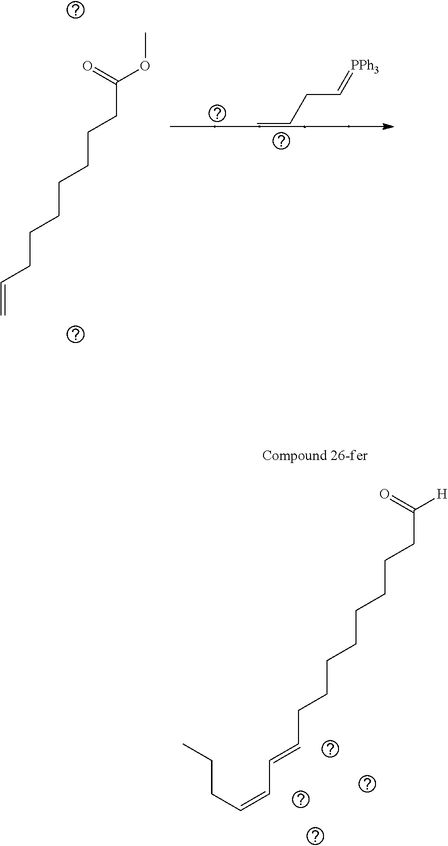

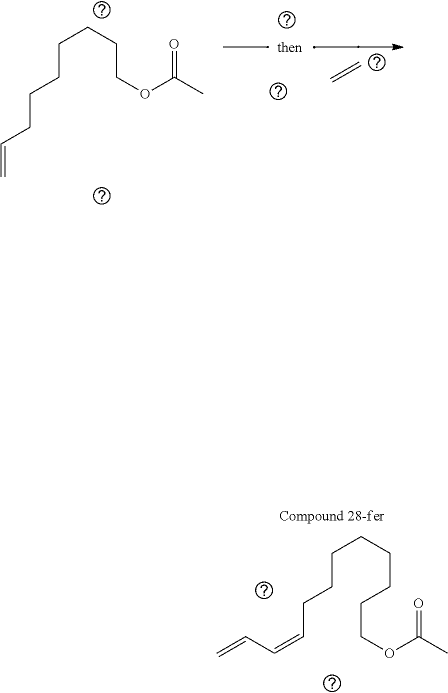

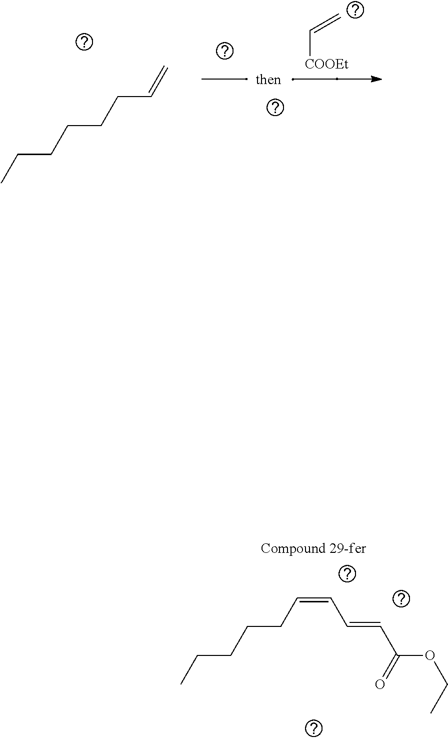

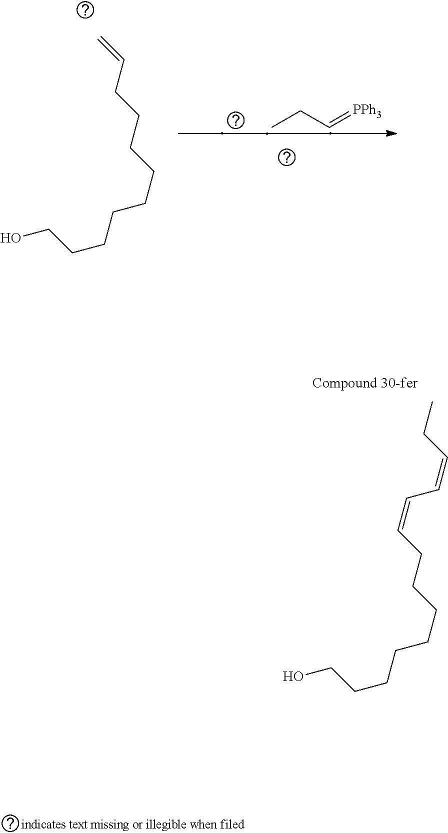

### Example 14

### Synthesis of Yue's intermediate towards Ivorenolide A

Upscaled, slightly modified versions of our general procedures for the preparation of alkenyl thianthren-5-ium salts and unsaturated carbonyls, and Yue's protocol were used as follows:

### Preparation of Alcohol 24-fer (methyl (Z)-11-hydroxy-13-(trimethylsilyl)tridec-9-en-12-ynoate)

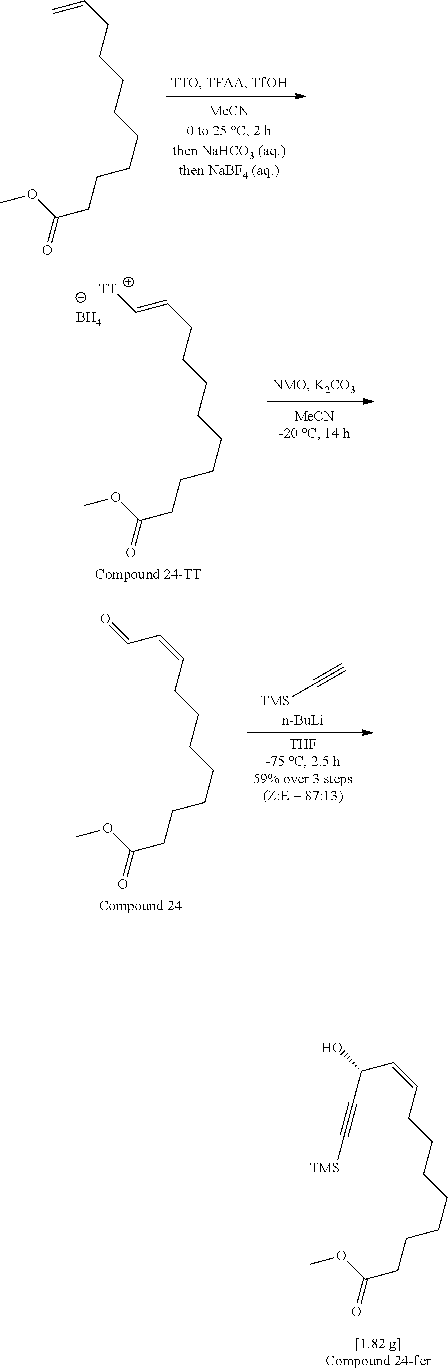

A suspension of methyl undec-10-enoate (1.98 g, 10.0 mmol, 1 equiv) and thianthrene S-oxide (TTO, 2.39 g, 10.3 mmol, 1.03 equiv) in anhydrous acetonitrile (30 mL, 0.3 M) was cooled to 0 °C (internal temperature) under argon atmosphere. Then, trifluoroacetic anhydride (TFAA, 4.24 mL, 40.0 mmol, 3.00 equiv) and trifluoromethanesulfonic acid (1.78 mL, 30.0 mmol, 2.00 equiv) were added consecutively dropwise, while maintaining the internal temperature below 10 °C [Note 1] An immediate color change of the reaction mixture was observed upon the first addition step and the lilac solution was stirred at 0 °C for a further 1 h. Then, the reaction mixture was warmed to 25 °C and stirred at this temperature for an additional 1 h, when analysis by TLC indicated full conversion. At this point, the reaction mixture was concentrated under reduced pressure and subsequently dissolved in dichloromethane (100 mL). [Note 2] Saturated aqueous sodium bicarbonate solution (100 mL) was added, and the two-phase mixture was vigorously stirred at 25 °C for 15 min, whereupon a color change to brown was observed. [Note 3] The phases were separated and aqueous sodium tetrafluoroborate solution (5 wt. %, 150 mL) was added to the organic phase and the mixture was stirred vigorously for 10 min and the phases were separated. This washing step was repeated two more times. [Note 4] The layers were separated, and the organic phase was dried over sodium sulfate. The dried solution was filtered, and the filtrate was concentrated under reduced pressure. The crude alkenyl 24-TT thianthren-5-ium tetrafluoroborate was used in the next step without further purification.

### Compound 24-TT

¹H NMR (500 MHz, DMSO) δ=8.26 (dd, J=7.9, 1.4 Hz, 2H), 8.06 (dd, J=7.9, 1.3 Hz, 2H), 7.85 (td, J=7.7, 1.4 Hz, 2H), 7.77 (td, J=7.7, 1.3 Hz, 2H), 6.91-6.74 (m, 2H), 3.58 (s, 3H), 2.32-2.20 (m, 4H), 1.49 (h, J=7.3 Hz, 2H), 1.34 (q, J=7.0 Hz, 2H), 1.23-1.12 (m, 8H).
[Note 1]: Caution! Special attention should be taken when adding the corrosive trifluoromethanesulfonic acid dropwise at this scale as disposable syringes/needles may disintegrate.
[Note 2]: Water jet vacuum pumps were used with gentle heating by a 30 °C water bath.
[Note 3]: This step promotes the formation of alkenyl thianthrenium salts from the precursor dicationic species.
[Note 4]: This step completes the counterion exchange. Completeness may be practically verified by ¹⁹F-NMR.

A suspension of N-methylmorpholine N-oxide (NMO, 4.69 g, 40 mmol, 4.00 equiv) and well powdered, oven-dried potassium carbonate (6.91 g, 50 mmol, 5.00 equiv) in anhydrous acetonitrile (75 mL) was cooled to −20 °C (±2 °C, internal temperature) and stirred at this temperature for 30 min. [Notes 5,6] Then, a solution of the crude alkenyl thianthren-5-ium tetrafluoroborate in anhydrous acetonitrile (25 mL) was added dropwise while maintaining the internal temperature below −18 °C The reaction mixture was stirred at this temperature for 14 h when analysis by TLC indicated full conversion. Then, saturated aqueous sodium bicarbonate solution (400 mL) and dichloromethane (400 mL) were added subsequently. The layers were separated, and the organic phase was washed with saturated aqueous sodium bicarbonate solution (2×200 mL) and dried over sodium sulfate. [Note 7] The dried solution was filtered, and the filtrate was concentrated under reduced pressure. [Note 8] The crude 24 enal was used in the next step without further purification.

### Compound 24

¹H NMR (500 MHz, CDCl₃) δ=10.08 (d, J=8.1 Hz, 1H), 6.64 (dd, J=11.2, 8.2 Hz, 1H), 6.00-5.91 (m, 1H), 3.66 (s, 3H), 2.64-2.57 (m, 2H), 2.27 (d, J=7.4 Hz, 2H), 1.64-1.57 (m, 2H), 1.53-1.46 (m, 2H), 1.38-1.32 ppm (m, 6H).
[Note 5]: A low-temperature thermostat was used to control cooling bath temperature.
[Note 6]: This preliminary stirring ensures that the internal temperature stabilizes as initial overcooling to ca. −24 °C may occur due to the endothermic dissolution of K₂CO₃.
[Note 7]: These steps eliminate excess NMO and N-alkylated by-products.
[Note 8]: To avoid isomerization of the (Z)-enal, the water bath temperature was set to 30 °C

A solution of ethynyltrimethylsilane (1.1 mL, 7.5 mmol, 0.75 equiv) in anhydrous tetrahydrofuran (46.7 mL) was cooled to −75 °C (±2 °C, internal temperature). Then, n-BuLi in hexanes (3.0 mL, 2.5 M, 7.5 mmol, 0.75 equiv) was added dropwise, while maintaining the internal temperature below −70 °C and the mixture was stirred for an additional 20 min. Then, a solution of the crude enal in anhydrous tetrahydrofuran (20 mL) was added dropwise while maintaining the internal temperature below −70 °C and the mixture was stirred for an additional 2.5 h. At this point, saturated aqueous ammonium chloride solution (60 mL) was added, and under stirring the two-phase system was allowed to warm to 25 °C Then, ethyl acetate (150 mL) was added, and the phases were separated. The aqueous phase was extracted with ethyl acetate (2×100 mL). The combined organic phases were washed with brine (150 mL) and dried over sodium sulfate. The dried solution was filtered, and the filtrate was concentrated under reduced pressure. The residue was purified by flash column chromatography on silica gel (0-20% ethyl acetate in hexanes) to afford 24-fer alcohol (1.82 g, 5.86 mmol, 59%, Z:E=87:13) as a colorless oil.

### Compound 24-fer

¹H-NMR (499.64 MHz, CDCl₃): δ=5.55-5.50 (m, 2H), 5.10 (dd, J=6.8, 4.4 Hz, 1H), 3.64 (s, 3H), 2.28 (t, J=7.6 Hz, 2H), 2.17-2.03 (m, 3H), 1.59 (t, J=7.4 Hz, 2H), 1.36 (t, J=6.7 Hz, 2H), 1.28 (d, J=4.1 Hz, 5H), 0.21-0.07 ppm (m, 9H).

¹³C-NMR (125.65 MHz, CDCl₃): δ=174.3, 133.5, 129.0, 105.6, 89.3, 58.5, 51.4, 34.0, 29.1, 29.0 (2C), 28.9, 27.5, 24.8, −0.2 ppm.

TLC: Rf=0.30 (10% ethyl acetate in hexanes, CAM).

### Example 15

### Synthesis of the Sex Pheromone of the Horse-Chestnut Leaf Miner

Upscaled, modified versions of our general procedures for the preparation of alkenyl thianthren-5-ium salts and unsaturated carbonyls were used as follows: (1) Tf₂O instead of TFAA and TfOH was used to demonstrate the feasibility of this alternative process; (2) No counterion exchange was performed as triflates are also appropriate starting materials of the TT-Kornblum reaction; (3) NaHCO₃ instead of K₂CO₃ and elevated temperature were used as more suitable conditions to promote the formation of the desired (E)-enal.

### Preparation of ester 25-fer (methyl (8E,10Z)-tetradeca-8,10-dienoate)

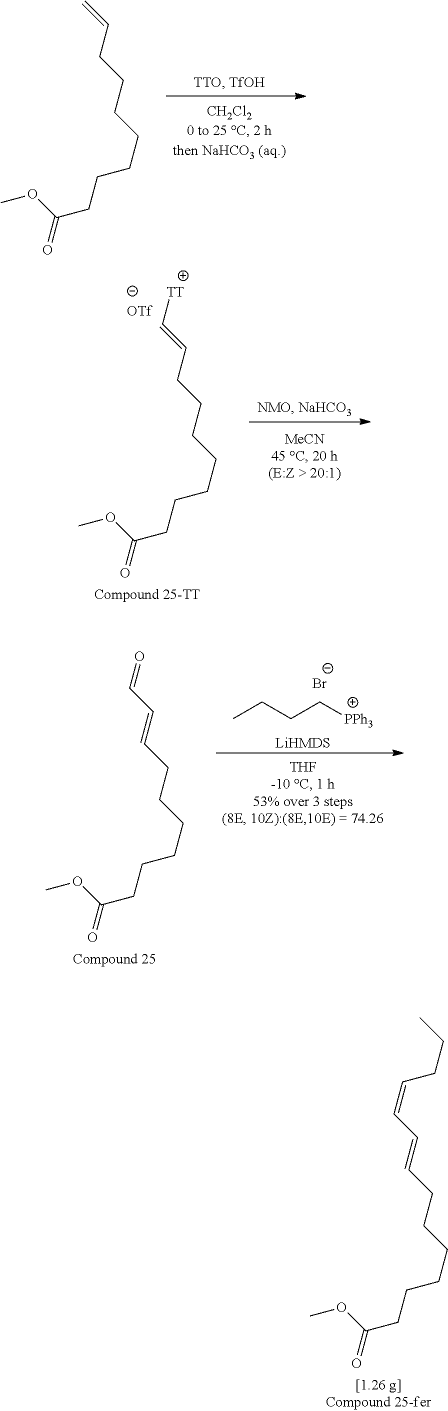

A solution of methyl 9-decenoate (1.84 g, 10.0 mmol, 1 equiv) and thianthrene S-oxide (TTO, 2.56 g, 11.0 mmol, 1.10 equiv) in anhydrous dichloromethane (100 mL, 0.1 M) was cooled to 0 °C (internal temperature) under argon atmosphere. Then, trifluoromethanesulfonic anhydride (2.11 mL, 12.5 mmol, 1.25 equiv) was added dropwise, while maintaining the internal temperature below 10 °C An immediate color change of the reaction mixture was observed upon addition and the lilac solution was stirred at 0 °C for a further 1 h. Then, the reaction mixture was warmed to 25 °C and stirred at this temperature for an additional 1 h, when a color change to brown was observed and analysis by TLC indicated full conversion. At this point, saturated aqueous sodium bicarbonate solution (100 mL) was added, and the two-phase mixture was vigorously stirred at 25 °C for 15 min. [Note 1] The phases were separated, and the organic phase was dried over sodium sulfate. The dried solution was filtered, and the filtrate was concentrated under reduced pressure to yield the crude 25-TT thianthren-5-ium salt.

### Compound 25-TT

¹H NMR (500 MHz, DMSO) δ=8.26 (dd, J=8.0, 1.4 Hz, 2H), 8.06 (dd, J=7.9, 1.3 Hz, 2H), 7.85 (td, J=7.7, 1.4 Hz, 2H), 7.77 (td, J=7.7, 1.4 Hz, 2H), 6.91-6.74 (m, 2H), 3.58 (s, 3H), 3.36 (d, J=3.6 Hz, 2H), 2.26 (tq, J=7.0, 3.5 Hz, 4H), 1.46 (p, J=7.3 Hz, 2H), 1.35 (p, J=7.1 Hz, 2H), 1.21-1.14 (m, 6H).
[Note 1]: This step promotes the formation of alkenyl thianthrenium salts from the precursor dicationic species.

A solution of the crude alkenyl thianthren-5-ium salt in acetonitrile (100 mL, 0.1 M) was stirred under argon atmosphere at 45 °C To this solution was added N-methylmorpholine N-oxide monohydrate (NMO, 5.41 g, 40 mmol, 4.00 equiv) and well-powdered sodium bicarbonate (4.20 g, 50 mmol, 5.00 equiv) in one portion, and the suspension was stirred at this temperature for 20 h. [Note 2] Then, saturated aqueous sodium bicarbonate solution (200 mL) and diethyl ether (400 mL) were added subsequently. The layers were separated, and the organic phase was washed with saturated aqueous sodium bicarbonate solution (2×200 mL) and brine (200 mL), and was dried over sodium sulfate. The dried solution was filtered, and the filtrate was concentrated under reduced pressure.
[Note 3] The crude 25 enal was used in the next step without further purification.

### Compound 25

¹H NMR (500 MHz, CDCl₃) δ=9.43 (dd, J=7.9, 1.2 Hz, 1H), 6.80 (dt, J=15.5, 6.8 Hz, 1H), 6.05-6.00 (m, 1H), 3.59 (s, 3H), 2.29-2.20 (m, 4H), 1.64-1.57 (m, 2H), 1.53-1.46 (m, 2H), 1.38-1.32 ppm (m, 4H).
[Note 2]: Technical grade acetonitrile and NMO monohydrate were used as the reaction is not sensitive to water.
[Note 3]: Water bath temperature was set to 30 °C due to the possible volatility of the product.

A suspension of butyltriphenylphosphonium bromide (3.99 g, 10.0 mmol, 1.00 equiv) in anhydrous tetrahydrofuran (75 mL) was cooled to −15 °C (±2 °C, internal temperature). Then, lithium bis(trimethylsilyl)amide in tetrahydrofuran (LiHMDS, 10.0 mL, 1.0 M, 10.0 mmol, 1.00 equiv) was added dropwise, while maintaining the internal temperature below −10 °C The bright orange mixture was warmed to 0 °C and stirred at this temperature for an additional 40 min. After cooling back to −15 °C, a solution of the crude enal in anhydrous tetrahydrofuran (25 mL) was added dropwise while maintaining the internal temperature below −10 °C and the mixture was stirred at this temperature for an additional 60 min. At this point, analysis by TLC indicated full conversion, and saturated aqueous sodium bicarbonate solution (200 mL) was added. The well-stirred two-phase system was allowed to warm to 25 °C Then, diethyl ether (400 mL) was added, and the phases were separated. The organic phase was washed with saturated aqueous sodium bicarbonate solution (2×200 mL) and brine (200 mL), and was dried over sodium sulfate. The dried solution was filtered, and the filtrate was concentrated under reduced pressure. The residue was redissolved in hexanes (100 mL), filtered and the precipitate was washed with hexanes. [Note 4] The filtrate was concentrated under reduced pressure and the residue was purified by flash column chromatography on silica gel (0-20% ethyl acetate in hexanes) to afford 25-fer diene (1.26 g, 5.29 mmol, 53%, Z:E=74:26) as a colorless oil.
[Note 4]: The washing was continued until the washing solution did not contain the desired product as indicated by TLC analysis.

### Compound 25-fer

### E,Z

¹H-NMR (499.64 MHz, CDCl₃): δ=6.31-6.23 (m, 1H), 6.03-5.90 (m, 1H), 5.63 (dt, J=14.6, 7.0 Hz, 1H), 5.30 (dt, J=10.9, 7.6 Hz, 1H), 3.66 (s, 3H), 2.29 (td, J=7.5, 2.2 Hz, 2H), 2.16-2.11 (m, 1H), 2.08 (q, J=7.5 Hz, 1H), 2.03 (t, J=7.1 Hz, 1H), 1.63-1.59 (m, 2H), 1.42-1.39 (m, 3H), 1.34-1.29 (m, 5H), 0.90 ppm (t, J=7.4 Hz, 3H).

¹³C-NMR (125.65 MHz, CDCl₃): δ=174.3, 134.4, 129.9, 128.7, 125.7, 51.4, 34.0, 32.7, 29.7, 29.1, 29.0, 28.8, 24.9, 22.9, 13.8 ppm.

### E,E

¹H-NMR (499.64 MHz, CDCl₃): δ=6.03-5.90 (m, 2H), 5.58-5.51 (m, 2H), 3.66 (s, 3H), 2.29 (td, J=7.5, 2.2 Hz, 2H), 2.16-2.11 (m, 1H), 2.08 (q, J=7.5 Hz, 1H), 2.03 (t, J=7.1 Hz, 1H), 1.63-1.59 (m, 2H), 1.42-1.39 (m, 3H), 1.34-1.29 (m, 5H), 0.89 ppm (t, J=7.2 Hz, 3H).

¹³C-NMR (125.65 MHz, CDCl₃): δ=174.3, 132.3, 132.1, 130.42, 130.39, 51.4, 34.7, 32.5, 29.7, 29.2, 29.0, 28.8, 24.9, 22.5, 13.7 ppm.

HRMS (EI): calcd. for C₁₅H₂₆O₂⁺ M⁺ 238.1927, found 238.1924.

TLC: Rf=0.45 (10% ethyl acetate in hexanes, CAM).

### Preparation of aldehyde 26-fer ((8E,10Z)-tetradeca-8,10-dienal)

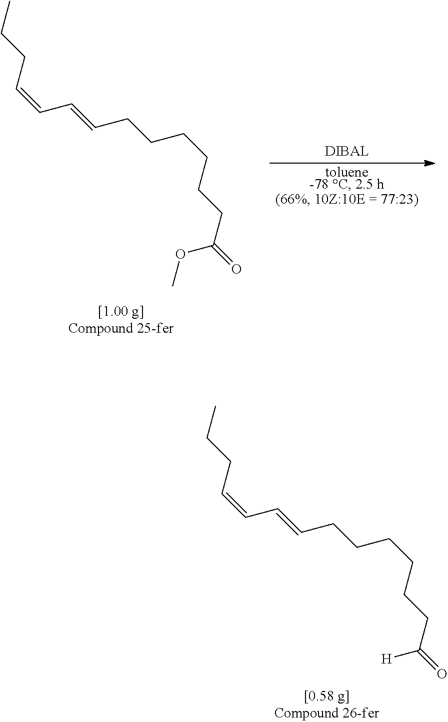

### Compound 25-fer Compound 26-fer

A solution of ester 25-fer (1.00 g, 4.20 mmol, 1 equiv) in anhydrous toluene (21 mL, 0.2 M) was cooled to −78 °C (±2 °C, internal temperature). Then, diisobutylaluminium hydride in hexanes (DIBAL, 4.82 mL, 1.0 M, 4.82 mmol, 1.15 equiv) was added dropwise, while maintaining the internal temperature below −73 °C The mixture was stirred at this temperature for an additional 2 h, when analysis by TLC indicated incomplete conversion. Thus, a further portion of DIBAL in hexanes (0.42 mL, 1.0 M, 0.42 mmol, 0.10 equiv) was added dropwise while maintaining the internal temperature below −73 °C [Note 1] After stirring at this temperature for an additional 20 min, saturated aqueous Rochelle salt solution (50 mL) was added. The well-stirred two-phase system was allowed to warm to 25 °C and stirred at this temperature for 14 h. Then, diethyl ether (30 mL) was added, and the phases were separated. The organic phase was dried over sodium sulfate. The dried solution was filtered, and the filtrate was concentrated under reduced pressure. The residue was purified by flash column chromatography on silica gel (0-20% ethyl acetate in hexanes) to afford diene 26-fer (0.58 g, 2.76 mmol, 66%, 10Z:10E=77:23) as a colorless oil.
[Note 1]: Full conversion is targeted as the starting material has a similar polarity to the desired product. Overreduction to the alcohol does not cause isolation difficulties due to significantly different polarities.

### Compound 26-fer

### Z,E

¹H-NMR (499.64 MHz, CDCl₃): δ=9.76 (t, J=1.8 Hz, 1H), 6.34-6.26 (m, 1H), 5.99-5.90 (m, 1H), 5.63 (dt, J=14.6, 7.0 Hz, 1H), 5.31 (dt, J=10.8, 7.6 Hz, 1H), 2.42 (td, J=7.4, 1.9 Hz, 2H), 2.17-2.00 (m, 5H), 1.62 (ddd, J=11.6, 5.9, 2.7 Hz, 2H), 1.40 (ddt, J=10.6, 7.3, 3.4 Hz, 3H), 1.35-1.29 (m, 4H), 0.90 ppm (dt, J=10.0, 7.4 Hz, 3H).

¹³C-NMR (125.65 MHz, CDCl₃): δ=202.9, 134.3, 130.0, 128.7, 125.8, 43.9, 32.7, 29.7, 29.1, 29.0, 28.9, 22.9, 22.0, 13.8 ppm.

TLC: Rf=0.40 (10% ethyl acetate in hexanes, CAM).

### Example 16

### Synthesis of the Sex Pheromone of Lobesia Botrana

Upscaled, modified versions of our general procedures for the preparation of alkenyl thianthren-5-ium salts and unsaturated carbonyls were used as follows: (1) Tf₂O instead of TFAA and TfOH was used to demonstrate the feasibility of this alternative process; (2) No counterion exchange was performed as triflates are also appropriate starting materials of the TT-Kornblum reaction; (3) NaHCO₃ instead of K₂CO₃ and elevated temperature were used as more suitable conditions to promote the formation of the desired (E)-enal.

### Preparation of diene 27-fer ((7E,9Z)-dodeca-7,9-dien-1-yl acetate)

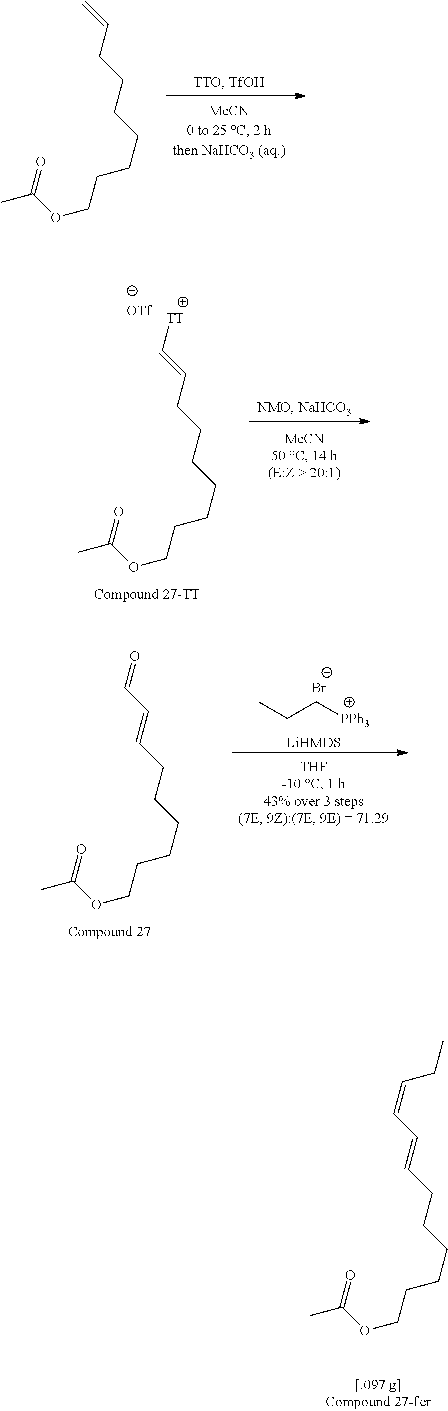

A suspension of 8-nonenyl acetate (1.84 g, 10.0 mmol, 1 equiv) and thianthrene S-oxide (TTO, 2.56 g, 11.0 mmol, 1.10 equiv) in anhydrous acetonitrile (50 mL, 0.2 M) was cooled to 0 °C (internal temperature) under argon atmosphere. Then, trifluoromethanesulfonic anhydride (2.11 mL, 12.5 mmol, 1.25 equiv) was added dropwise, while maintaining the internal temperature below 10 °C An immediate color change of the reaction mixture was observed upon addition and the lilac solution was stirred at 0 °C for a further 1 h. Then, the reaction mixture was warmed to 25 °C and stirred at this temperature for an additional 1 h, when analysis by TLC indicated full conversion. At this point, the reaction mixture was concentrated under reduced pressure and subsequently dissolved in dichloromethane (100 mL). Saturated aqueous sodium bicarbonate solution (100 mL) was added, and the two-phase mixture was vigorously stirred at 25 °C for 15 min. [Note 1] The phases were separated, and the organic phase was dried over sodium sulfate. The dried solution was filtered, and the filtrate was concentrated under reduced pressure to yield the crude 27-TT thianthren-5-ium salt.

### Compound 27-TT

¹H NMR (500 MHz, CDCl₃) δ=8.47 (dd, J=7.8, 1.5 Hz, 2H), 7.83-7.80 (m, 2H), 7.72 (td, J=7.6, 1.5 Hz, 2H), 7.67 (td, J=7.6, 1.5 Hz, 2H), 7.33 (dt, J=14.8, 6.9 Hz, 1H), 6.57 (dt, J=14.8, 1.5 Hz, 1H), 4.01 (t, J=6.7 Hz, 2H), 2.29-2.21 (m, 2H), 2.03 (s, 3H), 1.56 (p, J=6.8 Hz, 2H), 1.41 (h, J=7.1 Hz, 2H), 1.29-1.22 (m, 6H).
[Note 1]: This step promotes the formation of alkenyl thianthrenium salts from the precursor dicationic species.

A solution of the crude alkenyl thianthren-5-ium salt in acetonitrile (100 mL, 0.1 M) was stirred under argon atmosphere at 50 °C To this solution was added N-methylmorpholine N-oxide monohydrate (NMO, 5.41 g, 40 mmol, 4.00 equiv) and well-powdered sodium bicarbonate (4.20 g, 50 mmol, 5.00 equiv) in one portion, and the suspension was stirred at this temperature for 14 h. [Note 2] Then, saturated aqueous sodium bicarbonate solution (200 mL) and diethyl ether (400 mL) were added subsequently. The layers were separated, and the organic phase was washed with saturated aqueous sodium bicarbonate solution (2×200 mL) and brine (200 mL), and was dried over sodium sulfate. The dried solution was filtered, and the filtrate was concentrated under reduced pressure.
[Note 3] The crude 27 enal was used in the next step without further purification.

### Compound 27

¹H NMR (500 MHz, CDCl₃) δ=9.51 (d, J=7.9 Hz, 1H), 6.88-6.79 (m, 1H), 6.12 (ddt, J=15.6, 7.9, 1.5 Hz, 1H), 4.06 (t, J=6.7 Hz, 2H), 2.38-2.29 (m, 2H), 2.05 (s, 3H), 1.71-1.59 (m, 2H), 1.56-1.50 (m, 2H), 1.38 ppm (tq, J=6.1, 2.8 Hz, 4H).
[Note 2]: Technical grade acetonitrile and NMO monohydrate were used as the reaction is not sensitive to water.
[Note 3]: Water bath temperature was set to 30 °C due to possible volatility of the product.

A suspension of propyltriphenylphosphonium bromide (3.85 g, 10.0 mmol, 1.00 equiv) in anhydrous tetrahydrofuran (75 mL) was cooled to −15 °C (±2 °C, internal temperature). Then, lithium bis(trimethylsilyl)amide in tetrahydrofuran (LiHMDS, 10.0 mL, 1.0 M, 10.0 mmol, 1.00 equiv) was added dropwise, while maintaining the internal temperature below −10 °C The bright orange mixture was warmed to 0 °C and stirred at this temperature for an additional 40 min. After cooling back to −15 °C, a solution of the crude enal in anhydrous tetrahydrofuran (25 mL) was added dropwise while maintaining the internal temperature below −10 °C and the mixture was stirred at this temperature for an additional 60 min. At this point, analysis by TLC indicated full conversion, and saturated aqueous sodium bicarbonate solution (200 mL) was added. The well-stirred two-phase system was allowed to warm to 25 °C Then, diethyl ether (400 mL) was added, and the phases were separated. The organic phase was washed with saturated aqueous sodium bicarbonate solution (2×200 mL) and brine (200 mL), and was dried over sodium sulfate. The dried solution was filtered, and the filtrate was concentrated under reduced pressure. The residue was redissolved in hexanes (100 mL), filtered and the precipitate was washed with hexanes. [Note 4] The filtrate was concentrated under reduced pressure and the residue was purified by flash column chromatography on silica gel (0-20% ethyl acetate in hexanes) to afford diene 27-fer (0.97 g, 4.32 mmol, 43%, Z:E=71:29) as a colorless oil.
[Note 4]: The washing was continued until the washing solution did not contain the desired product as indicated by TLC analysis.

### Compound 27-fer

¹H-NMR (499.64 MHz, CDCl₃): δ=6.34-6.25 (m, 1H), 5.90 (tq, J=10.9, 1.3 Hz, 1H), 5.64 (dt, J=14.7, 7.0 Hz, 1H), 5.29 (dt, J=10.8, 7.5 Hz, 1H), 4.04 (td, J=6.7, 2.2 Hz, 2H), 2.17 (pd, J=7.6, 1.6 Hz, 2H), 2.12-2.05 (m, 3H), 2.04 (s, 3H), 1.65-1.56 (m, 3H), 1.43-1.29 (m, 6H), 0.99 ppm (td, J=7.5, 1.6 Hz, 3H).

¹³C-NMR (125.65 MHz, CDCl₃): δ=171.2, 134.3, 131.7, 127.9, 125.6, 64.5, 32.7, 29.2, 28.8, 28.5, 25.7, 21.0 (2C), 14.3 ppm.

TLC: Rf=0.40 (10% ethyl acetate in hexanes, CAM).

### Example 17

### Expanding the Activation of Olefins Towards Industrially Applicable Reagents

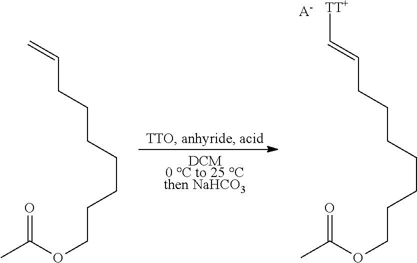

A suspension of 8-nonenyl acetate (184 mg, 1.00 mmol, 1 equiv) and thianthrene S-oxide (TTO, 239 mg, 1.03 mmol, 1.03 equiv) in anhydrous dichloromethane (10 mL, 0.1 M) was cooled to 0 °C under argon atmosphere. At this temperature, carboxylic acid anhydride (3.00 equiv) and sulfonic acid (2.00 equiv) were added dropwise consecutively. An immediate color change of the reaction mixture was observed at the beginning of the first addition step and the lilac/blue solution was stirred at 0 °C for a further 1 h. Then, the reaction mixture was warmed to 25 °C and stirred at this temperature for an additional 1.5-14 h until analysis by TLC indicated full conversion. At this point, saturated aqueous sodium bicarbonate solution (10 mL) was added, and the two-phase mixture was vigorously stirred at 25 °C for 15 min, whereupon a color change to pale yellow was observed and the phases were separated, and the organic phase was dried over sodium sulfate. The dried solution was filtered, and the filtrate was concentrated under reduced pressure to yield the crude alkenyl thiantren-5-ium salts.

Quantitative NMR measurements were carried out using ethylene carbonate as internal standard. The activating agents, reaction times and the corresponding qNMR yields are shown in the table below.

| | | Reaction | | qNMR |
|---|---|---|---|---|
| Anhydride | Sulfonic acid | time | | yield |
| Acetic anhydride | Trifluoromethanesulfonic | 2.5 | h | 75% |
| | acid | | | |
| Trifluoroacetic anhydride | Methanesulfonic acid | 2.5 | h | 55%* |
| Acetic anhydride | Methanesulfonic acid | 14 | h | traces |

*calculated for 40% conversion, as 60% of the starting olefin was still present in the mixture

### Example 18

### Synthesis of the Sex Pheromone of the Red Bollworm Moth

Upscaled, one-pot versions of our general procedures for the preparation of alkenyl thianthren-5-ium salts and unsaturated carbonyls were used as follows: (1) No aqueous work-up was performed in the first step, thianthren-5-ium species were directly transferred into conditions of the TT-Kornblum reaction. (2) The reaction was conducted at 0 °C instead of −20 °C to promote the in-situ transformation of the different thianthrenium species into the alkenyl thianthrenium salt.

### Preparation of diene 28-fer ((Z)-dodeca-9,11-dien-1-yl acetate)

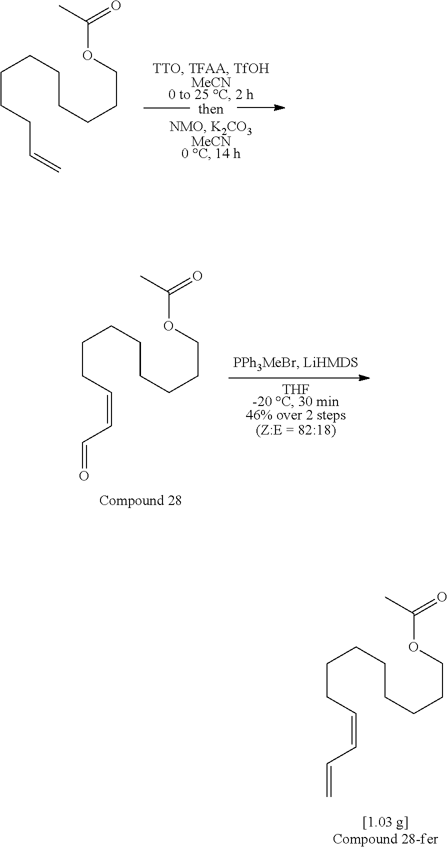

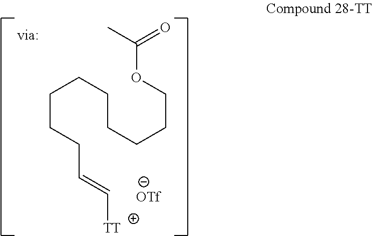

A suspension of undec-10-en-1-yl acetate (2.13 g, 10.0 mmol, 1 equiv) and thianthrene S-oxide (TTO, 2.39 g, 10.3 mmol, 1.03 equiv) in anhydrous acetonitrile (30 mL, 0.3 M) was cooled to 0 °C (internal temperature) under argon atmosphere. Then, trifluoroacetic anhydride (TFAA, 4.24 mL, 40.0 mmol, 3.00 equiv) and trifluoromethanesulfonic acid (1.78 mL, 30.0 mmol, 2.00 equiv) were added consecutively dropwise, while maintaining the internal temperature below 10 °C [Note 1] An immediate color change of the reaction mixture was observed upon the first addition step and the lilac solution was stirred at 0 °C for a further 1 h. Then, the reaction mixture was warmed to 25 °C and stirred at this temperature for an additional 1 h, when analysis by TLC indicated full conversion. At this point, the reaction mixture was concentrated under reduced pressure to yield the crude thianthrenium salt. [Note 2]A small sample was taken out and treated with saturated aqueous sodium bicarbonate solution to yield 28-TT salt for characterization.

### Compound 28-TT

¹H NMR (500 MHz, CDCl₃) δ=8.47 (dd, J=7.8, 1.5 Hz, 2H), 7.83-7.80 (m, 2H), 7.72 (td, J=7.6, 1.5 Hz, 2H), 7.67 (td, J=7.6, 1.5 Hz, 2H), 7.33 (dt, J=14.8, 6.9 Hz, 1H), 6.57 (dt, J=14.8, 1.5 Hz, 1H), 4.01 (t, J=6.7 Hz, 2H), 2.29-2.21 (m, 2H), 2.03 (s, 3H), 1.56 (p, J=6.8 Hz, 2H), 1.41 (h, J=7.1 Hz, 2H), 1.29-1.22 (m, 10H).
[Note 1]: Caution! Special attention should be taken when adding the corrosive trifluoromethanesulfonic acid dropwise at this scale as disposable syringes/needles may disintegrate.
[Note 2]: Water jet vacuum pumps were used with gentle heating by a 30 °C water bath.

A suspension of N-methylmorpholine N-oxide (NMO, 4.69 g, 40 mmol, 4.00 equiv) and well powdered, oven-dried potassium carbonate (6.91 g, 50 mmol, 5.00 equiv) in anhydrous acetonitrile (75 mL) was cooled to 0 °C (±2 °C, internal temperature) and stirred at this temperature for 30 min. [Notes 3,4] Then, a solution of the crude alkenyl thianthren-5-ium salt in anhydrous acetonitrile (25 mL) was added dropwise while maintaining the internal temperature below 5 °C The reaction mixture was stirred at this temperature for 14 h when analysis by TLC indicated full conversion. Then, saturated aqueous sodium bicarbonate solution (400 mL) and dichloromethane (400 mL) were added subsequently. The layers were separated, and the organic phase was washed with saturated aqueous sodium bicarbonate solution (2×200 mL) and dried over sodium sulfate. [Note 5] The dried solution was filtered, and the filtrate was concentrated under reduced pressure. [Note 6] The crude 28 enal was used in the next step without further purification.

### Compound 28

¹H NMR (500 MHz, CDCl₃) δ=10.00 (d, J=8.1 Hz, 1H), 6.57 (dt, J=11.2, 8.2 Hz, 1H), 5.90-5.83 (m, 1H), 3.97 (t, J=6.8 Hz, 2H), 2.53 (q, J=7.5 Hz, 2H), 1.93 (s, 3H), 1.53-1.18 ppm (m, 12H).
[Note 3]: A low-temperature thermostat was used to control cooling bath temperature.
[Note 4]: This preliminary stirring ensures that the internal temperature stabilizes as initial overcooling to ca. −24 °C may occur due to the endothermic dissolution of K₂CO₃.
[Note 5]: These steps eliminate excess NMO and N-alkylated by-products.
[Note 6]: To avoid isomerization of the (Z)-enal, the water bath temperature was set to 30 °C

A suspension of methyltriphenylphosphonium bromide (2.68 g, 7.5 mmol, 0.75 equiv) in anhydrous tetrahydrofuran (46.7 mL) was cooled to −20 °C (±2 °C, internal temperature). Then, lithium bis(trimethylsilyl)amide in tetrahydrofuran (LiHMD S, 7.5 mL, 1.0 M, 7.5 mmol, 0.75 equiv) was added dropwise, while maintaining the internal temperature below −15 °C and the bright yellow mixture was stirred for an additional 30 min. Then, the mixture was warmed to 0 °C and stirred at this temperature for an additional 30 min. After cooling back to −20 °C, a solution of the crude enal in anhydrous tetrahydrofuran (20 mL) was added dropwise while maintaining the internal temperature below −15 °C and the mixture was stirred for an additional 30 min. At this point, saturated aqueous sodium bicarbonate solution (200 mL) was added, and the two-phase system was allowed to warm to 25 °C Then, dichloromethane (400 mL) was added, and the phases were separated. The aqueous phase was extracted with dichloromethane (2×200 mL). The combined organic phases were washed with brine (300 mL) and dried over sodium sulfate. The dried solution was filtered, and the filtrate was concentrated under reduced pressure. The residue was purified by flash column chromatography on silica gel (0-20% ethyl acetate in hexanes) to afford diene 28-fer (1.03 g, 4.57 mmol, 46%, Z:E=82:18) as a colorless oil.

### Compound 28-fer

¹H-NMR (499.64 MHz, CDCl₃): δ=6.64-6.54 (m, 1H), 5.98-5.92 (m, 1H), 5.44-5.37 (m, 1H), 5.17-5.09 (m, 1H), 5.03 (dd, J=10.2, 2.0 Hz, 1H), 4.11-3.97 (m, 2H), 2.18-2.10 (m, 2H), 2.00 (s, 3H), 1.57 (p, J=6.8 Hz, 2H), 1.41-1.22 ppm (m, 10H).

¹³C-NMR (125.65 MHz, CDCl₃): δ=170.9, 132.7, 132.1, 129.1, 116.5, 64.4, 29.4, 29.2, 29.0, 29.0, 28.4, 27.5, 25.7, 20.8k ppm.

TLC: Rf=0.40 (10% ethyl acetate in hexanes, CAM).

### Example 19

### Synthesis of the Pear Ester Kairomone and Fragrance

We implemented a slightly modified version of Wickens' cation pool approach (ref xxiii) for the electrochemical oxidation of 1-octene using thianthrene. The formed thianthren-5-ium adducts were directly transferred into the conditions of the TT-Kornblum reaction, where a modified version of our general procedure for the preparation of unsaturated carbonyls was used as follows: (1) 2-MeTHF was used as a solvent instead of MeCN for easier removal of the electrolyte salt and because of its slightly higher volatility; (2) 10 equiv. of K₂CO₃ was used instead of 5 equiv, and the reaction was conducted at −10 °C instead of −20 °C to promote the in situ transformation of the different thianthrenium species into the alkenyl thianthren-5-ium salt. Finally, without isolation, the obtained enal compound was subjected to a Wittig reaction according to a modified version of Bestmann's procedure (ref xxiv) to obtain the pear ester.

### Preparation of ester 29-fer (ethyl (2E,4Z)-deca-2,4-dienoate)

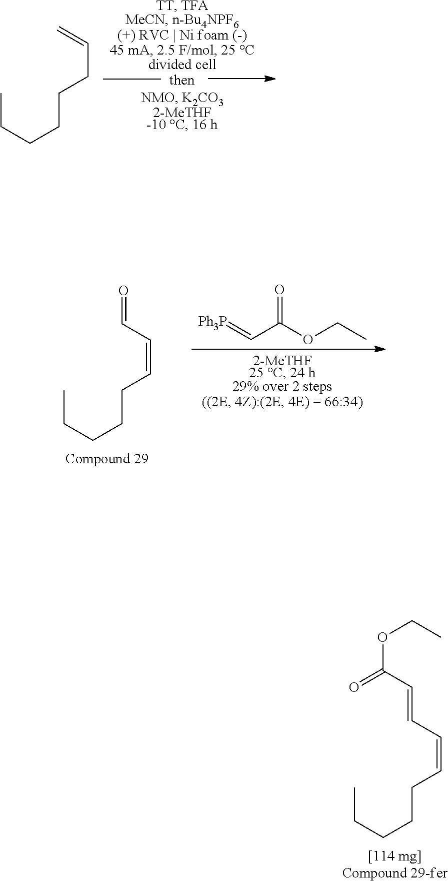

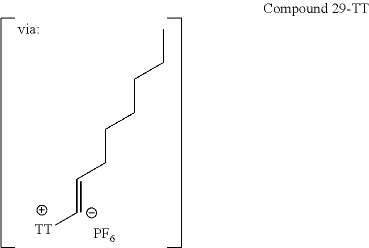

Trifluoroacetic acid (770 μL, 5 equiv, 10.0 mmol) was added to a solution of tetra-n-butylammonium hexafluorophosphate (620 mg, 0.8 equiv, 1.60 mmol) in anhydrous acetonitrile (7 mL, 0.2 M). This solution was transferred to the cathodic compartment of an IKA Pro-Divide divided electrochemical cell equipped with magnetic stirring bars. Next, 1-octene (224 mg, 1 equiv, 2.00 mmol) was added to a separate solution of tetra-n-butylammonium hexafluorophosphate (620 mg, 0.8 equiv, 1.60 mmol) in anhydrous acetonitrile (7 mL, 0.2 M). This solution in turn was transferred to the anodic compartment of the electrochemical cell. Finally, under stirring, thianthrene (TT, 649 mg, 1.5 equiv, 3.00 mmol) was added to the anodic compartment [Note 1]. The closing cap of the cell was equipped with a standard IKA RVC anode and a standard IKA nickel foam cathode, and the cell was sealed. Both compartments were flushed with argon gas through the septa on the top of the cell, after which argon-filled balloons were inserted into them. The cell was inserted into the IKA ElectraSyn 2.0 potentiostat and the reaction mixture was electrolyzed at a constant current of 45 mA for 2.5 F/mol of alkene, with stirring set to 750 RPM [Note 2]. Complete conversion of the alkene was achieved based on TLC and ¹H NMR measurement. Next, the cell was opened, and the lilac anodic solution was transferred into a 50 mL flask. The RVC anode and the anodic compartment were further washed with 7 mL anhydrous acetonitrile into the flask. Then, under reduced pressure, this anodic reaction mixture was evaporated to dryness to yield a crude mixture of different thianthrenium adducts. A small sample was taken out and treated with saturated aqueous sodium bicarbonate solution to yield 29-TT salt for characterization.

### Compound 29-TT

¹H NMR (500 MHz, cdcl₃) δ 8.39 (dd, J=7.8, 1.5 Hz, 2H), 7.82 (dd, J=7.8, 1.4 Hz, 2H), 7.73 (td, J=7.7, 1.4 Hz, 2H), 7.67 (td, J=7.7, 1.4 Hz, 2H), 7.30 (dd, J=14.8, 7.0 Hz, 1H), 6.56 (dt, J=14.7, 1.5 Hz, 1H), 2.29-2.20 (m, 2H), 1.40 (t, J=7.7 Hz, 2H), 1.21 (td, J=6.6, 3.8 Hz, 6H), 0.84-0.78 (m, 3H).

To this flask, a magnetic stirring bar and 20 mL anhydrous 2-methyltetrahydrofuran (0.1 M) were added, and the contents of the flask were suspended using sonication. The flask was flushed with argon gas, closed with a rubber septum, and equipped with an argon-filled balloon. Under stirring, the reaction mixture was cooled to −10 °C, and potassium carbonate (2.76 g, 10 equiv, 20.0 mmol) and N-methylmorpholine N-oxide (NMO, 937 mg, 4 equiv, 8.00 mmol) were added to it in one portion. Almost immediately, the intensive lilac color of the suspension started to fade and transform into a pale-yellow color. The resulting suspension was stirred for further 16 hours at −10 °C, after which complete conversion of the alkenyl thianthrenium salt could be observed using TLC and ¹H NMR measurement. Next, while still cold, an aqueous 10% citric acid solution was added to the reaction mixture until neutral or slightly acidic pH is achieved (approx. 40-50 mL). The phases were separated [Note 3], and the organic layer was further washed with 2×30 mL brine. Then, the organic phase was dried over sodium sulfate and filtered, to yield a solution of the crude 29 enal, which was used in the next synthetic step without further purification.

### Compound 29

¹H NMR (500 MHz, CDCl₃) δ=9.55 (d, J=8.1 Hz, 1H), 6.65 (dt, J=11.5, 8.3 Hz, 1H), 5.95 (m, J=11.2, 8.1 Hz, 1H), 2.40-2.80 (m, 2H), 1.05-1.08 (m, 6H), 0.93 ppm (t, J=6.2 Hz, 3H).

In the final synthetic step, ethyl 2-(triphenyl-λ⁵-phosphaneylidene)acetate (766 mg, 1.1 equiv, 2.20 mmol) was added into an argon-flushed 50 mL flask equipped with a magnetic stirring bar, rubber septum, and an argon-filled balloon. The solution of the crude enal in 2-methyltetrahydrofuran (20 mL, 0.1 M) was added to the flask, and the reaction mixture was stirred at 25 °C for 24 hours until complete conversion of the enal could be observed using TLC and ¹H NMR measurement. Next, the reaction mixture was concentrated under reduced pressure [Note 4] and 5 mL hexane was added. The resulting suspension was sonicated for 10 minutes and filtered. The filtrate containing the crude product was subjected to flash column chromatography on silica gel (gradient elution, 0-10% ethyl acetate in hexanes) [Note 5] to afford the pear ester (29-fer) (114 mg, 581 μmol, 29%, (2E,4Z):(2E,4E)=66:34) as a colorless oil.
[Note 1]: As the solubility of thianthrene is low in acetonitrile, complete dissolution of the solids will take place only as the electrochemical reaction consumes the thianthrene. Intensive stirring is needed to suspend the thianthrene so that the stirring bars of the Pro-Divide cell don't get stuck in the solid material.
[Note 2]: The IKA ElectraSyn was set up as follows: New experiments→Constant current→45 mA→No reference electrode→Total charge→2.0 mmol, 2.5 F/mol→No alternating polarity→Start→750 RPM stirring
[Note 3]: In some cases, a part of the supporting electrolyte precipitated during the extraction steps producing a layer of precipitate between the organic and aqueous phase which made their separation cumbersome. Filtering of the biphasic mixture through pleated filter paper helped in these cases.
[Note 4]: Due to the volatility of the pear ester, extra care should be taken during the evaporations under reduced pressure. Using a warmer water bath (50-60 °C) and higher pressures (>300 mbar) usually gave better results.
[Note 5]: The remaining supporting electrolyte and residual polar solvents may cause the product to elute with the solvent front. In these cases, the fractions containing the product should be concentrated under reduced pressure and the flash chromatography should be repeated.

### Compound 29-fer

¹H-NMR (499.64 MHz, CDCl₃): δ=7.60 (ddd, J=15.2, 11.7, 1.1 Hz, 1H), 6.18-6.08 (m, 2H), 5.85 (d, J=15.0 Hz, 1H), 4.19 (dq, J=8.9, 7.1 Hz, 2H), 2.29 (qd, J=7.6, 1.6 Hz, 2H), 1.44-1.38 (m, 2H), 1.33-1.24 (m, 7H), 0.89-0.86 ppm (m, 3H).

¹³C-NMR (125.65 MHz, CDCl₃): δ=167.3, 141.7, 139.5, 126.4, 121.1, 60.2, 31.3, 29.0, 28.2, 22.5, 14.3, 14.0 ppm.

TLC: Rf=0.43 (10% ethyl acetate in hexanes, 254 nm UV light or CAM).

The acquired spectral data was in accordance with the previously reported results.(ref xxiv)

### Example 19

### Synthesis of the pheromone (9Z, 11Z)-9,11-tetradecadienol

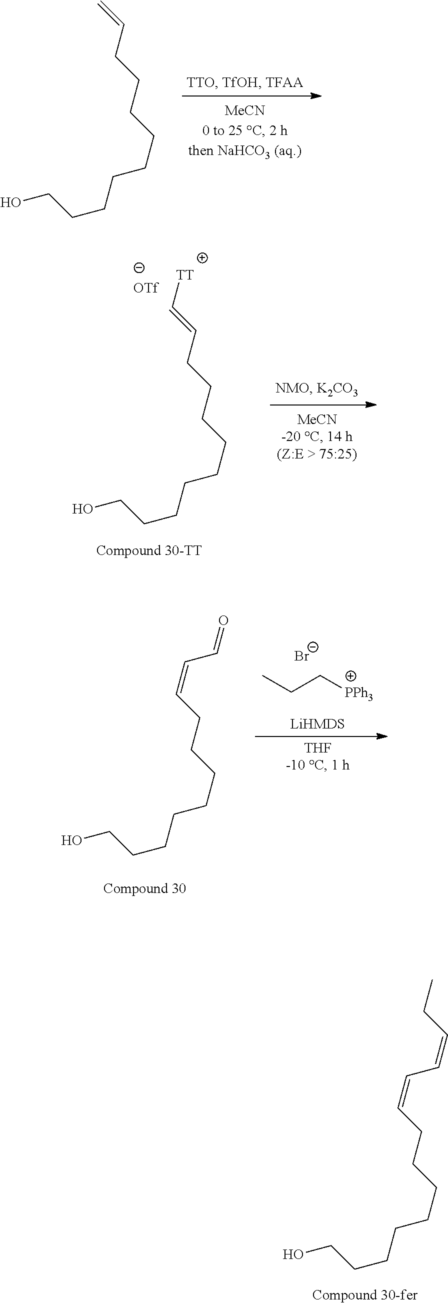

A suspension of undec-10-en-1-ol (1.70 g, 10.0 mmol, 1 equiv) and thianthrene S-oxide (TTO, 2.39 g, 10.3 mmol, 1.03 equiv) in anhydrous acetonitrile (100 mL, 0.1 M) was cooled to 0 °C (internal temperature) under argon atmosphere. Then, trifluoroacetic anhydride (TFAA, 3.18 ml, 30.0 mmol, 3.00 equiv) and trifluoromethanesulfonic acid (1.18 mL, 20.0 mmol, 2.00 equiv) were added consecutively dropwise, while maintaining the internal temperature below 10 °C An immediate color change of the reaction mixture was observed upon addition and the lilac solution was stirred at 0 °C for a further 1 h. Then, the reaction mixture was warmed to 25 °C and stirred at this temperature for an additional 1 h, when analysis by TLC indicated full conversion. Saturated aqueous sodium bicarbonate solution (100 mL) was added, and the two-phase mixture was vigorously stirred at 25 °C for 15 min. The phases were separated, and the organic phase was dried over sodium sulfate. The dried solution was filtered, and the filtrate was concentrated under reduced pressure to yield the crude mixture of 30-TT thianthren-5-ium salt and the corresponding trifluoroacetyl ester. 100-100 mL of isopropanol and saturated sodium bicarbonate solution was added and the mixture was stirred until TLC indicated complete hydrolyzation of the ester. Then 250 mL dichloromethane was added, and the phases were separated. The organic phase was washed with 100 mL brine, then dried over sodium sulfate and then concentrated under reduced pressure to give the crude 30-TT thianthren-5-ium salt.

### Compound 30-TT

¹H NMR (499.64 MHz, CDCl₃) δ=8.25 (dd, J=7.8, 1.4 Hz, 2H), 7.89 (dd, J=8.0, 1.4 Hz, 2H), 7.78 (td, J=7.6, 1.5 Hz, 2H), 7.70 (td, J=7.6, 1.5 Hz, 2H), 7.11 (dt, J=14.3, 6.9 Hz, 1H), 6.61-6.50 (m, 1H), 3.56 (t, J=6.6 Hz, 2H), 2.33-2.21 (m, 2H), 1.58-1.20 (m, 14H).

A solution of the crude alkenyl thianthren-5-ium salt in acetonitrile (100 mL, 0.1 M) was stirred under argon atmosphere at −20 °C To this solution was added N-methylmorpholine N-oxide monohydrate (NMO, 5.41 g, 40 mmol, 4.00 equiv) and well-powdered potassium carbonate (6.91 g, 50 mmol, 5.00 equiv) in one portion, and the suspension was stirred at this temperature for 14 h. [Note 2] Then, saturated aqueous sodium bicarbonate solution (200 mL) and diethyl ether (400 mL) were added subsequently. The layers were separated, and the organic phase was washed with saturated aqueous sodium bicarbonate solution (2×200 mL) and brine (200 mL), and was dried over sodium sulfate. The dried solution was filtered, and the filtrate was concentrated under reduced pressure.
[Note 3] The crude 30 enal was used in the next step without further purification.

### Compound 30

¹H-NMR (499.64 MHz, CDCl₃): δ=10.08 (d, J=8.1 Hz, 1H), 6.61 (dt, J=11.2, 8.2 Hz, 1H), 5.98-5.94 (m, 1H), 3.63 (t, J=6.6 Hz, 2H), 2.59 (qd, J=7.7, 1.5 Hz, 2H), 1.96 (br s, 1H), 1.55 (t, J=6.8 Hz, 2H), 1.51 (t, J=7.1 Hz, 2H), 1.40-1.32 ppm (m, 8H).
[Note 2]: Technical grade acetonitrile and NMO monohydrate were used as the reaction is not sensitive to water.
[Note 3]: Water bath temperature was set to 30 °C due to possible volatility of the product.

A suspension of propyltriphenylphosphonium bromide (3.85 g, 10.0 mmol, 1.00 equiv) in anhydrous tetrahydrofuran (75 mL) was cooled to −15 °C (±2 °C, internal temperature). Then, lithium bis(trimethylsilyl)amide in tetrahydrofuran (LiHMDS, 10.0 mL, 1.0 M, 10.0 mmol, 1.00 equiv) was added dropwise, while maintaining the internal temperature below −10 °C The bright orange mixture was warmed to 0 °C and stirred at this temperature for an additional 40 min. After cooling back to −15 °C, a solution of the crude enal in anhydrous tetrahydrofuran (25 mL) was added dropwise while maintaining the internal temperature below −10 °C and the mixture was stirred at this temperature for an additional 60 min. At this point, analysis by TLC indicated full conversion, and saturated aqueous sodium bicarbonate solution (200 mL) was added. The well-stirred two-phase system was allowed to warm to 25 °C Then, diethyl ether (400 mL) was added, and the phases were separated. The organic phase was washed with saturated aqueous sodium bicarbonate solution (2×200 mL) and brine (200 mL), and was dried over sodium sulfate. The dried solution was filtered, and the filtrate was concentrated under reduced pressure. The residue was redissolved in hexanes (100 mL), filtered and the precipitate was washed with hexanes to yield the crude diene 30-fer with 20% overall qNMR yield for the three steps (using ethylene carbonate as internal standard). Spectral properties were in accordance with those reported in the literature. (ref xxv)

### Example 20

### ONE-pot oxidation via activation using diphenyl sulfoxide

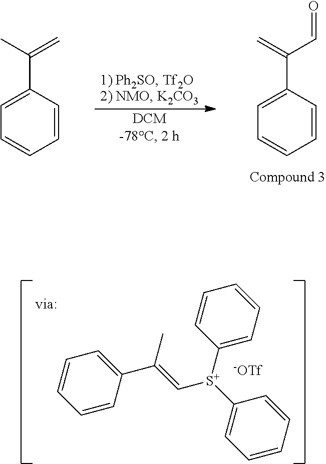

A suspension of a-methylstyrene (118 mg, 1.00 mmol, 1 equiv) in anhydrous dichloromethane (10 mL, 0.1 M) was cooled to −78 °C (internal temperature) under argon atmosphere. Then, diphenyl sulfoxide (243 mg, 1.20 mmol, 1.2 equiv) and trifluoromethanesulfonic anhydride (2.11 mL, 12.5 mmol, 1.25 equiv) was added subsequently, while maintaining the internal temperature below −70 °C, and the solution was stirred at −78 °C for a further 1 h. Then, N-methylmorpholine N-oxide monohydrate (NMO, 541 mg, 4.0 mmol, 4.00 equiv) and well-powdered potassium carbonate (691 mg, 5.0 mmol, 5.00 equiv) were added in one portion, and the suspension was stirred and let to warm up to 25 °C overnight. Then, saturated aqueous sodium bicarbonate solution (10 mL) was added, the phases were separated, and the organic phase was dried over sodium sulfate. The dried solution was filtered, and the filtrate was concentrated under reduced pressure to give the crude 3 enal with 20% qNMR yield (using ethylene carbonate as internal standard). Spectral properties were in accordance with those reported in the literature. (ref xv)

### Example 21

### Preparation and TT-Kornblum/Ganem oxidation of oxidized alkenyl thianthrenium (TTOO) Salts

### Preparation of (E)-5-(6-(1,3-dioxoisoindolin-2-yl)hex-1-en-1-yl)-5H-thianthren-5-ium 10,10-dioxide tetrafluoroborate (Compound 8-TTOO)

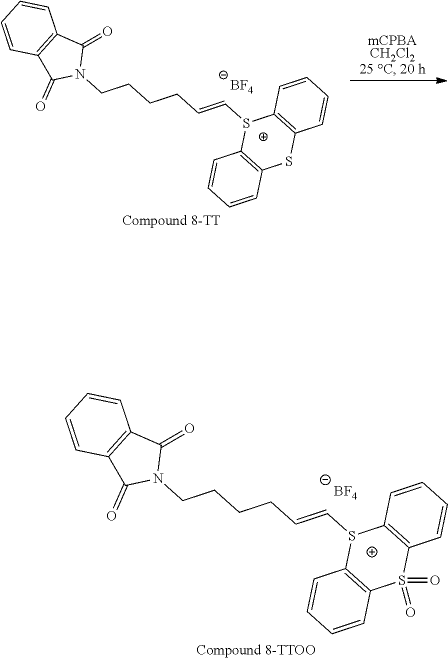

To a solution of compound 8-TT (500 mg, 941 μmol, 1 equiv) in dichloromethane (4.7 mL, 0.2 M) was added meta-chloroperoxybenzoic acid (70% purity, 510 mg, 2.07 mmol, 2.2 equiv) in one portion at 25 °C Then, the reaction mixture was stirred at this temperature for 20 h, when analysis by LC-MS indicated full conversion. At this point, the reaction mixture was concentrated under reduced pressure onto Celite support to yield the crude oxidized thianthrenium salt Compound 8-TTOO. Purification by flash column chromatography on flash silica gel (0% isopropanol in dichloromethane grading to 15% isopropanol in dichloromethane) afforded the title compound 8-TTOO (210 mg, 373 μmol, 40%) as a white solid.

### Compound 8-TTOO

¹H NMR (499.64 MHz, CDCl₃) δ=8.46 (dd, J=7.7, 1.3 Hz, 2H), 8.34 (dd, J=7.5, 1.6 Hz, 2H), 8.04-7.94 (m, 4H), 7.92-7.85 (m, 1H), 7.77 (dd, J=5.5, 3.0 Hz, 2H), 7.68 (dd, J=5.5, 3.0 Hz, 2H), 6.99 (dt, J=14.7, 1.5 Hz, 1H), 3.63 (t, J=6.9 Hz, 2H), 2.55 (q, J=6.6 Hz, 2H), 1.69 (dt, J=14.7, 6.8 Hz, 2H), 1.61 ppm (p, J=7.4 Hz, 2H).

¹³C-NMR (125.65 MHz, CDCl₃): δ=168.3, 163.9, 138.0, 135.6, 135.1, 134.0, 133.9, 132.0, 127.8, 124.7, 123.2, 113.9, 37.1, 33.4, 27.8, 24.2 ppm.

LCMS (ESI): calcd. for C₂₆H₂₂NO₄S₂⁺ M⁺ 476, found 476.

### Procedures for Thianthrenium-Kornblum/Ganem Oxidations Starting from TTOO Salts

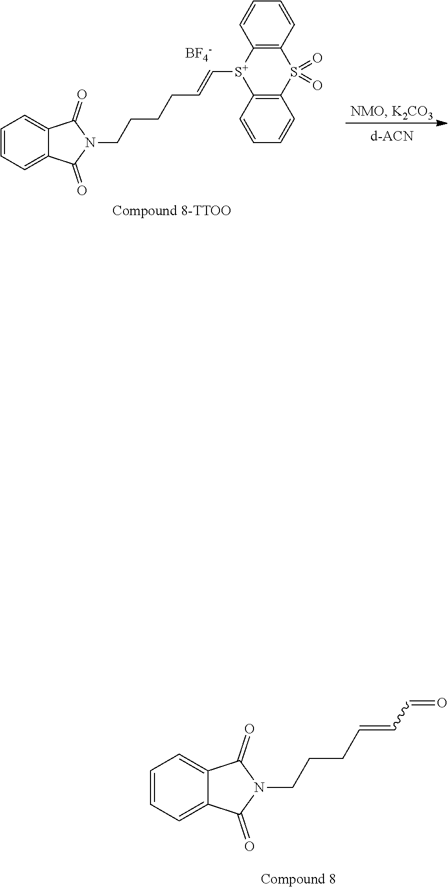

In a 5 mm NMR tube NMO (29 mg, 0.25 mmol, 4.0 equiv) and K₂CO₃ (43 mg, 0.31 mmol, 5.0 equiv) were suspense in d-ACN (200 mL) and stirred at −20 °C or −40 °C for 5 minutes, then a solution of Compound 8-TTOO (35 mg, 62 μmol, 1 equiv) in d-ACN (420 μL) was added, and the reaction mixture was stirred a) at −20 °C for 1 hour, or b) at −40 °C for 16 hours. To quantify our results, q-¹HNMR measurements were carried out and we used ethylene carbonate as an internal standard.

In case a) after 1 h, full conversion was observed, and the NMR yield of the reaction was 42% for the Z product and 12% for the E product.

In case b) after 16 h, full conversion was observed, and the NMR yield of the reaction was 52% for the Z product and 12% for the E product.

These results demonstrate, that due to the increased reactivity of the TTOO salts compared to the TT-salts, lower temperatures can be used for the oxidation reactions, which leads to a better selectivity towards the Z product.

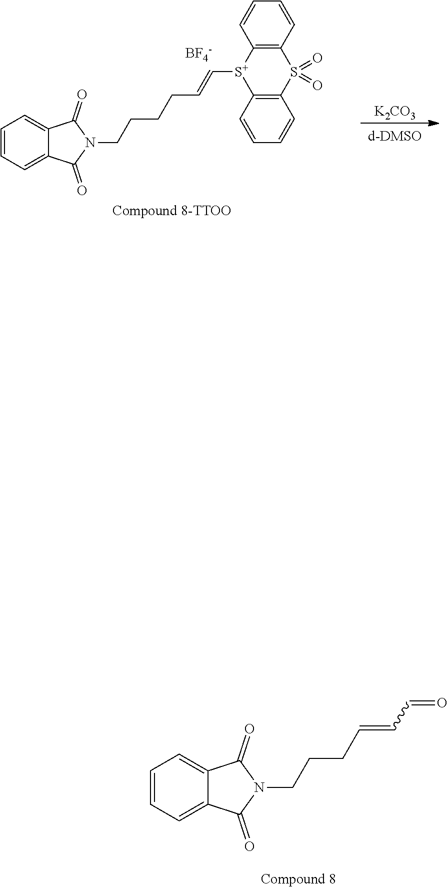

To a solution of Compound 8-TTOO (35 mg, 62 μmol, 1 equiv) in d-DMSO (0.62 mL, 0.1 M) was added K₂CO₃ (43 mg, 0.31 mmol, 5.0 equiv) and the suspension was stirred at 25 °C for 20 minutes, when TLC analysis indicated full conversion. The solution was filtered and q-¹HNMR measurements were carried out to quantify our results while using ethylene carbonate as an internal standard. The NMR 15 yield of the reaction was 51% for the Z product and 20% for the E product.

These results demonstrate, that due to the increased reactivity of the TTOO salts compared to the TT-salts, sulfoxide (i.e. DMSO) mediated oxidations become faster and more selective, thus higher yields can be observed.

### REFERENCES

i) K. M. Wampler, C. W. Lee, D. Rozzell, Synthesis of Conjugated Diene Pheromones and Related Compounds, 2020, 2020123534:A1.
ii) B. L. Quigley, R. H. Grubbs, Chem. Sci. 2014, 5, 501-506.
iii) (a) C. Li, M. Li, J. Li, J. Liao, W. Wu, H. Jiang, J. Org. Chem. 2017, 82, 10912-10919.; (b) C. Li, H. Chen, J. Li, M. Li, J. Liao, W. Wu, H. Jiang, Adv. Synth. Catal. 2018, 360, 1600-1604.; (c) J. Muzart, P. Pale, J.-P. Pete, Tetrahedron Lett. 1982, 23, 3577-3578.; (e) O. Vakuliuk, F. G. Mutti, M. Lara, D. T. Gryko, W. Kroutil, Tetrahedron Lett. 2011, 52, 3555-3557.; (f) T. Wang, S.-K. Xiang, C. Qin, J.-A. Ma, L.-H. Zhang, N. Jiao, Tetrahedron Lett. 2011, 52, 3208-3211.; (g) X. Xing, N. R. O'Connor, B. M. Stoltz, Angew. Chem. Int. Ed. 2015, 54, 11186-11190.
iv) Kozhuskov, S. I.; Alcarazo, M. Eur. J. Inorg. Chem. 2020, 2486. S. I. Kozhushkov, M. Alcarazo, Eur. J Inorg. Chem. 2020, 2020, 2486-2500.
v) (a) J.-I. Matsuo, H. Yamanaka, A. Kawana, T. Mukaiyama, Chem. Lett. 2003, 32, 392-393.; (b) H. Yamanaka, J.-I. Matsuo, A. Kawana, T. Mukaiyama, Chem. Lett. 2003, 32, 626-627.; (c) H. Yamanaka, T. Mukaiyama, Chem. Lett. 2003, 32, 1192-1193.; (d) H. J. Shine, B. K. Bandlish, S. R. Mani, A. G. Padilla, J Org. Chem. 1979, 44, 915-917.
vi) (a) J. Chen, J. Li, M. B. Plutschack, F. Berger, T. Ritter, Angew. Chem. Int. Ed Engl. 2020, 59, 5616-5620.; (b) T. Ritter, F. Berger, Reagents and Process for Direct c-H Functionalization, 2020, 2020094673:A1.
vii) J. Chen, Regio- and Stereoselective Thianthrenation of Olefins to Access Versatile Alkenyl Electrophiles and Fluorination of Aryl Thianthrenium Salts, RWTH Aachen, PhD Thesis, 2020
viii) (a) D. E. Holst, D. J. Wang, M. J. Kim, I A. Guzei, Z. K. Wickens, Nature 2021, 596, 74-79.; (b) D. J. Wang, K. Targos, Z. K. Wickens, J Am. Chem. Soc. 2021, 143, 21503-21510.
ix) (a) M.-S. Liu, H.-W. Du, W. Shu, Chem. Sci. 2022, 13, 1003-1008.; (b) W. Shu, M.-S. Liu, Allylation Coupling Reaction Method and Application Thereof, 2021, CN:113292422:A.
x) N. Kornblum, J. W. Powers, G. J. Anderson, W. J. Jones, H. O. Larson, O. Levand, W. M. Weaver, J Am. Chem. Soc. 1957, 79, 6562-6562.
xi) L. Kurti, B. Czako, Strategic Applications of Named Reactions in Organic Synthesis, Elsevier, 2005.
xii) A. G. Godfrey, B. Ganem, Tetrahedron Lett. 1990, 31, 4825-4826.
xiii) J. Forrester, R. V. H. Jones, L. Newton, P. N. Preston, Tetrahedron 2001, 57, 2871-2884.
xiv) K. E. O'Shea, C. S. Foote, J Am. Chem. Soc. 1988, 110, 7167-7170.
xv) Q. Zhu, Y. Luo, Y. Guo, Y. Zhang, Y. Tao, J. Org. Chem. 2021, 86, 5463-5476.
xvi) B. Zhou, Q. Wu, Z. Dong, J. Xu, Z. Yang, Org. Lett. 2019, 21, 3594-3599.
xvii) M. Stephan, J. Panther, F. Wilbert, P. Ozog, T. J. J. Miller, European J Org. Chem. 2020, 2020, 2086-2092.
xviii) J. Zhang, L. Wang, Q. Liu, Z. Yang, Y. Huang, Chem. Commun. 2013, 49, 11662-11664.
xix) L.-Q. Cui, K. Liu, C. Zhang, Org. Biomol. Chem. 2011, 9, 2258-2265.
xx) a) D. A. Oare, M. A. Henderson, M. A. Sanner, C. H. Heathcock, J Org. Chem. 1990, 55, 132-157. b) S. Nakatsu, A. T. Gubaidullin, V. A. Mamedov, S. Tsuboi, Tetrahedron 2004, 60, 2337-2349.
xxi) (a) J. Chen, J. Li, M. B. Plutschack, F. Berger, T. Ritter, Angew. Chem. 2020, 132, 5665-5669.; Angew. Chem. Int. Ed. 2020, 59, 5616-5620 (b) M.-S. Liu, H.-W. Du, W. Shu, Chem. Sci. 2022, 13, 1003-1008.
xxii) J. Chen, J. Li, M. B. Plutschack, F. Berger, T. Ritter, Angew. Chem. 2020, 132, 5665-5669.; Angew. Chem. Int. Ed. 2020, 59, 5616-5620.
xxiii) D. J. Wang, K. Targos, Z. K. Wickens, J Am. Chem. Soc. 2021, 143, 21503-21510.
xxiv) H. J. Bestmann, J. Stip, Liebigs Ann. Chem. 1982, 1982, 363-365
xxv) Y. Miyake, T. Kinsho, M. Yamashita, N. Ishibashi, Y. Nagae, A. Baba, Method for Producing Asymmetric Conjugated Diyne Compound and Method for Producing Z,Z-Conjugated Diene Compound Using the Same, 2016, 20160229829:A1.

1. Method for preparing α,β-unsaturated carbonyls according to Formula I: 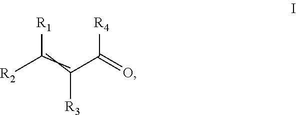
wherein
R1 is H; C1-C20 alkyl, preferably C1-C14 alkyl; C2-C20 alkenyl, preferably C2-C14 alkenyl; C2-C20 alkynyl, preferably C2-C14 alkynyl; —O—C1-C20 alkyl, preferably —O—C1-C14 alkyl; —O—C2-C20 alkenyl; —O—C2-C20 alkynyl; C3-C10 cycloalkyl; 4-10-membered heterocycloalkyl containing one or more heteroatoms independently selected from N, O and S; C6-C14 aryl, preferably phenyl; or 5-10-membered heteroaryl containing 1-3 heteroatoms independently selected from N, O and S;
wherein each alkyl, alkenyl and alkynyl can be straight chained or branched, and each alkyl, alkenyl, alkynyl, cycloalkyl, heterocycloalkyl, heteroaryl and aryl can be substituted with 1-3 substituents independently selected from halogen, —CN, —OH, —OR25, —OTs, —OMs, —O—C(O)R21, ═O, —O—C1-C6 alkyl, —COOH, —C(O)—OR22, 1,3-dioxoisoindolin-2-yl, —NH—C(O)—C1-C6 alkyl, —N(C1-C6 alkyl)-C(O)—C1-C6 alkyl, —NHR23, —N(C1-C6 alkyl)R23, —C(O)—NH—C1-C6 alkyl, —C(O)—N(C1-C6 alkyl)₂, C3-C10 cycloalkyl, 4-10-membered heterocycloalkyl containing one or more heteroatoms independently selected from N, O and S, C6-C10 aryl, 5-10-membered heteroaryl containing 1-3 heteroatom independently selected from N, O and S; or
R1 and R2 together with the carbon atom to which they are attached may form a 5-6 membered cycloalkyl which is preferably saturated; or
R1 and R4 together with the carbon atoms to which they are attached may form a partially unsaturated 5-15-membered, preferably 5-8-membered cycloalkyl; or R1 and R4 may form a chain having 2-5 chain atoms and comprises CH2 units and/or 1-2 heteroatoms selected from N, O and S;
R2 is H, or together with R1, R2 may form cycloalkyl as described above;
R3 is H, C1-C4 alkyl, C6-C10 aryl, 4-methyltetrahydro-2H-pyran-2-yl;
with the proviso that R1 is other than H when all of R2-R4 are H
R4 is H, C1-C4 alkyl, 4-methyltetrahydro-2H-pyran-2-yl; or together with R1, R4 may form cycloalkyl or chain as described above;
R21 is H; C1-C4 alkyl; or C6-C10 aryl, preferably phenyl;
R22 is C1-C4 alkyl, C2-C4 alkenyl or C2-C4 alkynyl;
R23 is N-protecting group;
R25 is O-protecting group;
wherein the methods include the following step:
(i) reacting an alkenyl sulfonium salt of Formula III-1 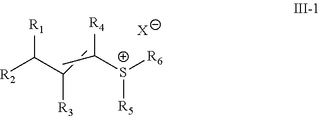
or a synthetic equivalent product thereof including
an allyl-sulfonium of Formula III-2 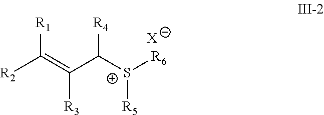
or a bis-adduct of Formula III-3 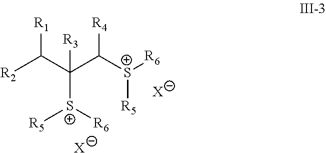
or a dicationic adduct of Formula III-4 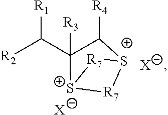
III-4
or a mixture thereof;
wherein R1-R4 has the meaning as described above,
R5 and R6 are selected from C1-C6 alkyl, C5-C7 cycloalkyl, C6-C10 aryl, wherein each of alkyl, cycloalkyl and aryl can be substituted with one or more substituents independently selected from halogen, C1-C2 alkyl, or a —(CH2)ₙ-heteroatom-(CH2)ₘH chain where n and m is independently 0 or 1 and the heteroatom is preferably selected from N, O and S, more preferably S or oxidized heteroatom (e.g. SO, SO2, more preferably SO2); preferably R5 and R6 are C6-C10 aryl, more preferably both aryl has at least one substituent in ortho position and said substituents of the aryls (R5 and R6) may form a direct bond between the aryls or a chain comprising CH2 units and/or a heteroatom (preferably selected from N, O and S, more preferably S), or oxidized heteroatom (e.g. SO, SO2, more preferably SO2)
more preferably R5 and R6 are both an optionally substituted phenyl
even more preferably the SR5R6 moiety is 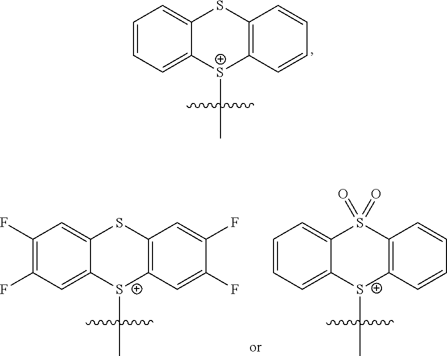 [SMILES: *[S+]1c2ccccc2Sc2ccccc21 ; *[S+]1c2cc(F)c(F)cc2Sc2cc(F)c(F)cc21 ; *[S+]1c2ccccc2S(=O)(=O)c2ccccc21]
X— is a suitable anion, preferably TfO⁻, MsO⁻, TsO⁻, TFA⁻, BF₄⁻, PF₆⁻, ClO₄⁻,
both R7 is ortho-phenylene which is optionally substituted as described above at R5 and R6;
with a nucleophilic oxidizing agent, preferably an amine-oxide or sulfoxide according to Formula IIIa or IIIb: 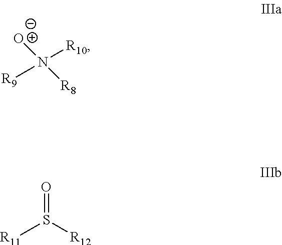
wherein R8, R9, and R10 are independently selected from C1-C4 alkyl, phenyl, or
R8 and R9 together with N atom to which they are attached, or R8-R10 together with N atom to which they are attached, may form a saturated, partially unsaturated or aromatic heterocyclic ring having 5-7 ring forming atom, and comprising a N atom and may further comprise a heteroatom selected from N, O and S,
preferably, Formula IIIa is 4-Methylmorpholine N-oxide 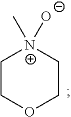
pyridine N-oxide or substituted pyridine N-oxide, for example 2,6-dimethylpyridine-N-oxide or 2-methylpyridine N-oxide; trimethylamine N-oxide, or N,N-diisopropylethylamine N-oxide;
R11 and R12 are independently selected form C1-C4 alkyl, or phenyl, preferably Formula IIIb is DMSO
optionally in the presence of an organic or inorganic base,
under conditions sufficient to form an unsaturated carbonyl product according to Formula I.

2. Method according to claim 1, further comprising the steps of
(i) forming a reaction mixture comprising:
an olefin according to Formula II: 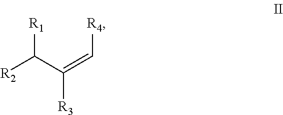
wherein R1-R4 have the meaning as described in claim 1,
a sulfoxide or sulfide according to Formula IIa or IIb: 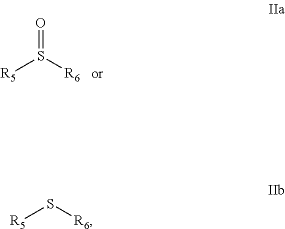
wherein R5 and R6 have the meaning as described in claim 1, more preferably the compound of formula IIa is 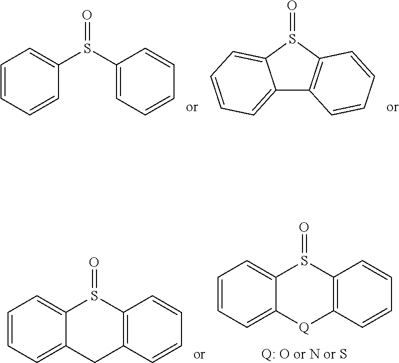
and even more preferably an optionally substituted 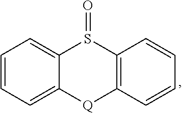
wherein Q is S (i.e. formula IIa is an optionally substituted TTO), and the optional substitution is a tetrafluoro substitution (i.e. formula IIa is 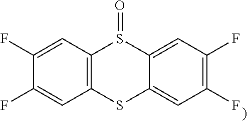 [SMILES: O=S1c2cc(F)c(F)cc2Sc2cc(F)c(F)cc21]
and the compound of formula IIb is 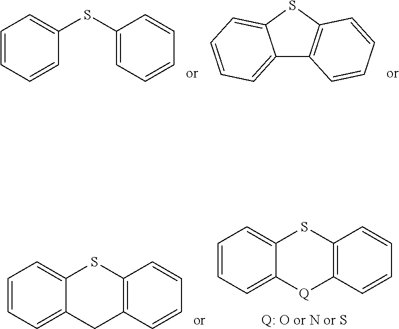
and even more preferably an optionally substituted 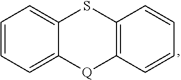
wherein Q is S (i.e. formula IIb is an optionally substituted TT), and the optional substitution is a tetrafluoro substitution (i.e. formula IIb is 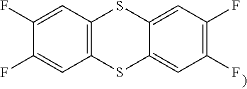 [SMILES: Fc1cc2c(cc1F)Sc1cc(F)c(F)cc1S2]
an activating agent or oxidizing agent, preferably carboxylic acid anhydride in the presence of an acid or 1-Chloromethyl-4-fluoro-1,4-diazoniabicyclo[2.2.2]octane bis(tetrafluoroborate, trifluoromethane sulfonic anhydride, or trifluoromethane sulfonic acid or methane sulfonic acid, optionally with trifluoroacetic anhydride or acetic anhydride, sodium tetrafluoroborate, tetra-n-butylammonium hexafluorophosphate, alkyl or aryl sulfonic acids, or combinations thereof, which might be replaced with an activating or oxidizing treatment (e.g. electrochemical oxidation), said agents and/or treatment provides X— which is preferably TfO⁻, MsO⁻, TsO⁻, TFA⁻, BF₄⁻, PF₆⁻, ClO₄⁻,
(ii) maintaining the reaction mixture under conditions sufficient to form an alkenyl sulfonium salt of Formula III-1, 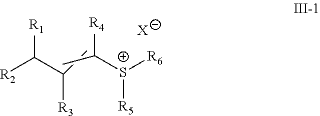
or a synthetic equivalent product including an allyl-sulfonium of Formula III-2 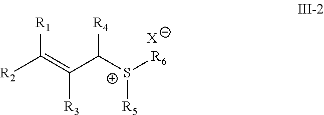
or a bis-adduct of Formula III-3 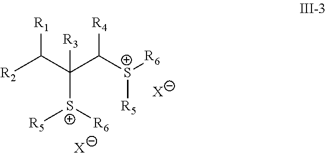
or a dicationic adduct of Formula III-4, if Formula IIa or IIb represent an optionally substituted TTO or TT 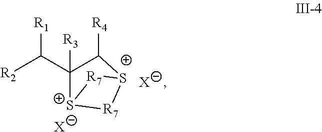
or a mixture thereof,
wherein R7 and X— have the meaning as described in claim 1.

3. Method according to claim 1, wherein
R1 is H; C1-C14 alkyl; C2-C14 alkenyl; C2-C14 alkynyl; —O—C1-C14 alkyl; —O—C2-C14 alkenyl; —O—C2-C14 alkynyl; C3-C10 cycloalkyl; 4-10-membered heterocycloalkyl containing one or more heteroatoms independently selected from O and S; C6-C10 aryl, preferably phenyl; or 5-10-membered heteroaryl containing 1-3 heteroatom independently selected from N, O and S;
wherein each alkyl and alkenyl can be straight chained or branched, and each alkyl, alkenyl, alkynyl and aryl can be optionally substituted with 1-3 substituents independently selected from halogen, —CN, —OH, —OTs, —OMs, —O—C(O)R21, ═O, —COOH, —C(O)—OR22, 1,3-dioxoisoindolin-2-yl, —NH—C(O)—C1-C6 alkyl, —N(C1-C6 alkyl)-C(O)—C1-C6 alkyl, —NHR23, —N(C1-C6 alkyl)R23, —C(O)—NH—C1-C6 alkyl, —C(O)—N(C1-C6 alkyl)₂, C3-C7 cycloalkyl, 5-7-membered heterocycloalkyl containing one or more groups independently selected from —NR23-, —O— or —S—, C6-C10 aryl, 5-7-membered heteroaryl containing 1-3 heteroatom independently selected from N, O and S, or —O—C1-C6 alkyl; or
R1 and R2 together with the carbon atom to which they are attached may form a saturated or partially unsaturated 5-6 membered cycloalkyl; or
R1 and R4 together with the carbon atoms to which they are attached may form a partially unsaturated 5-8-membered cycloalkyl; or R1 and R4 may form a chain having 2-3 chain atoms and comprises CH2 units and/or 1-2 heteroatom selected from N, O and S;
R2 is H, or together with R1, R2 may form cycloalkyl as described above;
R3 is H, C1-C4 alkyl, phenyl, 4-methyltetrahydro-2H-pyran-2-yl;
with the proviso that R1 is other than H when R2 and R3 are both H
R4 is H, C1-C4 alkyl, 4-methyltetrahydro-2H-pyran-2-yl; or together with R1, R4 may form cycloalkyl or chain as described above;
R21 is H; C1-C4 alkyl; or phenyl;
R22 is C1-C4 alkyl, C2-C4 alkenyl or C2-C4 alkynyl
R23 is N-protecting group.

4. Methods according to claim 1, wherein R5 and R6 are C6-C10 aryl, which can be substituted with one or more substituents independently selected from halogen, C1-C2 alkyl, or a —(CH2)ₙ-heteroatom-(CH2)ₘH chain where n and m is independently 0 or 1 and n+m is 0 or 1, and the heteroatom is preferably selected from N, O and S, more preferably S or oxidized heteroatom (e.g. SO, SO2, more preferably SO2); preferably R5 and R6 are both phenyl, more preferably having at least one substituent in ortho position and said substituents of the phenyls (R5 and R6) may form a direct bond between the phenyls or a chain comprising CH2 units and/or a heteroatom (preferably selected from N, O and S, more preferably S) or oxidized heteroatom (e.g. SO, SO2, more preferably SO2), more preferably the compound of formula IIa is 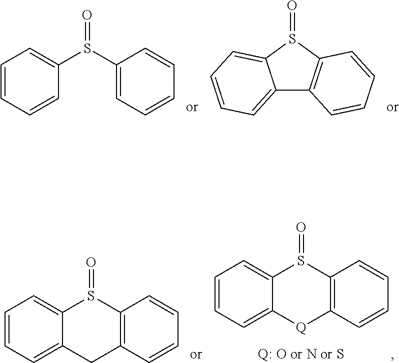
and even more preferably 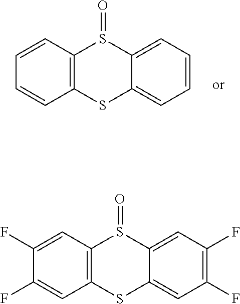 [SMILES: O=S1c2ccccc2Sc2ccccc21 ; "or" ; O=S1c2cc(F)c(F)cc2Sc2cc(F)c(F)cc21]
and the compound of formula IIb is 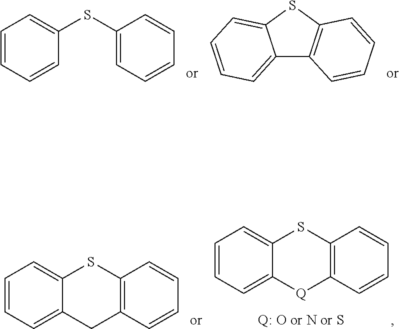
and even more preferably

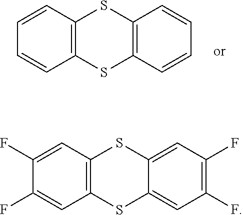 [SMILES: c1ccc2c(c1)Sc1ccccc1S2 ; "or" ; Fc1cc2c(cc1F)Sc1cc(F)c(F)cc1S2]

5. Methods according to claim 1, wherein the nucleophilic oxidizing agent is selected from 4-Methylmorpholine N-oxide, pyridine N-oxide, 2-methylpyridine N-oxide, 2,6-dimethyl-pyridine-N-oxide, trimethylamine N-oxide, N,N-diisopropylethylamine N-oxide or DMSO, preferably 4-Methylmorpholine N-oxide or DMSO.

6. Methods according to claim 1, wherein R1 is H; C1-C14 alkyl; C2-C6 alkenyl; C2-C6 alkynyl; —O—C1-C6 alkyl; or phenyl; wherein each alkyl, alkenyl and alkynyl can be straight chain or branched, and each alkyl, alkenyl, alkynyl and aryl can be substituted with 1-3 substituents independently selected from halogen, —CN, —OH, —OTs, —OMs, —O—C(O)R21, ═O, —OC1-C4 alkyl, —COOH, —C(O)—OR22, 1,3-dioxoisoindolin-2-yl or phenyl; or
R1 and R2 together with the carbon atom to which they are attached may form a saturated or partially unsaturated 5-6 membered cycloalkyl; or
R1 and R4 together with the carbon atoms to which they are attached may form a partially unsaturated 5-8 membered cycloalkyl;
R2 is H, or together with R1, R2 may form cycloalkyl as described above;
R3 is H, C1-C4 alkyl, phenyl, 4-methyltetrahydro-2H-pyran-2-yl;
with the proviso that R1 is not H when R2 and R3 are both H
R4 is H, C1-C4 alkyl, 4-methyltetrahydro-2H-pyran-2-yl; or together with R1, R4 may form cycloalkyl as described above;
R21 is H; C1-C4 alkyl; or phenyl;
R22 is C1-C4 alkyl, or C1-C4 alkynyl.

7. Methods according to claim 1, wherein R2 and R3 are H; or alternatively R3 and R4 are H; or alternatively R2, R3 and R4 are H.

8. Methods according to claim 1, wherein RI is C1-C14 alkyl; which is optionally substituted, preferably with 1-3 substituents independently selected from halogen, —CN, —OH, —OTs, —OMs, —O—C(O)R21, ═O, —OC1-C4 alkyl, —COOH, —C(O)—OR22, 1,3-dioxoisoindolin-2-yl or phenyl.

9. Methods according to claim 1, wherein the compound of Formula IIa or Formula IIb is 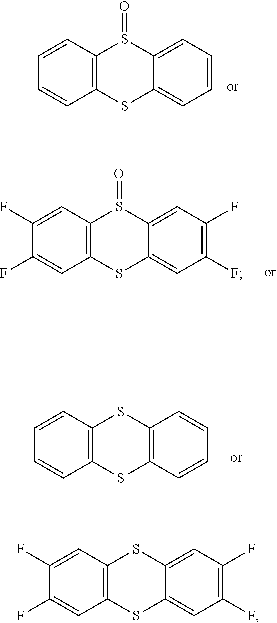 [SMILES: O=S1c2ccccc2Sc2ccccc21 ; O=S1c2cc(F)c(F)cc2Sc2cc(F)c(F)cc21 ; "or" ; c1ccc2c(c1)Sc1ccccc1S2 ; "or" ; Fc1cc2c(cc1F)Sc1cc(F)c(F)cc1S2]
respectively.

10. Methods according to claim 1, wherein the α,β-unsaturated carbonyls according to Formula I is selected from:
(Z)-pent-2-enal; (Z)-octadec-2-enal; 2-phenylacrylaldehyde; 2-cyclohexylideneacetaldehyde; (Z)-3-(3-phenylpropoxy)acrylaldehyde; (Z)-7-oxohept-5-en-1-yl benzoate; (Z)-6-oxohex-4-enenitrile; (Z)-6-(1,3-dioxoisoindolin-2-yl)hex-2-enal; (Z)-11-oxoundec-9-enoic acid; (Z)-10-hydroxydec-2-enal; (Z)-dec-2-enedial; (Z)-octa-2,7-dienal; prop-2-yn-1-yl (Z)-11-oxoundec-9-enoate; cinnamaldehyde; (Z)-6-chlorohex-2-enal; (Z)-7-bromohept-2-enal; (Z)-10-oxodec-8-en-1-yl 4-methylbenzenesulfonate; cyclopent-2-en-1-one; cyclohex-2-en-1-one; cyclohept-2-en-1-one; cyclooct-2-en-1-one; (E)-hex-3-en-2-one; (E)-hex-4-en-3-one; 2-methyl-1-((2R,4S)-4-methyltetrahydro-2H-pyran-2-yl)prop-2-en-1-one; (E)-2-methyl-3-((2R,4S)-4-methyltetrahydro-2H-pyran-2-yl) acrylaldehyde; methyl (E)-10-oxodec-8-enoate; methyl (Z)-11-oxoundec-9-enoate; (E)-9-oxonon-7-en-1-yl acetate; (Z)-11-oxoundec-9-en-1-yl acetate; (Z)-oct-2-enal; (Z)-11-hydroxyundec-2-enal.

11. Methods according to claim 1, wherein the reaction is performed in non-nucleophilic or poorly-nucleophilic solvents or solvent mixtures, preferably selected from ethers (e.g., glymes, tetrahydrofuran, methyltetrahydrofuran, diethyl ether, methyl tert-butyl ether, diisopropyl ether, dibutyl ether or 1,4-dioxane), aliphatic hydrocarbons (e.g. hexane, heptane, petroleum ether, octane, or cyclohexane), aromatic hydrocarbons (e.g. benzene, toluene, xylene or mesitylene), halocarbons (e.g. dichloromethane, chloroform, dichloroethane), carbonyls (e.g. acetone) and additional polar aprotic solvents (e.g. acetonitrile, dimethylformamide, dimethyl sulfoxide), or a mixture thereof; and/or
wherein the reaction is performed at a temperature of −40 °C to 80 °C, preferably at a temperature within the range of −20 to 50 °C; and/or
wherein the reaction is performed in the presence of an organic or inorganic base, preferably selected from N,N-diisopropylethylamine, sodium bicarbonate, sodium carbonate, potassium carbonate.

12. Methods according to claim 1, further comprising the step of transforming the compound of Formula I into conjugated diene pheromones, kairomones, and related compounds according to Formula V. 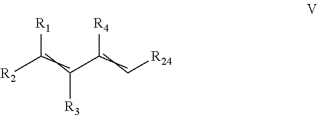
wherein R24 is H; C1-C20 alkyl, preferably C1-C14 alkyl; C2-C20 alkenyl, preferably C2-C14 alkenyl; C2-C20 alkynyl, preferably C2-C14 alkynyl; —O—C1-C20 alkyl, preferably —O—C1-C14 alkyl; or C6-C14 aryl, preferably phenyl; 5-10-membered heteroaryl containing 1-3 heteroatom independently selected from N, O and S; wherein each alkyl and alkenyl can be straight chain or branched, and each alkyl, alkenyl and aryl can be substituted with 1-3 substituents independently selected from halogen, —CN, —OH, —OTs, OMs, —O—C(O)R21, ═O, —COOH, —C(O)—OR22, 1,3-dioxoisoindolin-2-yl, —NH—CO—(C1-C6 alkyl), —N(C1-C6 alkyl)-CO—(C1-C6 alkyl), —NHR23, —N(C1-C6 alkyl)R23, —CO—NH—(C1-C6 alkyl), —CO—N(C1-C6 alkyl)₂, C6-C10 aryl, 5-10-membered heteroaryl containing 1-3 heteroatom independently selected from N, O and S, or —O—C1-C6 alkyl,
wherein the methods include:
(i) forming a reaction mixture comprising:
an unsaturated aldehyde according to Formula I,
a phosphonium ylide according to Formula IVa: 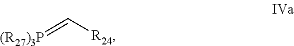
wherein R24 has the meaning as described above, and
R27 is aryl, preferably phenyl;
(ii) maintaining the reaction mixture under conditions sufficient to form a conjugated diene product according to Formula V.
wherein compound of Formula IVa is preferably generated in situ from an appropriate phosphonium salt in the presence of abase.
